SEQUENCE LISTING (102 sequences)

SEQ ID NO: 1 - protein, 146 aa, Homo sapiens
  APRIL
AVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 2 - protein, 250 aa, Homo sapiens [sequence omitted]

SEQ ID NO: 3 - protein, 146 aa, Homo sapiens; identical to SEQ ID NO: 1
  nc-APRIL

SEQ ID NO: 4 - protein, 145 aa, Artificial Sequence
  nc-APRIL
AVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVK

SEQ ID NO: 5 - protein, 144 aa, Artificial Sequence
  nc-APRIL
AVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFV

SEQ ID NO: 6 - protein, 143 aa, Artificial Sequence
  nc-APRIL
AVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGF

SEQ ID NO: 7 - protein, 122 aa, Mus musculus
  VH 104
EFQLQQSGPELVKPGASVKISCKASGYSFTDYNMSWVKQSNGKSLEWIGIISPYYGTTTYNQNFKGKATLTVDQSSSTAYMHLNSLTSEDSAVYYCARNYGGNLDYYSMDYWGQGTSVTVSS

SEQ ID NO: 8 - protein, 8 aa, Mus musculus
  CDR1 VH 104
GYSFTDYN

SEQ ID NO: 9 - protein, 8 aa, Mus musculus
  CDR2 VH 104
ISPYYGTT

SEQ ID NO: 10 - protein, 15 aa, Mus musculus
  CDR3 VH 104
ARNYGGNLDYYSMDY

SEQ ID NO: 11 - protein, 107 aa, Mus musculus
  VL 104
SIVMTQTPNFLLVSAGDRVTMTCKASQTVSNDVGWYQQKPGQSPKLLIYYASNRYTGVPDRFTGSGYGTDFTFTITTVQAEDLAVYFCQQDSSFPLTFGAGTKLELK

SEQ ID NO: 12 - protein, 6 aa, Mus musculus
  CDR1 VL 104
QTVSND

SEQ ID NO: 13 - protein, 9 aa, Mus musculus
  CDR3 VL 104
QQDSSFPLT

SEQ ID NO: 14 - protein, 113 aa, Mus musculus
  VH 108
QVQLKESGPGLVAPSQSLSITCTVSGFPLISYGVEWVRQPPGKGLEWLGIIWDDGVTNYNSALMSRLRISKDNSKRQVFLKMNSLQPDDTAIYYCARRGFAYWGQGTLVTVSA

SEQ ID NO: 15 - protein, 8 aa, Mus musculus
  CDR1 VH 108
GFPLISYG

SEQ ID NO: 16 - protein, 7 aa, Mus musculus
  CDR2 VH 108
IWDDGVT

SEQ ID NO: 17 - protein, 7 aa, Mus musculus
  CDR3 VH 108
ARRGFAY

SEQ ID NO: 18 - protein, 107 aa, Mus musculus
  VL 108
  VARIANT 4: any amino acid
DIVXTQSPSSLSASLGERVSLTCRASQEISGFLSWLQQKPDGTIKRLIYAASSLDSGVPKRFSGSRSGSDYSLTISSLESEDFADYYCLQYSTHPYTFGGGTKLEIK

SEQ ID NO: 19 - protein, 6 aa, Mus musculus
  CDR1 VL 108
QEISGF

SEQ ID NO: 20 - protein, 9 aa, Mus musculus
  CDR3 VL 108
LQYSTHPYT

SEQ ID NO: 21 - protein, 121 aa, Mus musculus
  VH 110
QVTLKESGPGILQPSQTLSLTCSFSGFSLSTFGMGVGWIRQPSGKGLEWLTHIWWDDDKYYNPALKSRLTISKDTSKNQVFLKIANVDTADTATYYCIHILDNSGYVGAYWGQGTLVTVSA

SEQ ID NO: 22 - protein, 9 aa, Mus musculus
  CDR1 VH 110
GFSLSTFGM

SEQ ID NO: 23 - protein, 7 aa, Mus musculus
  CDR2 VH 110
IWWDDDK

SEQ ID NO: 24 - protein, 13 aa, Mus musculus
  CDR3 VH 110
IHILDNSGYVGAY

SEQ ID NO: 25 - protein, 109 aa, Mus musculus
  VL 110
QAVVTQESALTTSPGETVTLTCRSSSGAVTISNFANWVQEKPDHLFTGLIAGTNNRGPGVPARFSGSLIGDRAALTITGAQTEDEAIYFCSLWYRDHWVFGGGTKLTVL

SEQ ID NO: 26 - protein, 9 aa, Mus musculus
  CDR1 VL 110
SGAVTISNF

SEQ ID NO: 27 - protein, 9 aa, Mus musculus
  CDR3 VL 110
SLWYRDHWV

SEQ ID NO: 28 - protein, 121 aa, Mus musculus
  VH 115
QVTLKESGPGILQPSQTLSLTCSFSGFSLSTFGLGVGWIRQPSGKGLEWLAHIWWDDDNYYTPALKSRLTVSKDTSKNQVFLKIANVDTADTATYYCSRAHYYDGIHFDFWGQGTTLTVSS

SEQ ID NO: 29 - protein, 10 aa, Mus musculus
  CDR1 VH 115
GFSLSTFGLG

SEQ ID NO: 30 - protein, 7 aa, Mus musculus
  CDR2 VH 115
IWWDDDN

SEQ ID NO: 31 - protein, 13 aa, Mus musculus
  CDR3 VH 115
SRAHYYDGIHFDF

SEQ ID NO: 32 - protein, 109 aa, Mus musculus
  VL 115
QTVVTQESALTTSPGETVTLTCRSSTGTVTTSNFANWVQEKPDHLFTGLISGTSNRGPGVPARFSGSLIGDKAALTITGAQTEDEAIYFCALWYSNHWVFGGGTKLTVL

SEQ ID NO: 33 - protein, 9 aa, Mus musculus
  CDR1 VL 115
TGTVTTSNF

SEQ ID NO: 34 - protein, 9 aa, Mus musculus
  CDR3 VL 115
ALWYSNHWV

SEQ ID NO: 35 - protein, 117 aa, Mus musculus
  VH 2C8
  VARIANT 3: any amino acid
EVXLEESGPELVKPGASVKISCKASGYSFTGYYMNWVKQSPEKSLEWIGEINPGTDGITFNQKFKAKATLTVDKSSSTAYMQLKSLKSEDSAVYYCATTDYYGMDYWGQGTSVTVSS

SEQ ID NO: 36 - protein, 8 aa, Mus musculus
  CDR1 VH 2C8
GYSFTGYY

SEQ ID NO: 37 - protein, 8 aa, Mus musculus
  CDR2 VH 2C8
INPGTDGI

SEQ ID NO: 38 - protein, 10 aa, Mus musculus
  CDR3 VH 2C8
ATTDYYGMDY

SEQ ID NO: 39 - protein, 112 aa, Mus musculus
  VL 2C8
  VARIANT 4: any amino acid
DIVXTQSPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKLEIK

SEQ ID NO: 40 - protein, 11 aa, Mus musculus
  CDR1 VL 2C8
QSLLDSDGKTY

SEQ ID NO: 41 - protein, 9 aa, Mus musculus
  CDR3 VL 2C8
WQGTHFPYT

SEQ ID NO: 42 - protein, 115 aa, Mus musculus
  VH Aprily 1
QIQLVQSGPELKKPGETVRISCKASGYNFKTSGMSWVKQAPGKGLKWMGWIDTYAGVPTYTDDFKGRFVFSLETSATTAFLQINNLKNEDTGTYFCAREPGTDNWGQGTSLTVSS

SEQ ID NO: 43 - protein, 8 aa, Mus musculus
  CDR1 VH Aprily 1
GYNFKTSG

SEQ ID NO: 44 - protein, 8 aa, Mus musculus
  CDR2 VH Aprily 1
IDTYAGVP

SEQ ID NO: 45 - protein, 8 aa, Mus musculus
  CDR3 VH Aprily 1
AREPGTDN

SEQ ID NO: 46 - protein, 111 aa, Mus musculus
  VL APRILY 1
DIVLTQSPASLAVSLGQRATISCRASESVDNYGISFMNWFQQKPGQPPKLLIYGASNQGSGVPARFSGSGSGTDFSLNIHPMEEDDPAMYFCQQTKEVPFTFGGGTKLEIK

SEQ ID NO: 47 - protein, 10 aa, Mus musculus
  CDR1 VL APRILY 1
ESVDNYGISF

SEQ ID NO: 48 - protein, 9 aa, Mus musculus
  CDR3 VL APRILY 1
QQTKEVPFT

SEQ ID NO: 49 - protein, 115 aa, Mus musculus
  VH APRILY 2
QIQLVQSGPELKKPGETVRISCKASGYTFKTSGMSWVKQAPGKGLKWMGWIDAYAGIPTYTDDFKGRFAFSLDTSATTAFLQINNLKNEDTGTYFCAREPGTDNWGQGTSLTVSS

SEQ ID NO: 50 - protein, 8 aa, Mus musculus
  CDR1 VH Aprily 2
GYTFKTSG

SEQ ID NO: 51 - protein, 8 aa, Mus musculus
  CDR2 VH Aprily 2
IDAYAGIP

SEQ ID NO: 52 - protein, 8 aa, Mus musculus
  CDR3 VH Aprily 2
AREPGTDN

SEQ ID NO: 53 - protein, 112 aa, Mus musculus
  VL Aprily 2
DVVMTQTPLTLSVTIGQPASISCKSSQSLLDSDGKTYLNWLLQRPGQSPKRLIYLVSKLDSGVPDRFIGSGSGTDFTLKISRVEAEDLGFYYCWQGAHFPQTFGGGTKVEIK

SEQ ID NO: 54 - protein, 11 aa, Mus musculus
  CDR1 VL Aprily 2
QSLLDSDGKTY

SEQ ID NO: 55 - protein, 9 aa, Mus musculus
  CDR3 VL Aprily 2
WQGAHFPQT

SEQ ID NO: 56 - protein, 114 aa, Mus musculus
  VH Aprily 5
EVQLEESGPELKKPGETVKISCKASGYTFTDYSVHWVRQTPGKGLKWMGWINTETGDPSYADDFKGRFAFSLETSASTAYLQIMNLKNEDTATYFCAIHAMDYWGQGTSVTVSS

SEQ ID NO: 57 - protein, 8 aa, Mus musculus
  CDR1 VH Aprily 5
GYTFTDYS

SEQ ID NO: 58 - protein, 8 aa, Mus musculus
  CDR2 VH Aprily 5
INTETGDP

SEQ ID NO: 59 - protein, 7 aa, Mus musculus
  CDR3 VH Aprily 5
AIHAMDY

SEQ ID NO: 60 - protein, 107 aa, Mus musculus
  VL Aprily 5
  VARIANT 4: any amino acid
DIVXTQSPSSLSASLGDTITITCHASQNINVWLTWYQQKPGNIPKLLIYKASNLHTGVPSRFSGSGSGTGFTLTISSLQPEDIATYYCQQGHAYPLTFGGGTKLEIR

SEQ ID NO: 61 - protein, 6 aa, Mus musculus
  CDR1 VL Aprily 5
QNINVW

SEQ ID NO: 62 - protein, 9 aa, Mus musculus
  CDR3 VL Aprily 5
QQGHAYPLT

SEQ ID NO: 63 - protein, 6 aa, Homo sapiens
  Epitope on APRIL
HLVPNI

SEQ ID NO: 64 - protein, 6 aa, Homo sapiens
  Epitope on APRIL
PALRRG

SEQ ID NO: 65 - DNA, 366 bp, Mus musculus
  VH 104
gagttccagctgcagcagtctggacctgagctggtgaagcctggcgcctcagtgaagatttcctgcaaggcttctggttactctttcactgactacaatatgagctgggtgaagcagagcaatggaaagagccttgaatggattggaataattagtccttactatggtactactacctacaatcagaacttcaagggcaaggccacattgactgtagaccaatcttccagcacagcctacatgcacctcaacagcctgacatctgaggactctgcagtctattactgtgcaagaaactacggtggtaacttggattattatagtatggactactggggtcaaggaacctcagtcaccgtctcctca

SEQ ID NO: 66 - DNA, 321 bp, Mus musculus
  VL 104
agtattgtgatgacccagactcccaacttcctgcttgtttcagcaggagacagggttaccatgacctgcaaggccagtcagactgtgagtaatgatgtaggttggtaccaacagaagcctgggcagtctcctaaactgctgatatattatgcatccaatcgctacactggagtccctgatcgcttcactggcagtggatatgggacggatttcactttcaccatcaccactgtgcaggcggaagacctggcagtttatttctgtcagcaggattctagttttccgctcacgttcggtgctgggaccaagctggaaataaaa

SEQ ID NO: 67 - DNA, 339 bp, Mus musculus
  VH 108
caggtgcagctgaaggagtcaggacctggcctggtggcgccctcacagagcctgtccatcacttgcactgtctctgggtttccattaatcagctatggtgtagagtgggttcgccagcctccaggaaagggtctggagtggctgggaatcatatgggatgatggagtcacaaattataattcagctctcatgtccagactgcggatcagcaaagacaactccaagcgccaagtcttcttaaaaatgaatagtctccaacctgatgacacagccatatactattgtgccagaagaggttttgcttactggggccaagggactctggtcactgtctctgca

SEQ ID NO: 68 - DNA, 321 bp, Mus musculus
  VL 108
  variation 10: /replace=“any nucleotide”
gatattgtgntgacccagtctccatcctccttatctgcctctctgggagaaagagtcagtctcacttgtcgggcaagtcaggaaattagtggtttcttaagctggcttcagcagaaaccagatggaactatcaaacgcctgatctacgccgcatccagtttagattctggtgtcccaaaaaggttcagtggcagtaggtctgggtcagattattctctcaccatcagcagccttgagtctgaagattttgcagactattactgtctacaatattctactcatccgtacacgttcggaggggggaccaagctggaaataaaa

SEQ ID NO: 69 - DNA, 363 bp, Mus musculus
  VH 110
caggttactctgaaagagtctggccctgggatattgcagccctcccagaccctcagtctgacttgttctttctctgggttttcactgagcacttttggtatgggtgtaggctggattcgtcagccttcagggaagggtctggagtggctgacacacatttggtgggatgatgataagtactataatccagccctgaagagtcggctcacaatctccaaggatacctccaaaaaccaggtattcctcaagatcgccaatgtggacactgcagatactgccacatactactgtattcacatcctagacaactcaggttacgtgggcgcttactggggccaagggactctggtcactgtctctgca

SEQ ID NO: 70 - DNA, 327 bp, Mus musculus
  VL 110
caggctgttgtgactcaggaatctgcactcaccacatcacctggtgaaacagtcacactcacttgtcgctcaagttctggggctgttacaatcagtaactttgccaactgggtccaagaaaaaccagatcatttattcactggtctaatagctggtaccaacaaccgaggtccaggtgttcctgccagattctcaggctccctgattggagacagggctgccctcaccatcacaggggcacagactgaggatgaggcaatatatttctgttctctatggtacagagaccactgggtgttcggtggaggaaccaaactgactgtccta

SEQ ID NO: 71 - DNA, 363 bp, Mus musculus
  VH 115
caggttactctgaaagagtctggccctgggatattgcagccctcccagaccctcagtctgacttgttctttctctgggttttcactcagcacttttggtttgggtgtaggctggattcgtcagccttcagggaagggtctggagtggctggcacacatttggtgggatgatgataactactataccccggccctgaagagtcggctcacagtctccaaggatacctccaaaaaccaggttttcctcaagatcgccaatgtggacactgcagatactgccacatactactgttctcgcgcccattactatgatggtatccactttgacttctggggccaaggcaccactctcacagtctcctca

SEQ ID NO: 72 - DNA, 327 bp, Mus musculus
  VL 115
cagactgttgtgactcaggaatctgcactcaccacatcccctggtgaaacagtcacactcacttgtcgctcaagtactgggactgttacaactagtaactttgccaactgggtccaagaaaaaccagatcatttattcactggtctaataagtggtaccagcaaccgaggtccaggtgttcctgccagattctcaggctccctgattggagacaaggctgccctcaccatcacaggggcacagactgaggatgaggcaatatatttctgtgctctatggtacagcaatcactgggtgttcggtggaggaaccaaactgactgtccta

SEQ ID NO: 73 - DNA, 351 bp, Mus musculus
  VH 2C8
  variation 6: /replace=“any nucleotide”
  variation 7: /replace=“any nucleotide”
gaggtnnagctggaggagtctggacctgaactggtgaagcctggggcttcagtgaagatatcctgcaaggcttctggttactcattcactggctattacatgaactgggtgaagcaaagtcctgaaaagagccttgagtggattggagagattaatcctggcactgatggtattacctttaaccagaagttcaaggccaaggccacattgactgtagacaaatcctccagcacagcctacatgcagctcaaaagcctgaaatctgaggactctgcagtctattactgtgcgactacggattactatggcatggactactggggtcaaggaacctcagtcaccgtctcctca

SEQ ID NO: 74 - DNA, 336 bp, Mus musculus
  VL 2C8
gatattgtgmtgacccagtctccactcactttgtcggttaccattggacaaccagcctccatctcttgcaagtcaagtcagagcctcttagatagtgatggaaagacatatttgaattggttattacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacccattttccgtacacgttcggaggggggaccaagctggaaataaaa

SEQ ID NO: 75 - DNA, 345 bp, Mus musculus
  VH Aprily 1
cagatccagttggtacagtctggacctgaactgaagaagcctggagagacagtcaggatctcctgcaaggcctctggctataacttcaaaacctctggaatgagctgggtgaaacaggcaccaggaaagggtttaaagtggatgggctggatagacacctacgctggagtgccaacatatactgatgacttcaagggacggtttgtcttctctttggaaacctctgccaccactgcctttttgcagatcaacaacctcaaaaatgaggacacgggaacatatttctgtgcaagagaacctgggacagacaactggggccaaggcacatctctcactgtctcctca

SEQ ID NO: 76 - DNA, 333 bp, Mus musculus
  VL Aprily 1
gacattgtgctgacccaatctccagcttctttggctgtgtctctagggcagagggccaccatctcctgcagagccagcgaaagtgttgataattatggcattagttttatgaactggttccaacagaaaccaggacagccacccaaactcctcatctatggtgcatccaaccaaggatccggggtccctgccaggtttagtggcagtggatctgggacagacttcagcctcaacatccatcctatggaggaggatgatcctgcaatgtatttctgtcagcaaactaaggaggttccgttcacgttcggaggggggaccaagctggaaataaaa

SEQ ID NO: 77 - DNA, 345 bp, Mus musculus
  VH Aprily 2
cagatccagttggtacagtctggacctgagctgaagaagcctggagagacagtcaggatctcctgcaaggcctctggctataccttcaaaacctctggaatgagctgggtgaaacaggcaccaggaaagggtttaaagtggatgggctggatagacgcctacgctggaataccaacatatactgatgacttcaagggacggtttgccttctctttggacacctctgccaccactgcctttttgcagatcaacaacctcaaaaatgaggacacgggaacatatttctgtgcaagagaacctgggacagacaactggggccaaggcacatctctcactgtctcctca

SEQ ID NO: 78 - DNA, 336 bp, Mus musculus
  VL Aprily 2
gatgtcgtgatgacccagaccccactcactttgtcggttaccattggacaaccagcctccatttcttgtaagtcaagtcagagcctcttagatagtgatggaaagacatatttgaattggttgttacagaggccaggccagtctccaaagcgcctaatctatctggtgtctaaactggactctggagtccctgacaggttcattggcagtggatcagggacagatttcacactgaaaatcagcagagtggaggctgaggatttgggattttattattgctggcaaggtgcacattttcctcagacgttcggtggaggcaccaaggtggaaatcaaa

SEQ ID NO: 79 - DNA, 342 bp, Mus musculus
  VH Aprily 5
  variation 6: /replace=“any nucleotide”
gaggtncagctggaggagtctggacctgagctgaaaaagcctggagagacagtcaagatctcctgcaaggcttctggttataccttcacagactattcagtgcactgggtgaggcaaactccaggcaagggtttaaagtggatgggctggataaacactgagactggtgacccatcatatgcagatgacttcaagggacggtttgccttctctttggaaacctctgccagcactgcctatttgcagatcatgaacctcaaaaatgaggacacggctacatatttctgtgctatccatgctatggactactggggtcaaggaacctcagtcaccgtctcctca

SEQ ID NO: 80 - DNA, 321 bp, Mus musculus
  VL Aprily 5
gatattgtgmtgacccagtctccatccagtctgtctgcatcccttggagacacaattaccatcacttgccatgccagtcagaacattaatgtttggttaacctggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcatgcttatcctctgacgttcggtggaggcaccaagctggaaatcaga

SEQ ID NO: 81 - DNA, 411 bp, Artificial Sequence
  ps1307
cactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtga

SEQ ID NO: 82 - DNA, 408 bp, Artificial Sequence
  ps4185
cactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaatga

SEQ ID NO: 83 - DNA, 405 bp, Artificial Sequence
  ps4186
cactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgtga

SEQ ID NO: 84 - DNA, 402 bp, Artificial Sequence
  ps4187
cactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggttttga

SEQ ID NO: 85 - DNA, 399 bp, Artificial Sequence
  ps4188
cactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtga

SEQ ID NO: 86 - DNA, 1230 bp, Artificial Sequence
  ps1307 (full Fc-APRIL)
atggctatcatctacctcatcctcctgttcaccgctgtgcggggcctcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaagatctccgcagccgcagccgaaaccgcagccgaaaccggaaccggaaggatctctggaggtgctgttccaggggcccggatccctgcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtga

SEQ ID NO: 87 - DNA, 1227 bp, Artificial Sequence
  ps4185 (full Fc-APRIL)
atggctatcatctacctcatcctcctgttcaccgctgtgcggggcctcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaagatctccgcagccgcagccgaaaccgcagccgaaaccggaaccggaaggatctctggaggtgctgttccaggggcccggatccctgcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaatga

SEQ ID NO: 88 - DNA, 1224 bp, Artificial Sequence
  ps4186 (full Fc-APRIL)
atggctatcatctacctcatcctcctgttcaccgctgtgcggggcctcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaagatctccgcagccgcagccgaaaccgcagccgaaaccggaaccggaaggatctctggaggtgctgttccaggggcccggatccctgcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgtga

SEQ ID NO: 89 - DNA, 1221 bp, Artificial Sequence
  ps4187 (full Fc-APRIL)
atggctatcatctacctcatcctcctgttcaccgctgtgcggggcctcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaagatctccgcagccgcagccgaaaccgcagccgaaaccggaaccggaaggatctctggaggtgctgttccaggggcccggatccctgcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggttttga

SEQ ID NO: 90 - DNA, 1218 bp, Artificial Sequence
  ps4188 (full Fc-APRIL)
atggctatcatctacctcatcctcctgttcaccgctgtgcggggcctcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaagatctccgcagccgcagccgaaaccgcagccgaaaccggaaccggaaggatctctggaggtgctgttccaggggcccggatccctgcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtga

SEQ ID NO: 91 - protein, 409 aa, Artificial Sequence [sequence omitted]

SEQ ID NO: 92 - protein, 408 aa, Artificial Sequence
  ps4185 ( (full Fc-APRIL)
MAIIYLILLFTAVRGLDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRSPQPQPKPQPKPEPEGSLEVLFQGPGSLQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVK

SEQ ID NO: 93 - protein, 407 aa, Artificial Sequence
  ps4186 (full Fc-APRIL)
MAIIYLILLFTAVRGLDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRSPQPQPKPQPKPEPEGSLEVLFQGPGSLQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFV

SEQ ID NO: 94 - protein, 406 aa, Artificial Sequence
  ps4187 (full Fc-APRIL)
MAIIYLILLFTAVRGLDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRSPQPQPKPQPKPEPEGSLEVLFQGPGSLQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGF

SEQ ID NO: 95 - protein, 405 aa, Artificial Sequence
  ps4188 (full Fc-APRIL)
MAIIYLILLFTAVRGLDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRSPQPQPKPQPKPEPEGSLEVLFQGPGSLQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLG

SEQ ID NO: 96 - protein, 7 aa, Homo sapiens
  Epitope 5 on APRIL
HLVPINA

SEQ ID NO: 97 - protein, 7 aa, Mus musculus
  Epitope 5 on APRIL
HLVPVNI

SEQ ID NO: 98 - protein, 6 aa, Mus musculus
  Epitope 1,2 on APRIL
PVLRRG

SEQ ID NO: 99 - protein, 9 aa, Artificial Sequence
  tryptic peptide in the Fc part
EEQYNSTYR

SEQ ID NO: 100 - protein, 14 aa, Artificial Sequence
  tryptic C terminus APRIL
LNLSPHGTFLGFVK

SEQ ID NO: 101 - protein, 15 aa, Artificial Sequence
  miscleaved tryptic C terminus APRIL
LNLSPHGTFLGFVKL

SEQ ID NO: 102 - protein, 394 aa, Artificial Sequence
  Sequence on Figure 23 (ps1307, Fc-APRIL, mature protein)
LDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRSPQPQPKPQPKPEPEGSLEVLFQGPGSLQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

(19) United States
(12) Patent Application Publication
(10) Pub. No.: US 2024/0101692 A1
(43) Pub. Date: Mar. 28, 2024

(54) ANTI-APRIL ANTIBODIES AND USES THEREOF

(71) Applicants: CeMM - FORSCHUNGSZENTRUM FÜR MOLEKULARE MEDIZIN GMBH, Wien (AT); MEDIZINISCHE UNIVERSITÄT WIEN, Wien (AT); UNIVERSITÉ DE LAUSANNE, Lausanne (CH)

(72) Inventors: Dimitris TSIANTOULAS, Wien (AT); Christoph BINDER, Wien (AT); Pascal SCHNEIDER, Lausanne (CH); Mahya ESLAMI, Lausanne (CH)

(21) Appl. No.: 18/267,288

(22) PCT Filed: Dec. 29, 2021
(86) PCT No.: PCT/EP2021/087779

§ 371 Date: Jul. 7, 2023

(30) Foreign Application Priority Data
Dec. 29, 2020 (EP) 20217536.0

(51) Int. Cl. C07K 16/28; A61P 3/06; G01N 33/543; G01N 33/68

(52) U.S. Cl. CPC C07K 16/2875; A61P 3/06; G01N 33/54386; G01N 33/6863; A61K 2039/505; C07K 2317/76; G01N 2800/042; G01N 2800/08; G01N 2800/52

(57) ABSTRACT

The present invention relates to an antibody, or an antigen-binding fragment thereof, specifically binding to APRIL for use in the prevention and/or treatment of hypertriglyceridemia, metabolic syndrome, non-alcoholic steatohepatitis, diabetes mellitus type 2, abdominal aortic aneurysm, atherogenic dyslipidemia, cardiovascular events (e.g., myocardial infarction and stroke) and/or atherosclerosis. The invention further relates to a polynucleotide that encodes and/or a pharmaceutical composition that comprises the antibody or an antigen-binding fragment of the invention. The invention also relates to a kit and/or method for quantifying the concentration of nc-APRIL, canonical APRIL or total APRIL in a sample. Further, the invention relates to a nephelometric assay for quantifying nc-APRIL. Further, the invention relates to a method for predicting mortality risk in subjects suffering from, and/or for determining whether a subject is susceptible to the treatment of hypertriglyceridemia, metabolic syndrome, abdominal aortic aneurysm, non-alcoholic steatohepatitis, diabetes mellitus type 2, atherogenic dyslipidemia, cardiovascular events and/or atherosclerosis.

The present invention relates to an antibody, or an antigen-binding fragment thereof, specifically binding to APRIL for use in the prevention and/or treatment of hypertriglyceridemia, metabolic syndrome, non-alcoholic steatohepatitis, diabetes mellitus type 2, abdominal aortic aneurysm, atherogenic dyslipidemia, cardiovascular events (e.g., myocardial infarction and stroke) and/or atherosclerosis.

The invention further relates to a polynucleotide that encodes and/or a pharmaceutical composition that comprises the antibody or an antigen-binding fragment of the invention. The invention also relates to a kit and/or method for quantifying the concentration of nc-APRIL, canonical APRIL or total APRIL in a sample. Further, the invention relates to a nephelometric assay for quantifying nc-APRIL. Further, the invention relates to a method for predicting mortality risk in subjects suffering from, and/or for determining whether a subject is susceptible to the treatment of hypertriglyceridemia, metabolic syndrome, abdominal aortic aneurysm, non-alcoholic steatohepatitis, diabetes mellitus type 2, atherogenic dyslipidemia, cardiovascular events and/or atherosclerosis.

Triglycerides (TGs) are the most important source of energy in the body. TGs that are derived from dietary fat (exogenous synthesis pathway) are merged with Apolipoprotein 48 in the enterocytes and are subsequently transported in the blood in the form of chylomicrons. Lipoprotein lipase (LPL), which is arrested on proteoglycans (PGs) present on the endothelial side of blood capillaries, hydrolyses TGs to produce free fatty acids. In addition to the exogenous synthesis, TGs are also produced endogenously by hepatocytes and, in combination with Apolipoprotein B100, form very low-density lipoprotein (VLDL) particles. VLDL and TGs can be hydrolyzed by LPL, leading to the formation of smaller particles such as low-density lipoproteins (LDL) that are richer in cholesterol content (Reiner, Z. Hypertriglyceridaemia and risk of coronary artery disease; Nat Rev Cardiol 14, 401-411 (2017); Toth, P. P. Triglyceride-rich lipoproteins as a causal factor for cardiovascular disease. Vasc Health Risk Manag 12, 171-183 (2016); Zechner, R., Madeo, F. & Kratky, D. Cytosolic lipolysis and lipophagy: two sides of the same coin. Nat Rev Mol Cell Biol 18, 671-684 (2017)). Hypertriglyceridemia manifests when plasma levels of triglycerides exceed 150 mg/dl. Its prevalence in Europe and North America is high, with 25% of adults having >170 mg/dl of non-fasting triglyceride levels content (Reiner, Z. Hypertriglyceridaemia and risk of coronary artery disease. Nat Rev Cardiol 14, 401-411 (2017); Toth, P. P. Triglyceride-rich lipoproteins as a causal factor for cardiovascular disease. Vasc Health Risk Manag 12, 171-183 (2016); Brahm, A. J. & Hegele, R. A. Chylomicronaemia—current diagnosis and future therapies. Nat Rev Endocrinol 11, 352-362 (2015)). Such deviation from a homeostatic balance of triglyceride metabolism can contribute to life-threatening pathologies (Reiner, Z. Hypertriglyceridaemia and risk of coronary artery disease. Nat Rev Cardiol 14, 401-411 (2017); Toth, P. P. Triglyceride-rich lipoproteins as a causal factor for cardiovascular disease. Vasc Health Risk Manag 12, 171-183 (2016)), such as heart attacks and strokes, which are the leading causes of mortality and morbidity worldwide (World Health Organization. Global status report on noncommunicable diseases 2014. xvii, 280 pages (World Health Organization, Geneva, Switzerland, 2014); WHO. World Health Organization on Cardiovascular diseases 2016. Published online at (2017)). The main underlying pathology of these catastrophic clinical manifestations is atherosclerotic cardiovascular disease (CVD), which leads to the formation of an atherosclerotic plaque in large and medium-size arteries. Atherosclerosis is initiated upon trapping of LDL in the proteoglycan-rich matrix of the subendothelial space (Hansson, G. K. & Hermansson, A. The immune system in atherosclerosis. Nat Immunol 12, 204-212 (2011); Ridker, P. M., et al. Antiinflammatory Therapy with Canakinumab for Atherosclerotic Disease. N Engl J Med (2017)), thereby triggering a chronic inflammatory and remodeling response of the artery wall that ultimately results in the formation of an atherosclerotic plaque (Libby, P., Lichtman, A. H. & Hansson, G. K. Immune effector mechanisms implicated in atherosclerosis: from mice to humans. Immunity 38, 1092-1104 (2013)). In addition, PGs also intervene with lipoprotein metabolism by facilitating the arrest of chylomicrons and VLDL at the luminal surface and thereby promoting hydrolysis of their TGs content by the endothelial cell-bound LPL. This results in the formation of small lipoproteins such as LDL that can easily penetrate the endothelium barrier and initiate atherosclerotic plaque formation.

LDL cholesterol lowering therapies significantly reduce the clinical consequences of atherosclerotic CVD, however a high risk still remains (Randomised trial of cholesterol lowering in 4444 patients with coronary heart disease: the Scandinavian Simvastatin Survival Study (4S). Lancet 344, 1383-1389 (1994)). Moreover, even in patients that are treated with a combination of statins and PCSK9 inhibitors and achieve very low LDL cholesterol levels, a particularly significant CVD risk remains (Pradhan et al, Residual Inflammatory Risk on Treatment With PCSK9 Inhibition and Statin Therapy, Circulation, 2018).

Based on epidemiological and genetic data, increased levels of triglycerides represent an independent causal risk factor for CVD (Nordestgaard, B. G. Triglyceride-Rich Lipoproteins and Atherosclerotic Cardiovascular Disease: New Insights From Epidemiology, Genetics, and Biology. Circ Res 118, 547-563 (2016)). In the clinic, treatment for lowering triglycerides includes life style changes and administration of fibrates combined with statins that can reduce TGs levels by 25-50%, depending on the baseline levels (Chapman, M. J., et al. Triglyceride-rich lipoproteins and high-density lipoprotein cholesterol in patients at high risk of cardiovascular disease: evidence and guidance for management. Eur Heart J 32, 1345-1361 (2011)).

Additional therapies are critically needed to achieve a more efficient therapeutic management of TGs levels and combat human atherosclerotic CVD. In this regard, current studies investigate the effect of inhibiting the expression of Angiopoietin-like protein 3 (ANGPTL3) and Apolipoprotein C-III (apoC-III), which both inhibit LPL activity and prevent TGs degradation (Olkkonen, V. M., Sinisalo, J. & Jauhiainen, M. New medications targeting triglyceride-rich lipoproteins: Can inhibition of ANGPTL3 or apoC-III reduce the residual cardiovascular risk? Atherosclerosis 272, 27-32 (2018)). Furthermore, a recent study has demonstrated the presence, in individuals with high triglyceride levels, of blocking auto-antibodies against the GPIHBP1 protein, which prevent the binding of LPL to GPIHBP1 and thus the translocation of LPL into the capillary lumen where it can hydrolyze TGs (Beigneux, A. P., et al. Autoantibodies against GPIHBP1 as a Cause of Hypertriglyceridemia. N Engl J Med 376, 1647-1658 (2017)).

APRIL (A Proliferation Inducing Ligand) is a cytokine produced by various cell types (such as stromal cells, monocytes and macrophages) and plays an important role in sustaining plasmablasts and antibody-secreting plasma cells, particularly IgA-producing cells in the gut (Mackay, F. & Schneider, P. Cracking the BAFF code. Nat Rev Immunol 9, 491-502 (2009); Castigli, E., et al. Impaired IgA class switching in APRIL-deficient mice. Proc Natl Acad Sci USA 101, 3903-3908 (2004)). These properties of APRIL are mediated via activating its cognate receptors TACI (transmembrane activator and CAML interactor) and BCMA (B cell maturation antigen), which are predominately present on B cells (Mackay, F. & Schneider, P. Cracking the BAFF code. Nat Rev Immunol 9, 491-502 (2009); Vincent, F. B., Morand, E. F., Schneider, P. & Mackay, F. The BAFF/APRIL system in SLE pathogenesis. Nat Rev Rheumatol 10, 365-373 (2014)). Notably, APRIL also binds to PGs (Ingold, K., et al. Identification of proteoglycans as the APRIL-specific binding partners. J Exp Med 201, 1375-1383 (2005); Huard, B., et al. APRIL secreted by neutrophils binds to heparan sulfate proteoglycans to create plasma cell niches in human mucosa. J Clin Invest 118, 2887-2895 (2008)), but the physiological role of this interaction remains largely elusive.

Accordingly, there is a need for agents with new targets for the diagnosis, prevention and/or treatment of adverse events secondary to disease-related cardiovascular process such as hypertriglyceridemia, metabolic syndrome, non-alcoholic steatohepatitis, diabetes mellitus type 2, atherogenic dyslipidemia, cardiovascular events and/or atherosclerosis. The above technical problem is solved by the embodiments as defined in the claims.

Accordingly, the invention relates to, inter alia, the following embodiments:
1. An antibody, or an antigen-binding fragment thereof, specifically binding to APRIL for use in the prevention and/or treatment of hypertriglyceridemia, metabolic syndrome, non-alcoholic steatohepatitis, diabetes mellitus type 2, atherogenic dyslipidemia, abdominal aortic aneurysm, cardiovascular events and/or atherosclerosis.
2. The antibody, or antigen-binding fragment thereof, according to embodiment 1, for use in the treatment and or prevention of hypertriglyceridemia.
3. The antibody, or antigen-binding fragment thereof, for use of embodiment 2, wherein the hypertriglyceridemia is at least one selected from the group of hypertriglyceridemia in metabolic syndrome, hypertriglyceridemia in non-alcoholic steatohepatitis-related, hypertriglyceridemia in diabetes mellitus type 2, hypertriglyceridemia in atherogenic, cardiovascular events with a history of hypertriglyceridemia and atherosclerosis with hypertriglyceridemia.
4. The antibody, or antigen-binding fragment thereof, according to embodiment 1, for use in the treatment and or prevention of abdominal aortic aneurysm.
5. The antibody, or antigen-binding fragment thereof, for use according to embodiment 1, wherein cardiovascular events comprise myocardial infarction, stroke, peripheral artery disease, angina pectoris and/or urgent hospitalization for angina leading to revascularization.
6. The antibody, or antigen-binding fragment thereof, for use according to embodiment 1 to 5, wherein the binding of the antibody or antigen-binding fragment thereof to APRIL results in an increased interaction of APRIL with proteoglycans.
7. The antibody, or antigen-binding fragment thereof, for use according to embodiment 6, wherein the proteoglycans are arterial and vascular proteoglycans.
8. The antibody, or antigen-binding fragment thereof, for use according to embodiments 6 or 7, wherein the proteoglycans are comprised in an extracellular matrix.
9. The antibody, or antigen-binding fragment thereof, according to any one of embodiments 1 to 8, wherein the binding of the antibody or antigen-binding fragment thereof to APRIL modulates the interaction of APRIL with at least one of its endogenous receptors.
10. The antibody, or antigen-binding fragment thereof, according to embodiment 9, wherein the binding of the antibody or antigen-binding fragment thereof to APRIL blocks the interaction of APRIL with the receptors TACI and BCMA.
11. An antibody, or antigen-binding fragment thereof or the antibody, or antigen-binding fragment thereof, for use according to any one of embodiments 1 to 10, wherein the antibody or antigen-binding fragment thereof
   (a) comprises a variable heavy (VH) chain comprising CDR3 as defined in SEQ ID NO:10 and a variable light (VL) chain comprising CDR3 as defined in SEQ ID NO:13;
   (b) comprises a variable heavy (VH) chain comprising CDR3 as defined in SEQ ID NO:17 and a variable light (VL) chain comprising CDR3 as defined in SEQ ID NO:20;
   (c) comprises a variable heavy (VH) chain comprising CDR3 as defined in SEQ ID NO:24 and a variable light (VL) chain comprising CDR3 as defined in SEQ ID NO:27;
   (d) comprises a variable heavy (VH) chain comprising CDR3 as defined in SEQ ID NO:31 and a variable light (VL) chain comprising CDR3 as defined in SEQ ID NO:34; or
   (e) comprises a variable heavy (VH) chain comprising CDR3 as defined in SEQ ID NO:38 and a variable light (VL) chain comprising CDR3 as defined in SEQ ID NO:41.
12. The antibody, or antigen-binding fragment thereof, according to embodiment 11 or for use according to any one of embodiments 1 to 11, wherein the antibody or antigen-binding fragment thereof
   (a) comprises a variable heavy (VH) chain comprising CDR1 as defined in SEQ ID NO:8, CDR2 as defined in SEQ ID NO:9 and CDR3 as defined in SEQ ID NO:10 and a variable light (VL) chain comprising CDR1 as defined in SEQ ID NO:12, CDR2 as defined in the sequence: YAS and CDR3 as defined in SEQ ID NO: 13;
   (b) comprises a variable heavy (VH) chain comprising CDR1 as defined in SEQ ID NO:15, CDR2 as defined in SEQ ID NO:16 and CDR3 as defined in SEQ ID NO:17 and a variable light (VL) chain comprising CDR1 as defined in SEQ ID NO:19, CDR2 as defined by the amino acid sequence: AAS and CDR3 as defined in SEQ ID NO:20;
   (c) comprises a variable heavy (VH) chain comprising CDR1 as defined in SEQ ID NO:22, CDR2 as defined in SEQ ID NO:23 and CDR3 as defined in SEQ ID NO:24 and a variable light (VL) chain comprising CDR1 as defined in SEQ ID NO:26, CDR2 as defined by the amino acid sequence: GTN and CDR3 as defined in SEQ ID NO:27;
   (d) comprises a variable heavy (VH) chain comprising CDR1 as defined in SEQ ID NO:29, CDR2 as defined in SEQ ID NO:30 and CDR3 as defined in SEQ ID NO:31 and a variable light (VL) chain comprising CDR1 as defined in SEQ ID NO:33, CDR2 as defined by the amino acid sequence: GTS and CDR3 as defined in SEQ ID NO:34; or
   e) comprises a variable heavy (VH) chain comprising CDR1 as defined in SEQ ID NO:36, CDR2 as defined in SEQ ID NO:37 and CDR3 as defined in SEQ ID NO:38 and a variable light (VL) chain comprising CDR1 as defined in SEQ ID NO:40, CDR2 as defined by the amino acid sequence: LVS and CDR3 as defined in SEQ ID NO:41.
13. The antibody, or antigen-binding fragment thereof, according to embodiment 11 or 12 or for use according to any one of embodiments 1 to 12, wherein the antibody or antigen-binding fragment thereof
   (a) comprises a variable heavy (VH) chain sequence comprising the amino acid sequence of SEQ ID NO:7 or a sequence having 90%, preferably 95% sequence identity to SEQ ID NO:7; and a variable light (VL) chain sequence comprising the amino acid sequence of SEQ ID NO:11 or a sequence having 90%, preferably 95% sequence identity to SEQ ID NO: 11;
      (b) comprises a variable heavy (VH) chain sequence comprising the amino acid sequence of SEQ ID NO:14 or a sequence having 90%, preferably 95% sequence identity to SEQ ID NO:14; and
      a variable light (VL) chain sequence comprising the amino acid sequence of SEQ ID NO:18 or a sequence having 90%, preferably 95% sequence identity to SEQ ID NO:18;
   (c) comprises a variable heavy (VH) chain sequence comprising the amino acid sequence of SEQ ID NO:21 or a sequence having 90%, preferably 95% sequence identity to SEQ ID NO:21; and
      a variable light (VL) chain sequence comprising the amino acid sequence of SEQ ID NO:25 or a sequence having 90%, preferably 95% sequence identity to SEQ ID NO:25;
   (d) comprises a variable heavy (VH) chain sequence comprising the amino acid sequence of SEQ ID NO:28 or a sequence having 90%, preferably 95% sequence identity to SEQ ID NO:28; and
      a variable light (VL) chain sequence comprising the amino acid sequence of SEQ ID NO:32 or a sequence having 90%, preferably 95% sequence identity to SEQ ID NO:32; or
   (e) comprises a variable heavy (VH) chain sequence comprising the amino acid sequence of SEQ ID NO:35 or a sequence having 90%, preferably 95% sequence identity to SEQ ID NO:35; and
      a variable light (VL) chain sequence comprising the amino acid sequence of SEQ ID NO:39 or a sequence having 90%, preferably 95% sequence identity to SEQ ID NO:39.
14. The antibody, or antigen-binding fragment thereof, according to any one of embodiments 11 to 13 or for use according to any one of embodiments 1 to 13, wherein the antibody is an IgM, IgG1, IgG2a or IgG2b, IgG3, IgG4, IgA or IgE antibody.
15. The antibody, or antigen-binding fragment thereof, according to any one of embodiments 11 to 14 or for use according to any one of embodiments 1 to 14, wherein the antigen-binding fragment is a Fab fragment, an F(ab′) fragment, an Fv fragment or an scFv fragment.
16. A polynucleotide that encodes the antibody, or an antigen-binding fragment thereof, according to any one of embodiments 11 to 15.
17. The polynucleotide according to claim 16, wherein the polynucleotide encodes the antibody, or an antigen-binding fragment thereof, according to embodiment 13.
18. A pharmaceutical composition comprising the antibody, or antigen-binding fragment thereof, according to any one of embodiments 11 to 15 and a pharmaceutically acceptable carrier.
19. The pharmaceutical composition according to embodiment 18, comprising a further therapeutic agent.
20. The pharmaceutical composition according to embodiment 19, wherein the further therapeutic agent is selected from a group consisting of: fibrates, statins, agents that inhibit the expression of Angiopoietin-like protein 3 (ANGPTL3) or Apolipoprotein C-III (apoC-III), and agents that prevent the binding of auto-antibodies to GPIHBP1.
21. A method for quantifying the concentration of non-canonical APRIL (nc-APRIL) in a sample, the method comprising the steps of:
   a) contacting the sample comprising nc-APRIL with a first monoclonal antibody specifically binding to a first epitope of nc-APRIL, wherein said first monoclonal antibody is an immobilized antibody;
   b) contacting the mixture of step (a) with a second monoclonal antibody, wherein said second monoclonal antibody specifically binds to a second epitope of nc-APRIL;
   c) detecting the binding of the second monoclonal antibody to immobilized nc-APRIL; and
   d) quantifying the concentration of nc-APRIL in the sample according to the detected binding in step (c).
22. A method for quantifying the total concentration of c-APRIL and nc-APRIL in a sample, the method comprising the steps of:
   a) contacting a denatured sample comprising nc-APRIL and/or c-APRIL with a first monoclonal antibody specifically binding to a first epitope of denatured nc-APRIL and c-APRIL, wherein said first monoclonal antibody is an immobilized antibody;
   b) contacting the mixture of step (a) with a second monoclonal antibody, wherein said second monoclonal antibody specifically binds to a second epitope of denatured nc-APRIL and c-APRIL;
   c) detecting the binding of the second monoclonal antibody to the immobilized forms of nc-APRIL and/or c-APRIL; and
   d) quantifying the total concentration of nc-APRIL and c-APRIL in the sample according to the detected binding in step (c).
23. A method for quantifying the amount of c-APRIL in a sample, the method comprising the steps of:
   a) quantifying the amount of nc-APRIL in a first portion of the sample with the method according to embodiment 21;
   b) quantifying the total amount of nc-APRIL and c-APRIL in a second portion of the sample with the method according to embodiment 22, wherein the second portion of the sample has been denatured; and
   c) quantifying the concentration of c-APRIL in the sample, wherein quantifying the concentration of c-APRIL in the sample involves subtracting the concentration of nc-APRIL in the sample from the total concentration of nc-APRIL and c-APRIL in the sample.
24. The method according to any one of embodiments 21 to 23, wherein the method is a sandwich enzyme-linked immunosorbent assay (ELISA) method.
25. The method according to any one of embodiments 21 to 24, wherein the second monoclonal antibody is conjugated to a detection moiety or a binding moiety.
26. The method according to embodiment 25, wherein the binding moiety is biotin.
27. The method according to any one of embodiments 21 to 26, wherein the first monoclonal antibody comprises a variable heavy (VH) chain sequence comprising the amino acid sequence of SEQ ID NO:56 or a sequence having 90%, preferably 95% sequence identity to SEQ ID NO:56; and a variable light (VL) chain sequence comprising the amino acid sequence of SEQ ID NO:60 or a sequence having 90%, preferably 95% sequence identity to SEQ ID NO:60; and wherein the second monoclonal antibody comprises
   (a) a variable heavy (VH) chain sequence comprising the amino acid sequence of SEQ ID NO:42, or a sequence having 90%, preferably 95% sequence identity to SEQ ID NO:42; and a variable light (VL) chain sequence comprising the amino acid sequence of SEQ ID NO:46, or a sequence having 90%, preferably 95% sequence identity to SEQ ID NO:46; or
   (b) a variable heavy (VH) chain sequence comprising the amino acid sequence of SEQ ID NO:49, or a sequence having 90%, preferably 95% sequence identity to SEQ ID NO:49; and a variable light (VL) chain sequence comprising the amino acid sequence of SEQ ID NO:53, or a sequence having 90%, preferably 95% sequence identity to SEQ ID NO:53.
28. The method according to any one of embodiments 21 to 26, wherein the first monoclonal antibody comprises
   (a) a variable heavy (VH) chain sequence comprising the amino acid sequence of SEQ ID NO:42, or a sequence having 90%, preferably 95% sequence identity to SEQ ID NO:42; and a variable light (VL) chain sequence comprising the amino acid sequence of SEQ ID NO:46, or a sequence having 90%, preferably 95% sequence identity to SEQ ID NO:46; or
   (b) a variable heavy (VH) chain sequence comprising the amino acid sequence of SEQ ID NO:49, or a sequence having 90%, preferably 95% sequence identity to SEQ ID NO:49; and a variable light (VL) chain sequence comprising the amino acid sequence of SEQ ID NO:53, or a sequence having 90%, preferably 95% sequence identity to SEQ ID NO:53; and
   wherein the second monoclonal antibody comprises a variable heavy (VH) chain sequence comprising the amino acid sequence of SEQ ID NO:56 or a sequence having 90%, preferably 95% sequence identity to SEQ ID NO:56; and a variable light (VL) chain sequence comprising the amino acid sequence of SEQ ID NO:60 or a sequence having 90%, preferably 95% sequence identity to SEQ ID NO:60
29. A kit for determining the level of nc-APRIL in a sample, the kit comprising a first and a second monoclonal antibody, wherein both monoclonal antibodies bind to different epitopes of nc-APRIL.
30. A nephelometric assay for quantifying the concentration of non-canonical APRIL (nc-APRIL) in a sample, the assay comprising the steps of:
   a) contacting a sample comprising nc-APRIL with at least one antibody, or antibody coupled to microparticles or microbeads specifically binding to nc-APRIL;
   b) transmitting light to the mixture of step (a);
   c) measuring a change in light scattering intensity of the mixture in response to the irradiation in step (b); and
   d) quantifying the concentration of nc-APRIL in the sample according to the measurement in step (c).
31. The method according to embodiment 30, wherein steps (a) to (c) are repeated with at least one dilution of the sample comprising nc-APRIL and/or the at least one antibody.
32. The method according to any one of embodiments 30 to 31, wherein the at least one antibody specifically binding to nc-APRIL comprises
   (a) a variable heavy (VH) chain sequence comprising the amino acid sequence of SEQ ID NO:42, or a sequence having 90%, preferably 95% sequence identity to SEQ ID NO:42; and a variable light (VL) chain sequence comprising the amino acid sequence of SEQ ID NO:46, or a sequence having 90%, preferably 95% sequence identity to SEQ ID NO:46; or
   (b) a variable heavy (VH) chain sequence comprising the amino acid sequence of SEQ ID NO:49, or a sequence having 90%, preferably 95% sequence identity to SEQ ID NO:49; and a variable light (VL) chain sequence comprising the amino acid sequence of SEQ ID NO:53, or a sequence having 90%, preferably 95% sequence identity to SEQ ID NO:53; or
   (c) a variable heavy (VH) chain sequence comprising the amino acid sequence of SEQ ID NO:56 or a sequence having 90%, preferably 95% sequence identity to SEQ ID NO:56; and a variable light (VL) chain sequence comprising the amino acid sequence of SEQ ID NO:60 or a sequence having 90%, preferably 95% sequence identity to SEQ ID NO:60
33. A method for predicting and/or diagnosing hypertriglyceridemia, metabolic syndrome, non-alcoholic steatohepatitis, diabetes mellitus type 2, atherogenic dyslipidemia, cardiovascular events and/or atherosclerosis, the method comprising the steps of:
   (a) determining the concentration of non-canonical APRIL (nc-APRIL) in a sample that has been obtained from said subject;
   (b) comparing the concentration of nc-APRIL that has been determined in step (a) to a reference value; and
   (c) predicting and/or diagnosing hypertriglyceridemia, metabolic syndrome, non-alcoholic steatohepatitis and diabetes mellitus type 2, atherogenic dyslipidemia, cardiovascular events and/or atherosclerosis in said subject based on the comparison made in step (b).
34. A method for predicting mortality risk in subjects suffering from hypertriglyceridemia, metabolic syndrome, non-alcoholic steatohepatitis, diabetes mellitus type 2, atherogenic dyslipidemia, cardiovascular events and/or atherosclerosis, the method comprising the steps of:
   (a) determining the concentration of nc-APRIL in a sample that has been obtained from said subject;
   (b) comparing the concentration of nc-APRIL that has been determined in step (a) to a reference value; and
   (c) determining the mortality risk of said subject based on the comparison made in step (b).
35. A method for determining whether a subject is susceptible to the treatment of hypertriglyceridemia, metabolic syndrome, non-alcoholic steatohepatitis, diabetes mellitus type 2, atherogenic dyslipidemia, cardiovascular events and/or atherosclerosis, the method comprising the steps of:
   (a) determining the concentration of nc-APRIL in two or more samples that have been obtained from said subject at an earlier and a later time point;
   (b) determining that said subject is susceptible to the treatment of hypertriglyceridemia, metabolic syndrome, non-alcoholic steatohepatitis and diabetes mellitus type 2, atherogenic dyslipidemia, cardiovascular events and/or atherosclerosis, if the concentration of nc-APRIL is higher in a sample that has been obtained at a later time point compared to a sample that has been obtained at an earlier time point; or determining that said subject is not susceptible to the treatment of hypertriglyceridemia, metabolic syndrome, non-alcoholic steatohepatitis, diabetes mellitus type 2, atherogenic dyslipidemia, cardiovascular events and/or atherosclerosis, if the concentration of nc-APRIL is similar or lower in a sample that has been obtained at a later time point compared to a sample that has been obtained at an earlier time point.
36. The method according to embodiment 35, wherein the earlier time point is before the beginning of the treatment and the later time point is after the beginning of the treatment; or wherein the earlier and the later time points are after the beginning of the treatment.
37. The method according to embodiments 35 or 36, wherein the treatment comprises the use of the antibody, or antigen-binding fragment thereof, of embodiments 11 to 15 or the pharmaceutical composition of embodiments 18 to 20.
38. The method according to any one of embodiments 33 to 37, wherein the cardiovascular events comprise myocardial infarction, stroke, peripheral artery disease, angina pectoris and/or urgent hospitalization for angina leading to revascularization.
39. The method according to any one of embodiments 33 to 38, wherein the sample is or comprises human serum or human plasma.
40. The method according to any one of embodiments 32 to 39, wherein the concentration of nc-APRIL is determined with at least one antibody specifically binding to nc-APRIL.
41. The kit according to embodiment 29 or the method for quantifying according to one of embodiments 21 to 27, wherein the first monoclonal antibody specifically binds to an epitope within the amino acid sequence SEQ ID NO:96 and/or the second monoclonal antibody specifically binds to an epitope within the amino acid sequence SEQ ID NO:64.
42. The kit according to embodiment 29 or the method for quantifying according to one of embodiments 21 to 26 or 28, wherein the first monoclonal antibody specifically binds to an epitope within the amino acid sequence SEQ ID NO:64 and/or the second monoclonal antibody specifically binds to an epitope within the amino acid sequence SEQ ID NO:96.
43. The nephelometric assay according to one of embodiments 30 to 32, wherein at least one antibody specifically binding to nc-APRIL binds to an epitope within the amino acid sequence SEQ ID NO:64 and/or at least one antibody specifically binding to nc-APRIL binds to an epitope within the amino acid sequence SEQ ID NO:96.
44. The method for predicting and/or diagnosing according to embodiment 40, wherein at least one antibody specifically binding to nc-APRIL binds to an epitope within the amino acid sequence SEQ ID NO:64 and/or at least one antibody specifically binding to nc-APRIL binds to an epitope within the amino acid sequence SEQ ID NO:96.
45. The method according to any one of embodiments 33 to 40, wherein the concentration of nc-APRIL is determined with the method according to any one of embodiments 21 or 41 to 42, the nephelometric assay according to one of embodiments 30 to 32 or 43, or the kit according to embodiment 29.
46. An antibody, or an antigen-binding fragment thereof, specifically binding to APRIL, wherein the antibody, or the antigen-binding fragment thereof, binds to an epitope within the amino acid sequence SEQ ID NO:64 or SEQ ID NO:96.
47. The antibody, or antigen-binding fragment thereof, according to embodiment 46 wherein the antibody, or the antigen-binding fragment thereof, binds to an epitope within the amino acid sequence SEQ ID NO:96.
48. The antibody, or antigen-binding fragment thereof, according to embodiment 46 wherein the antibody, or the antigen-binding fragment thereof, binds to an epitope within the amino acid sequence SEQ ID NO:64.

Accordingly, the invention relates to an antibody, or an antigen-binding fragment thereof, specifically binding to APRIL for use in the prevention and/or treatment of hypertriglyceridemia, metabolic syndrome, non-alcoholic steatohepatitis, diabetes mellitus type 2, atherogenic dyslipidemia, cardiovascular events and/or atherosclerosis.

That is, the present invention is based, at least in part, on the surprising discovery that APRIL which was known as a component of the immune system is involved in cardiovascular diseases and, in particular, symptoms and adverse events secondary to disease-related cardiovascular processes. Accordingly, it was surprisingly found that antibodies or antigen-binding fragments thereof specifically binding to APRIL can be used in the prevention and/or treatment of such diseases, in particular hypertriglyceridemia, metabolic syndrome, non-alcoholic steatohepatitis, diabetes mellitus type 2, atherogenic dyslipidemia, cardiovascular events and/or atherosclerosis.

The most commonly prescribed treatments for hypertriglyceridemia are lifestyle interventions, and a combination of fibrates and statins. The combination of these treatments increases the risk for side effects such as myopathy, rhabdomyolysis and treatment effect is insufficient in some cases (Peter H. Jones, Michael H. Davidson, Reporting rate of rhabdomyolysis with fenofibrate+statin versus gemfibrozil+any statin, The American Journal of Cardiology, Volume 95, Issue 1, 2005). These adverse effects of the treatment on skeletal muscles is believed to result from the inhibition of the cholesterol synthesis (Sakamoto, Kazuho, and Junko Kimura. “Mechanism of statin-induced rhabdomyolysis.” Journal of pharmacological sciences 123.4 (2013): 289-294). Without being bound to theory, the triglycerides lowering effect of an antibody, or an antigen-binding fragment thereof, specifically binding to APRIL does not result from altered production of triglyceride in the liver (FIG. 6) but rather from enhanced clearance and may therefore have less adverse effects on skeletal muscle and could increase effectiveness of the treatment, alone or in combination with other treatments.

The reduction of triglyceride levels (FIG. 4, Example 5) and cholesterol levels (Example 5) by an antibody of the invention, or an antigen-binding fragment thereof, specifically binding to APRIL is useful for use in the prevention and/or treatment of hypertriglyceridemia, metabolic syndrome, non-alcoholic steatohepatitis, diabetes mellitus type 2, atherogenic dyslipidemia, cardiovascular events and/or atherosclerosis.

Furthermore, April is involved in the binding to heparin (FIG. 20) and in the formation of an increased aortic diameter (FIG. 19), which is associated with artherosclerosis and abdominal aortic aneurysm amongst others. Therefore, binding to APRIL can be used for prevention and/or treatment of damaged arteries which is a hallmark of the pathologies of hypertriglyceridemia, metabolic syndrome, non-alcoholic steatohepatitis, diabetes mellitus type 2, atherogenic dyslipidemia, abdominal aortic aneurysm, cardiovascular events and/or atherosclerosis. Accordingly, the invention is at least in part based on the finding that binding to APRIL is surprisingly useful in the prevention and/or treatment of hypertriglyceridemia, metabolic syndrome, non-alcoholic steatohepatitis, diabetes mellitus type 2, atherogenic dyslipidemia, abdominal aortic aneurysm, cardiovascular events and/or atherosclerosis.

A person skilled in the art is able to screen for other antibodies, or an antigen-binding fragment thereof, specifically binding to APRIL to determine their suitability for use in the prevention and/or treatment of hypertriglyceridemia, metabolic syndrome, non-alcoholic steatohepatitis, diabetes mellitus type 2, atherogenic dyslipidemia, cardiovascular events and/or atherosclerosis.

In some embodiments, such a screening is achieved by repeated treatment (e.g., weekly administration) of atherogenic diet—fed (e.g., diet containing 0.2% cholesterol and/or 21% fat or 40% fat or 60% fat fed e.g., for 1 day, or 1 month or 1 year) mice (e.g., Ldlr−/− or wild type mice (e.g., C57BL/6, or BALB/c or 129S1) with one or more different anti-APRIL antibodies (e.g., antibodies of the invention, such as, 104 and/or 108) and an isotype control for a certain period of time (e.g., one month) to screen for delayed onset and/or reduction of hypertriglyceridemia. In some embodiments, such a screening comprises initiating the antibody treatment prior to or at the start of feeding mice with an atherogenic diet and the primary readout is delayed onset of hypertriglyceridemia. In some embodiments, such a screening comprises initiating the antibody treatment after (e.g., two weeks) the initiation of the atherogenic diet, when hypertriglyceridemia is established and the primary readout of the screening is reduction of hypertriglyceridemia.

Accordingly, the antibody, or an antigen-binding fragment thereof, of the invention, specifically binding to APRIL can be used in the prevention and/or treatment of adverse events secondary to disease-related cardiovascular process, in particular in the prevention and/or treatment of hypertriglyceridemia.

Triglycerides, LDL and VLDL are associated with impaired insulin secretion (Cynthia L. Kelpe, Lisa M. Johnson, Vincent Poitout, Increasing Triglyceride Synthesis Inhibits Glucose-Induced Insulin Secretion in Isolated Rat Islets of Langerhans: A Study Using Adenoviral Expression of Diacylglycerol Acyltransferase, Endocrinology, Volume 143, Issue 9, 1 Sep. 2002, Pages 3326-3332; Lee D H. Lipoproteins and β-Cell Functions: From Basic to Clinical Data. Diabetes Metab J. 2014; 38(4):274-277). Further, development of dyslipidemia is considered a harbinger of future diabetes (Goldberg, Ira J. “Diabetic dyslipidemia: causes and consequences.” The Journal of Clinical Endocrinology & Metabolism 86.3 (2001): 965-971).

Therefore, reduction of triglyceride levels (FIG. 4, Example 5) and cholesterol levels (Example 5) by an antibody of the invention, or an antigen-binding fragment thereof, specifically binding to APRIL can be beneficial for glucose homeostasis in diabetes mellitus type 2 and/or metabolic syndrome.

Diabetes mellitus type 2 and all components of the metabolic syndrome have been associated to chronic systemic inflammation (Santos, Adriana Carvalho, et al. “Decreased Circulating Levels of APRIL: Questioning Its Role in Diabetes.” (2015); Donath, M., Shoelson, S. Type 2 diabetes as an inflammatory disease. Nat Rev Immunol 11, 98-107 (2011); Cirillo, P., Y. Y. Sautin, J. Kanellis, D. H. Kang, L. Gesualdo, T. Nakagawa, and R. J. Johnson, Systemic inflammation, metabolic syndrome and progressive renal disease. Nephrol Dial Transplant, 2009. 24(5) 1384-7). An antibody, or an antigen-binding fragment thereof, specifically binding to APRIL may modulate cytokine pathways (Hahne M, Kataoka T, Schröter M, et al. APRIL, a new ligand of the tumor necrosis factor family, stimulates tumor cell growth. J Exp Med. 1998; 188(6):1185-1190) and immune cell tissue infiltration (FIG. 5) and have a synergistic effect on inflammation mediated components of diabetes mellitus type 2 and the metabolic syndrome.

Accordingly, an antibody of the invention, or an antigen-binding fragment thereof, specifically binding to APRIL can be used in the prevention and/or treatment of adverse events secondary to disease-related cardiovascular process, in particular in the prevention and/or treatment of diabetes mellitus type 2 and/or in the prevention and/or treatment of metabolic syndrome.

In certain embodiments the invention relates to the antibody, or antigen-binding fragment thereof, for use of the invention, wherein the hypertriglyceridemia is at least one selected from the group of hypertriglyceridemia in metabolic syndrome, hypertriglyceridemia in non-alcoholic steatohepatitis-related, hypertriglyceridemia in diabetes mellitus type 2, hypertriglyceridemia in atherogenic, cardiovascular events with a history of hypertriglyceridemia and atherosclerosis with hypertriglyceridemia.

Hypertriglyceridemia is associated to increased morbidity and mortality, especially for premature cardiovascular disease (CVD), in individuals suffering from metabolic syndrome and/or diabetes mellitus type 2 this risk is greatly increased (Resnick, Helaine E., and Barbara V. Howard. “Diabetes and cardiovascular disease.” Annual review of medicine 53.1 (2002): 245-267; Bonora, E., The metabolic syndrome and cardiovascular disease. Ann Med, 2006. 38(1), 64-80). Hypertriglyceridemia is a common lipid abnormality in persons with metabolic syndrome and type 2 diabetes, typically occurs in conjunction with low HDL levels and atherogenic small dense LDL particles and is associated with increased cardiovascular risk (Subramanian, Savitha, and Alan Chait. “Hypertriglyceridemia secondary to obesity and diabetes.” Biochimica et Biophysica Acta (BBA)-Molecular and Cell Biology of Lipids 1821.5 (2012): 819-825).

Further, current therapy of Hypertriglyceridemia is associated with side effects such as myopathy, rhabdomyolysis (e.g. induced by a statin, fibrates and/or life-style changes), which limits the applicability of therapy of Hypertriglyceridemia in particular in patients with diabetes or metabolic syndrome. Furthermore, current therapy of Hypertriglyceridemia is known to alter the effect of glucose lowering treatment, which limits the applicability of therapy of hypertriglyceridemia in particular in patients with diabetes or metabolic syndrome. Since an antibody of the invention, or an antigen-binding fragment thereof, specifically binding to APRIL has a different mechanism of action than current therapy of Hypertriglyceridemia, it is likely that these limitations do not occur and treatment effect may be in particularly beneficial in the treatment of hypertriglyceridemia in patients with metabolic syndrome and/or diabetes mellitus type 2 with an antibody of the invention, or an antigen-binding fragment thereof, specifically binding to APRIL.

Accordingly, an antibody of the invention, or an antigen-binding fragment thereof, specifically binding to APRIL can be used in the prevention and/or treatment of adverse events secondary to disease-related cardiovascular process, in particular in the prevention and/or treatment of hypertriglyceridemia in patients with metabolic syndrome and/or diabetes mellitus type 2.

One of the primary characteristics of non-alcoholic fatty liver disease (NAFLD) including non-alcoholic steatohepatitis is the accumulation of triglycerides in the liver (Marjot T, Moolla A, Cobbold J F, Hodson L, Tomlinson J W. Nonalcoholic Fatty Liver Disease in Adults: Current Concepts in Etiology, Outcomes, and Management. Endocr Rev. 2020). Statins are among the most frequent prescribed drugs to for conditions involving triglycerides, including non-alcoholic steatohepatitis (Oseini A M, Sanyal A J. Therapies in non-alcoholic steatohepatitis (NASH). Liver Int. 2017; 37 Suppl 1(Suppl 1):97-103.). However, hepatic adverse effects are one of the most commonly known adverse effects reported with statins (Jose J. Statins and its hepatic effects: Newer data, implications, and changing recommendations. J Pharm Bioallied Sci. 2016; 8(1):23-28), limiting their use in treatment of liver related conditions. These adverse hepatic effects may result from changes in the lipid component of the hepatocyte membrane, leading to an increase in its permeability with subsequent leakage of liver enzymes. Without being bound to theory, the triglycerides lowering effect of an antibody, or an antigen-binding fragment thereof, specifically binding to APRIL does not result from altered production of triglyceride in the liver (FIG. 6) but rather from enhanced clearance and may therefore have less adverse hepatic effects.

Non-alcoholic steatohepatitis can also include inflammatory processes in the liver (Chalasani, Naga, et al. “The diagnosis and management of nonalcoholic fatty liver disease: practice guidance from the American Association for the Study of Liver Diseases.” Hepatology 67.1 (2018): 328-357.). An antibody, or an antigen-binding fragment thereof, specifically binding to APRIL alters inflammation for example via cytokine pathways (Hahne M, Kataoka T, Schröter M, et al. APRIL, a new ligand of the tumor necrosis factor family, stimulates tumor cell growth. J Exp Med. 1998; 188(6):1185-1190) and immune cell tissue infiltration (FIG. 5) and may therefore have synergistic effects in the treatment of non-alcoholic steatohepatitis, alone or in combination with other treatments.

Accordingly, an antibody of the invention, or an antigen-binding fragment thereof, specifically binding to APRIL can be used in the prevention and/or treatment of adverse events secondary to disease-related cardiovascular process, in particular in the prevention and/or treatment of non-alcoholic steatohepatitis.

Medications that lower of triglycerides and cholesterol, such as statins, have shown some effect in prevention and/or treatment of atherogenic dyslipidemia and consequences thereof, such as atherosclerosis (Bozentowicz-Wikarek, Maria, et al. “Effectiveness of lipid-lowering therapy with statins for secondary prevention of atherosclerosis-guidelines vs. reality.” Pharmacological Reports 64.2 (2012): 377-385). The reduction of triglyceride levels (FIG. 4, Example 5) and cholesterol levels (Example 5) upon treatment with an antibody specifically binding to APRIL indicates, that atherogenic dyslipidemia and/or risk factors for atherosclerosis can be reduced with an antibody, or an antigen-binding fragment thereof, specifically binding to APRIL.

Atherogenic dyslipidemia and atherosclerosis are associated with inflammatory processes, which typically begin with an accumulation of white blood cells, mostly monocytes/macrophages, in the inner layers of the artery walls and progresses from there (Moore K J, Sheedy F J, Fisher E A. Macrophages in atherosclerosis: a dynamic balance. Nat Rev Immunol. 2013; 13(10):709-721.). This macrophage content in early atherosclerotic lesions was reduced in an animal model of adverse events secondary to disease-related cardiovascular process (FIG. 5). Atherosclerosis in the aortic root (FIG. 16) and in the thoracic aorta (FIG. 16) was reduced in an animal model for adverse events secondary to disease-related cardiovascular process. Further, APRIL was detected in human arteries by the inventors (Example 1), indicating that the results from the animal models are of translational value for the conditions in human.

Accordingly, an antibody of the invention, or an antigen-binding fragment thereof, specifically binding to APRIL can be used in the prevention and/or treatment of adverse events secondary to disease-related cardiovascular process, in particular in the prevention and/or treatment of atherosclerosis and/or in particular in the prevention and/or treatment of atherogenic dyslipidemia.

The presence of APRIL in human arteries, in particular in arteries with atherosclerotic plaques (Example 1) and the correlation of nc-APRIL (Example 13) with cardiovascular events and mortality indicate a key role of APRIL in adverse events secondary to disease-related cardiovascular process in humans, in particular cardiovascular events in humans.

Accordingly, an antibody of the invention, or an antigen-binding fragment thereof, specifically binding to APRIL can be used in the prevention and/or treatment of adverse events secondary to disease-related cardiovascular process, in particular in the prevention and/or treatment of cardiovascular events.

Accordingly, an antibody of the invention, or an antigen-binding fragment thereof, specifically binding to APRIL can be used in the prevention and/or treatment of adverse events secondary to disease-related cardiovascular process, in particular in the prevention and/or treatment of myocardial infarction.

Accordingly, an antibody of the invention, or an antigen-binding fragment thereof, specifically binding to APRIL can be used in the prevention and/or treatment of adverse events secondary to disease-related cardiovascular process, in particular in the prevention and/or treatment of stroke.

Accordingly, an antibody of the invention, or an antigen-binding fragment thereof, specifically binding to APRIL can be used in the prevention and/or treatment of adverse events secondary to disease-related cardiovascular process, in particular in the prevention and/or treatment of peripheral artery disease.

Accordingly, an antibody of the invention, or an antigen-binding fragment thereof, specifically binding to APRIL can be used in the prevention and/or treatment of adverse events secondary to disease-related cardiovascular process, in particular in the prevention and/or treatment of angina pectoris.

Accordingly, an antibody of the invention, or an antigen-binding fragment thereof, specifically binding to APRIL can be used in the prevention and/or treatment of adverse events secondary to disease-related cardiovascular process, in particular in the prevention and/or treatment of urgent hospitalization for angina leading to revascularization.

In certain embodiments, the invention relates to the antibody, or antigen-binding fragment thereof, of the invention for use in the treatment and or prevention of abdominal aortic aneurysm.

APRIL is involved in the formation of an increased aortic diameter (FIG. 19, Example 14), which can be considered as a model for abdominal aortic aneurysm.

Accordingly, the invention is at least in part based on the finding, that binding to APRIL is surprisingly useful in the treatment of abdominal aortic aneurysm.

Without being bound by theory, it is believed that chylomicrons, VLDL, and/or LPL interact with proteoglycans (PG), such as cell-bound PG or extracellular matrix PG. Furthermore, binding of LDL and/or ApoB-carrying lipoproteins to the extracellular space (e.g. subendothelial space) triggers atherosclerotic plaque formation.

In certain embodiments of the invention, binding of the antibody, or antigen-binding fragment thereof, to APRIL results in an increased interaction of APRIL with proteoglycans.

The inventors found that APRIL competes with the binding of LDL to PGs. Notably, LPL is also arrested in PGs on the capillary endothelium. When LPL is released into the circulation, it results in a more efficient decrease of plasma triglycerides (to a greater extent than endothelium-bound LPL) by digesting triglyceride-rich lipoproteins. The inventors surprisingly found that the metabolism (e.g. enzymatic clearance) of compounds involved in the progression of adverse events secondary to disease-related cardiovascular process, such as triglycerides, can be supported for example by enhancing the interaction between APRIL and PGs (FIG. 8, Example 4).

Accordingly, in one embodiment, the invention provides an antibody, or an antigen-binding fragment thereof, specifically binding to APRIL, wherein the binding of the antibody of the invention, or antigen-binding fragment thereof, to APRIL results in an increased interaction of APRIL with proteoglycans and which can surprisingly be used in the prevention and/or treatment of adverse events secondary to disease-related cardiovascular process, such as, hypertriglyceridemia, metabolic syndrome, non-alcoholic steatohepatitis, and diabetes mellitus type 2, atherogenic dyslipidemia, cardiovascular events and/or atherosclerosis.

In certain embodiments of the invention binding of the antibody, or antigen-binding fragment thereof, to APRIL results in an increased interaction of APRIL with arterial and vascular proteoglycans.

Arterial and vascular proteoglycans play a particular important role in adverse events secondary to disease-related cardiovascular process also via arresting LDL in the subendothelial space (Little P J, Ballinger M L, Osman N. Vascular wall proteoglycan synthesis and structure as a target for the prevention of atherosclerosis. Vasc Health Risk Manag. 2007; 3(1):117-124.). The inventors herein demonstrate that an antibody binding to APRIL reduced atherosclerosis in the aortic root (FIG. 16) and in the thoracic aorta (FIG. 16) in an animal model for adverse events secondary to disease-related cardiovascular process. Further, APRIL was detected in human arteries with or without atherosclerotic plaques by the inventors (Example 1), indicating that the results from the animal models are of translational value for the conditions in human. Accordingly, in a particular embodiment of the invention, an antibody, or an antigen-binding fragment thereof, specifically binding to APRIL, wherein the binding of the antibody or antigen-binding fragment thereof to APRIL results in an increased interaction of APRIL with arterial and vascular proteoglycans can surprisingly be used in the prevention and/or treatment of hypertriglyceridemia, metabolic syndrome, non-alcoholic steatohepatitis, and diabetes mellitus type 2, atherogenic dyslipidemia, cardiovascular events and/or atherosclerosis.

In certain embodiments of the invention binding of the antibody, or antigen-binding fragment thereof, to APRIL results in an increased interaction of APRIL with proteoglycans that are comprised in an extracellular matrix.

Macrophage-mediated proteolytic remodeling of the extracellular matrix is a critical process in adverse events secondary to disease-related cardiovascular process (Skjot-Arkil, Helene, et al. “Macrophage-mediated proteolytic remodeling of the extracellular matrix in atherosclerosis results in neoepitopes: a potential new class of biochemical markers.” Assay and drug development technologies 8.5 (2010): 542-552.) The inventors herein demonstrate that an antibody binding to APRIL reduces macrophage content in early atherosclerotic lesions (FIG. 5).

Accordingly, in a further embodiment of the invention, an antibody, or antigen-binding fragment thereof, specifically binding to APRIL, wherein the binding of the antibody or antigen-binding fragment thereof to APRIL results in an increased interaction of APRIL with proteoglycans, in particular arterial and vascular proteoglycans, wherein the proteoglycans are comprised in an extracellular matrix is provided, which can surprisingly be used in the prevention and/or treatment of adverse events secondary to disease-related cardiovascular process, such as, hypertriglyceridemia, metabolic syndrome, non-alcoholic steatohepatitis, and diabetes mellitus type 2, atherogenic dyslipidemia, cardiovascular events and/or atherosclerosis.

In certain embodiments of the invention, the binding of the antibody or antigen-binding fragment thereof to APRIL modulates the interaction of APRIL with at least one of its endogenous receptors.

Endogenous APRIL is mostly occupied by immune receptors and therefore unavailable to support metabolism of compounds involved in the progression of adverse events secondary to disease-related cardiovascular process. The inventors demonstrated that antibodies binding to APRIL can modulate the binding to endogenous receptors (FIG. 3, Example 4).

Accordingly, in a particular embodiment of the invention, an antibody, or antigen-binding fragment thereof, specifically binding to APRIL, wherein the binding of the antibody or antigen-binding fragment thereof to APRIL, wherein the binding of the antibody of the invention or antigen-binding fragment thereof to APRIL modulates the interaction of APRIL with at least one of its endogenous receptors is provided, which can surprisingly be used in the prevention and/or treatment of adverse events secondary to disease-related cardiovascular process, such as, hypertriglyceridemia, metabolic syndrome, non-alcoholic steatohepatitis, and diabetes mellitus type 2, atherogenic dyslipidemia, cardiovascular events and/or atherosclerosis.

In certain embodiments of the invention, the binding of the antibody or antigen-binding fragment thereof to APRIL blocks the interaction of APRIL with the receptors TACI and BCMA.

The documents Mackay, F. & Schneider, P. Cracking the BAFF code. Nat Rev Immunol 9, 491-502 (2009), Castigli, E., et al. Impaired IgA class switching in APRIL-deficient mice. Proc Natl Acad Sci USA 101, 3903-3908 (2004), Vincent, F. B., Morand, E. F., Schneider, P. & Mackay, F. The BAFF/APRIL system in SLE pathogenesis. Nat Rev Rheumatol 10, 365-373 (2014) disclose that APRIL binds to immune receptors such as BCMA and/or TAC. However, these documents do not disclose that binding to these receptors is involved in the metabolism of compounds involved in the progression of adverse events secondary to disease-related cardiovascular process, such as triglycerides. The inventors found that an antibody of the invention or an antigen-binding fragment thereof, specifically binding to APRIL that modulates (e.g., blocks) the interaction of APRIL with its endogenous receptors such as the receptors TACI and BCMA would reduce the occupation of endogenous APRIL by immune receptors. Accordingly, the antibody, or antigen-binding fragment thereof, of the invention, specifically binding to APRIL, wherein the binding of the antibody or antigen-binding fragment thereof to APRIL blocks the interaction of APRIL with the receptors TACI and BCMA can surprisingly be used in the prevention and/or treatment of adverse events secondary to disease-related cardiovascular process, such as, hypertriglyceridemia, metabolic syndrome, non-alcoholic steatohepatitis, and diabetes mellitus type 2, atherogenic dyslipidemia, cardiovascular events and/or atherosclerosis.

In certain embodiments of the invention, the antibody, or antigen-binding fragment thereof, specifically binding to APRIL, as described herein, comprises a variable heavy (VH) chain comprising CDR3 as defined in SEQ ID NO:10 and a variable light (VL) chain comprising CDR3 as defined in SEQ ID NO:13; In certain embodiments of the invention, the antibody, or antigen-binding fragment thereof, specifically binding to APRIL, as described herein, comprises a variable heavy (VH) chain comprising CDR3 as defined in SEQ ID NO:17 and a variable light (VL) chain comprising CDR3 as defined in SEQ ID NO:20.

In certain embodiments of the invention, the antibody, or antigen-binding fragment thereof, specifically binding to APRIL, as described herein, comprises a variable heavy (VH) chain comprising CDR3 as defined in SEQ ID NO:24 and a variable light (VL) chain comprising CDR3 as defined in SEQ ID NO:27.

In certain embodiments of the invention, the antibody, or antigen-binding fragment thereof, specifically binding to APRIL, as described herein, comprises a variable heavy (VH) chain comprising CDR3 as defined in SEQ ID NO:31 and a variable light (VL) chain comprising CDR3 as defined in SEQ ID NO:34.

In certain embodiments of the invention, the antibody, or antigen-binding fragment thereof, specifically binding to APRIL, as described herein, comprises a variable heavy (VH) chain comprising CDR3 as defined in SEQ ID NO:38 and a variable light (VL) chain comprising CDR3 as defined in SEQ ID NO:41.

In certain embodiments of the invention, the antibody described herein, or antigen-binding fragment thereof, specifically binding to APRIL, as described herein, comprises a variable heavy (VH) chain comprising CDR1 as defined in SEQ ID NO:8, CDR2 as defined in SEQ ID NO:9 and CDR3 as defined in SEQ ID NO:10 and a variable light (VL) chain comprising CDR1 as defined in SEQ ID NO:12, CDR2 as defined in the sequence: YAS and CDR3 as defined in SEQ ID NO:13.

In certain embodiments of the invention, the antibody, or antigen-binding fragment thereof, specifically binding to APRIL, as described herein, comprises a variable heavy (VH) chain comprising CDR1 as defined in SEQ ID NO:15, CDR2 as defined in SEQ ID NO:16 and CDR3 as defined in SEQ ID NO:17 and a variable light (VL) chain comprising CDR1 as defined in SEQ ID NO:19, CDR2 as defined by the amino acid sequence: AAS and CDR3 as defined in SEQ ID NO:20.

In certain embodiments of the invention, the antibody, or antigen-binding fragment thereof, specifically binding to APRIL, as described herein, comprises a variable heavy (VH) chain comprising CDR1 as defined in SEQ ID NO:22, CDR2 as defined in SEQ ID NO:23 and CDR3 as defined in SEQ ID NO:24 and a variable light (VL) chain comprising CDR1 as defined in SEQ ID NO:26, CDR2 as defined by the amino acid sequence: GTN and CDR3 as defined in SEQ ID NO:27.

In certain embodiments of the invention, the antibody, or antigen-binding fragment thereof, specifically binding to APRIL, as described herein, comprises a variable heavy (VH) chain comprising CDR1 as defined in SEQ ID NO:29, CDR2 as defined in SEQ ID NO:30 and CDR3 as defined in SEQ ID NO:31 and a variable light (VL) chain comprising CDR1 as defined in SEQ ID NO:33, CDR2 as defined by the amino acid sequence: GTS and CDR3 as defined in SEQ ID NO:34.

In certain embodiments of the invention, the antibody, or antigen-binding fragment thereof, specifically binding to APRIL, as described herein, comprises a variable heavy (VH) chain comprising CDR1 as defined in SEQ ID NO:36, CDR2 as defined in SEQ ID NO:37 and CDR3 as defined in SEQ ID NO:38 and a variable light (VL) chain comprising CDR1 as defined in SEQ ID NO:40, CDR2 as defined by the amino acid sequence LVS and CDR3 as defined in SEQ ID NO:41.

The antibodies Aprily 1, Apry 1.1, Aprily 2, Aprily 5, 104, 108, 110, 115, 2C8 specifically bind to APRIL and all tested antibodies (Apry 1.1, 104, 108, 110, 115, 2C8) binding to APRIL increase the interaction between PG and APRIL (FIG. 8). This can be a result of binding to the BCMA/TACI-binding site of APRIL as exemplified by Apry 1.1 (FIG. 3) or by other epitopes in close proximity with the BCMA/TACI binding site as exemplified by 104 (Example 9).

In certain embodiments of the invention, antibodies (as exemplified by Apry 1.1), or an antigen-binding fragment thereof, specifically binding to APRIL, can result in a decrease of markers of disease progression of adverse events secondary to disease-related cardiovascular process such as triglyceride levels (FIG. 4, Example 5), cholesterol levels (Example 5), macrophage content in early atherosclerotic lesions (FIG. 5) and atherosclerosis in the aortic root and in the thoracic aorta (FIG. 16), while body weight and liver triglyceride production remains unaffected (FIG. 6).

Accordingly, antibodies, or antigen-binding fragments thereof, comprising a VH chain comprising a CDR3 as described herein and a VL chain comprising a CDR3 as described herein can be used in the invention.

Accordingly, antibodies, or antigen-binding fragments thereof, comprising a VH chain comprising the CDR1, CDR2 and CDR3 as described herein and a VL chain comprising a CDR1, CDR2 and CDR3 as described herein can be used in the invention.

In certain embodiments of the invention, the antibody described herein, or antigen-binding fragment thereof described herein, (a) comprises a variable heavy (VH) chain sequence comprising the amino acid sequence of SEQ ID NO:7 or a sequence having 90%, preferably 95% sequence identity to SEQ ID NO:7; and a variable light (VL) chain sequence comprising the amino acid sequence of SEQ ID NO:11 or a sequence having 90%, preferably 95% sequence identity to SEQ ID NO:11; or (b) comprises a variable heavy (VH) chain sequence comprising the amino acid sequence of SEQ ID NO:14 or a sequence having 90%, preferably 95% sequence identity to SEQ ID NO:14; and a variable light (VL) chain sequence comprising the amino acid sequence of SEQ ID NO:18 or a sequence having 90%, preferably 95% sequence identity to SEQ ID NO:18; (c) comprises a variable heavy (VH) chain sequence comprising the amino acid sequence of SEQ ID NO:21 or a sequence having 90%, preferably 95% sequence identity to SEQ ID NO:21; and a variable light (VL) chain sequence comprising the amino acid sequence of SEQ ID NO:25 or a sequence having 90%, preferably 95% sequence identity to SEQ ID NO:25; (d) comprises a variable heavy (VH) chain sequence comprising the amino acid sequence of SEQ ID NO:28 or a sequence having 90%, preferably 95% sequence identity to SEQ ID NO:28; and a variable light (VL) chain sequence comprising the amino acid sequence of SEQ ID NO:32 or a sequence having 90%, preferably 95% sequence identity to SEQ ID NO:32; or (e) comprises a variable heavy (VH) chain sequence comprising the amino acid sequence of SEQ ID NO:35 or a sequence having 90%, preferably 95% sequence identity to SEQ ID NO:35; and a variable light (VL) chain sequence comprising the amino acid sequence of SEQ ID NO:39 or a sequence having 90%, preferably 95% sequence identity to SEQ ID NO:39.

In certain embodiments of the invention, an antibody, or antigen-binding fragment thereof, specifically binding to APRIL, comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:7, SEQ ID NO: 14, SEQ ID NO:21, SEQ ID NO:28 or SEQ ID NO:35. In certain embodiments, a VH sequence having at least 90%, 91%, 92%, 93%, 94%, 95%, 96%, 97%, 98%, or 99% identity to the amino acid sequence of SEQ ID NO:7, SEQ ID NO:14, SEQ ID NO:21, SEQ ID NO:28 or SEQ ID NO:35 contains substitutions (e.g., conservative substitutions), insertions, or deletions relative to the reference sequence, but an antibody of the invention, or antigen-binding fragment thereof, specifically binding to APRIL, comprising that sequence retains the ability to specifically bind to APRIL. In certain embodiments, a total of 1 to 10 amino acids have been substituted, inserted and/or deleted in SEQ ID NO:7, SEQ ID NO:14, SEQ ID NO:21, SEQ ID NO:28 or SEQ ID NO:35. In certain embodiments, a total of 1 to 5 amino acids have been substituted, inserted and/or deleted in SEQ ID NO:7, SEQ ID NO:14, SEQ ID NO:21, SEQ ID NO:28 or SEQ ID NO:35. In certain embodiments, substitutions, insertions, or deletions occur in regions outside the CDRs (i.e., in the FRs). In a preferred embodiment, a total of 10 amino acids in SEQ ID NO:7, SEQ ID NO:14, SEQ ID NO:21, SEQ ID NO:28 or SEQ ID NO:35 have been substituted to optimize the expression in mammalian cells. Optionally, an antibody of the invention, or antigen-binding fragment thereof, specifically binding to APRIL, comprises the VH sequence of SEQ ID NO:7, SEQ ID NO:14, SEQ ID NO:21, SEQ ID NO:28 or SEQ ID NO:35, including post-translational modifications of that sequence.

In another aspect, an antibody, or antigen-binding fragment thereof, specifically binding to APRIL, is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:11, SEQ ID NO:18, SEQ ID NO:25, SEQ ID NO:32 or SEQ ID NO:39. In certain embodiments, a VL sequence having at least 90%, 91%, 92%, 93%, 94%, 95%, 96%, 97%, 98%, or 99% identity to the amino acid sequence of SEQ ID NO:11, SEQ ID NO:18, SEQ ID NO:25, SEQ ID NO:32 or SEQ ID NO:39 contains substitutions (e.g., conservative substitutions), insertions, or deletions relative to the reference sequence, but an antibody of the invention, or antigen-binding fragment thereof, specifically binding to APRIL, comprising that sequence retains the ability to specifically bind to APRIL. In certain embodiments, a total of 1 to 10 amino acids have been substituted, inserted and/or deleted in SEQ ID NO: 11, SEQ ID NO:18, SEQ ID NO:25, SEQ ID NO:32 or SEQ ID NO:39. In certain embodiments, a total of 1 to 5 amino acids have been substituted, inserted and/or deleted in SEQ ID NO: 11, SEQ ID NO:18, SEQ ID NO:25, SEQ ID NO:32 or SEQ ID NO:39 In certain embodiments, the substitutions, insertions, or deletions occur in regions outside the CDRs (i.e., in the FRs). In a preferred embodiment, a total of 10 amino acids in SEQ ID NO:46, SEQ ID NO:53, SEQ ID NO:60, SEQ ID NO:11, SEQ ID NO:18, SEQ ID NO:25, SEQ ID NO:32 or SEQ ID NO:39 have been substituted to optimize the expression in mammalian cells. Optionally, the antibody of the invention, or antigen-binding fragment thereof, specifically binding to APRIL, comprises the VL sequence of SEQ ID NO:11, SEQ ID NO:18, SEQ ID NO:25, SEQ ID NO:32 or SEQ ID NO:39, including post-translational modifications of that sequence.

In another aspect, an antibody, or antigen-binding fragment thereof, specifically binding to APRIL, is provided, wherein the antibody comprises a VH as in any of the embodiments provided above, and a VL as in any of the embodiments provided above. In a preferred embodiment, the antibody comprises the VH and VL sequences in SEQ ID NO:7 and SEQ ID NO:11 or SEQ ID NO:14 and SEQ ID NO:18 or SEQ ID NO:21 and SEQ ID NO:25 or SEQ ID NO:28 and SEQ ID NO:32 or SEQ ID NO:35 and SEQ ID NO:39, respectively, including post-translational modifications of those sequences. In another embodiment, an antibody, or antigen-binding fragment thereof, specifically binding to APRIL, comprises a humanized form of an antibody comprising the VH and VL sequences in or SEQ ID NO:7 and SEQ ID NO:11 or SEQ ID NO:14 and SEQ ID NO:18 or SEQ ID NO:21 and SEQ ID NO:25 or SEQ ID NO:28 and SEQ ID NO:32 or SEQ ID NO:35 and SEQ ID NO:39, respectively. In one embodiment, the antibody comprises the VH and VL sequences in or SEQ ID NO:7 and SEQ ID NO:11 or SEQ ID NO:14 and SEQ ID NO:18 or SEQ ID NO:21 and SEQ ID NO:25 or SEQ ID NO:28 and SEQ ID NO:32 or SEQ ID NO:35 and SEQ ID NO:39, respectively, including post-translational modifications of those sequences.

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling.

In certain embodiments, substitutions, insertions, or deletions may occur within one or more CDRs so long as such alterations do not substantially reduce the ability of the antibody of the invention to bind antigen. For example, conservative alterations that do not substantially reduce binding affinity may be made in CDRs. Such alterations may be outside of CDR “hotspots” or SDRs. In certain embodiments of the variant VH and VL sequences provided above, each CDR either is unaltered or contains no more than one, two or three amino acid substitutions.

In certain embodiments of the invention, the antibody, specifically binding to APRIL, as described herein, is an IgM, IgG1, IgG2a or IgG2b, IgG3, IgG4, IgA or IgE antibody.

In certain embodiments of the invention, the antibody, or the antigen-binding fragment thereof, specifically binding to APRIL, as described herein, is a Fab fragment, an F(ab′) fragment, an Fv fragment or an scFv fragment.

Accordingly, in the context of the present invention, the antibody, or antigen-binding fragment thereof specifically binding to APRIL described hereinabove is selected from the group consisting of a full antibody (immunoglobulin, like an IgM, IgG1, IgG2a or IgG2b, IgG3, IgG4, IgA or IgE), Fab′-SH-, Fab fragment, an F(ab′) fragment, an Fv fragment, an scFv fragment, a chimeric antibody, a CDR-grafted antibody, a fully human antibody, a bivalent antibody-construct, an antibody-fusion protein, a synthetic antibody, bivalent single-chain antibody, a trivalent single chain antibody and a multivalent single-chain antibody.

In some embodiments, the antibody or antigen binding fragment thereof, described herein refers to the antibody or antigen binding fragment thereof, for use in prevention and/or at least one treatment of an indication described herein.

In certain embodiments, the invention relates to a polynucleotide encoding an antibody, or an antigen-binding fragment thereof, specifically binding to APRIL as described herein. In a particular embodiment, the polynucleotide of the invention encodes at least one variable heavy (VH) chain sequence and/or at least one variable light (VL) chain sequence of an antibody of the invention that specifically binds APRIL, preferably to an epitope at least in part within the amino acid sequence SEQ ID NO:96 or within the amino acid sequence SEQ ID NO:64.

In some embodiments the polynucleotide of the invention encoding at least one variable heavy (VH) chain sequence of an antibody of the invention that specifically binds APRIL comprises the nucleotide sequence as defined by SEQ ID NO:65.

In some embodiments the polynucleotide of the invention encoding at least one variable light (VL) chain sequence of an antibody of the invention that specifically binds APRIL comprises the nucleotide sequence as defined by SEQ ID NO:66.

In some embodiments the polynucleotide of the invention encoding at least one variable heavy (VH) chain sequence of an antibody of the invention that specifically binds APRIL comprises the nucleotide sequence as defined by SEQ ID NO:67.

In some embodiments the polynucleotide of the invention encoding at least one variable light (VL) chain sequence of an antibody of the invention that specifically binds APRIL comprises the nucleotide sequence as defined by SEQ ID NO:68.

In some embodiments the polynucleotide of the invention encoding at least one variable heavy (VH) chain sequence of an antibody of the invention that specifically binds APRIL comprises the nucleotide sequence as defined by SEQ ID NO:69.

In some embodiments the polynucleotide of the invention encoding at least one variable light (VL) chain sequence of an antibody of the invention that specifically binds APRIL comprises the nucleotide sequence as defined by SEQ ID NO:70.

In some embodiments the polynucleotide of the invention encoding at least one variable heavy (VH) chain sequence of an antibody of the invention that specifically binds APRIL comprises the nucleotide sequence as defined by SEQ ID NO:71.

In some embodiments the polynucleotide of the invention encoding at least one variable light (VL) chain sequence of an antibody of the invention that specifically binds APRIL comprises the nucleotide sequence as defined by SEQ ID NO:72.

In some embodiments the polynucleotide of the invention encoding at least one variable heavy (VH) chain sequence of an antibody of the invention that specifically binds APRIL comprises the nucleotide sequence as defined by SEQ ID NO:73.

In some embodiments the polynucleotide of the invention encoding at least one variable light (VL) chain sequence of an antibody of the invention that specifically binds APRIL comprises the nucleotide sequence as defined by SEQ ID NO:74.

In some embodiments the polynucleotide of the invention encoding at least one variable heavy (VH) chain sequence of an antibody of the invention that specifically binds APRIL comprises the nucleotide sequence as defined by SEQ ID NO:75.

In some embodiments the polynucleotide of the invention encoding at least one variable light (VL) chain sequence of an antibody of the invention that specifically binds APRIL comprises the nucleotide sequence as defined by SEQ ID NO:76.

In some embodiments the polynucleotide of the invention encoding at least one variable heavy (VH) chain sequence of an antibody of the invention that specifically binds APRIL comprises the nucleotide sequence as defined by SEQ ID NO:77.

In some embodiments the polynucleotide of the invention encoding at least one variable light (VL) chain sequence of an antibody of the invention that specifically binds APRIL comprises the nucleotide sequence as defined by SEQ ID NO:78.

In some embodiments the polynucleotide of the invention encoding at least one variable heavy (VH) chain sequence of an antibody of the invention that specifically binds APRIL comprises the nucleotide sequence as defined by SEQ ID NO:79.

In some embodiments the polynucleotide of the invention encoding at least one variable light (VL) chain sequence of an antibody of the invention that specifically binds APRIL comprises the nucleotide sequence as defined by SEQ ID NO:80.

In some embodiments, the polynucleotide described herein refers to the polynucleotide for use in prevention and/or at least one treatment of an indication described herein.

The invention furthermore relates to a host cell comprising the polynucleotide of the invention. The host cell comprising the polynucleotide of the invention may be used in treatment (e.g. gene therapy) and or for the production of biologic entities such as antibodies. Furthermore, the invention relates to a method of producing an antibody of the invention comprising culturing the host cell of the invention, wherein the host cell comprises the polynucleotide of the invention. In a particular embodiment, the method of producing an antibody comprises culturing the host cell of the invention under conditions suitable to allow efficient production of the antibody of the invention.

The present invention also relates to the production of specific antibodies binding to native polypeptides and recombinant polypeptides of APRIL, in particular of nc-APRIL. This production is based, for example, on the immunization of animals, like mice. However, also other animals for the production of antibody/antisera are envisaged within the present invention. For example, monoclonal and polyclonal antibodies can be produced by rabbit, mice, goats, donkeys and the like. The polynucleotide encoding a correspondingly chosen polypeptide of APRIL can be subcloned into an appropriated vector, wherein the recombinant polypeptide is to be expressed in an organism being able for an expression, for example in bacteria. Thus, the expressed recombinant protein can be intra-peritoneally injected into a mouse and the resulting specific antibody can be, for example, obtained from the mice serum being provided by intra-cardiac blood puncture. The present invention also envisages the production of specific antibodies against native polypeptides and recombinant polypeptides by using a DNA vaccine strategy. DNA vaccine strategies are well-known in the art and encompass liposome-mediated delivery, by gene gun or jet injection and intramuscular or intradermal injection. Thus, antibodies directed against a polypeptide or an epitope of APRIL, in particular the epitope of the antibodies provided herein, can be obtained by directly immunizing the animal by directly injecting intramuscularly the vector expressing the desired polypeptide or an epitope of APRIL, in particular, the epitope of the antibodies of the invention, which lies at least in part within the amino acid sequence SEQ ID NO:96 or at least in part within the amino acid sequence SEQ ID NO:64, preferably within the amino acid sequence SEQ ID NO:96 or preferably within the amino acid sequence SEQ ID NO:64. The amount of obtained specific antibody can be quantified using an ELISA, which is also described hereinbelow. Further methods for the production of antibodies are well known in the art, see, e.g., Harlow and Lane, “Antibodies, A Laboratory Manual”, CSH Press, Cold Spring Harbor, 1988.

In certain embodiments, the invention relates to a pharmaceutical composition comprising the antibody, or an antigen-binding fragment thereof, specifically binding to APRIL as described herein and a pharmaceutically acceptable carrier.

In some embodiments, the pharmaceutical composition described herein refers to the pharmaceutical composition for use in prevention and/or at least one treatment of an indication described herein.

Pharmaceutical compositions of an antibody, or an antigen-binding fragment thereof, specifically binding to APRIL as described herein are prepared by mixing such antibody or an antigen-binding fragment thereof, having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody or antigen-binding fragment compositions are described in U.S. Pat. No. 6,267,958. Aqueous antibody or antigen-binding fragment compositions include those described in U.S. Pat. No. 6,171,586 and WO2006/044908, the latter formulations including a histidine-acetate buffer.

Sustained-release preparations may be prepared. Suitable examples of sustained-release preparations include semipermeable matrices of solid hydrophobic polymers containing the antibody or an antigen-binding fragment thereof, specifically binding to APRIL, which matrices are in the form of shaped articles, e.g., films, or microcapsules. The compositions to be used for in vivo administration are generally sterile. Sterility may be readily accomplished, e.g., by filtration through sterile filtration membranes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In certain embodiments, the invention relates to a pharmaceutical composition as described herein comprising the antibody of the invention, or an antigen-binding fragment thereof, specifically binding to APRIL and additionally comprising a further therapeutic agent.

The pharmaceutical composition herein may also contain a further therapeutic agent as necessary for the particular indication being treated, preferably those with complementary activities that do not adversely affect each other.

In certain embodiments of the invention, the further therapeutic agent is at least one drug moiety and is/are linked to the antibody of the invention, or an antigen-binding fragment thereof, specifically binding to APRIL to form an antibody-drug conjugate. Antibody-drug conjugates are targeted therapeutic molecules that combine properties of both antibodies and drugs by targeting potent cytotoxic drugs to antigen-expressing tissue (e.g., blood vessels), thereby enhancing the therapeutic index by maximizing efficacy and minimizing off-target toxicity (e.g., hepatotoxicity).

In certain embodiments of the invention, the further therapeutic agent is at least one antibody part combined with the antibody of the invention, or an antigen-binding fragment thereof, specifically binding to APRIL to form at least one multispecific (e.g., bispecific) antibody.

A multispecific antibody may have an enhanced therapeutic index and may reduce the number of needed applications compared to multiple (e.g., two) monospecific antibodies.

In certain embodiments of the invention, the further therapeutic agent is a therapeutic agent with properties that reduce cholesterol, blood pressure (Angiotensin-converting enzyme (ACE) inhibitors), blood glucose (e.g., insulin), body weight (e.g., GLP-1-Receptor agonists), chronic low-grade inflammation (e.g., anti-IL-1beta), risk of blood clotting (e.g., cyclooxygenase inhibitors) and/or nicotine craving (e.g., nicotine receptor agonists)

In certain embodiments, the invention relates to the pharmaceutical composition as described herein comprising the antibody of the invention, or an antigen-binding fragment thereof, specifically binding to APRIL and a further therapeutic agent selected from a group consisting of: fibrates, statins, agents that inhibit the expression of Angiopoietin-like protein 3 (ANGPTL3) or Apolipoprotein C-III (apoC-III), and agents that prevent the binding of auto-antibodies to GPIHBP1.

The pharmaceutical composition herein may contain a further therapeutic agent selected from a group consisting of fibrates, statins, agents that inhibit the expression of Angiopoietin-like protein 3 (ANGPTL3) or Apolipoprotein C-III (apoC-III), and agents that prevent the binding of auto-antibodies to GPIHBP1. Therefore, the further therapeutic agent may specifically decrease LDL alone to support, preferably to synergistically improve, the therapeutically beneficial effect of the antibody, or an antigen-binding fragment thereof, specifically binding to APRIL as described herein.

Any of the antibodies, or an antigen-binding fragment thereof, provided herein may be used in methods, e.g., therapeutic or analytical methods.

The inventors surprisingly discovered an additional novel form of APRIL that is present e.g., in human serum and plasma. The novel form of APRIL, herein referred to as “non-canonical APRIL” or “nc-APRIL”, displays distinct binding properties and can have distinct amino acid sequences (FIG. 18) compared to the known form of APRIL, herein referred-to as “canonical APRIL” or “c-APRIL”.

Invitrogen and Adipogen offer kits to measure human APRIL (Adipogen REF: AG-45B-0012-KI01, Invitrogen Human APRIL ELISA Kit, REF: BMS2008). These kits are only capable of detecting one of the APRIL forms (nc-APRIL or c-APRIL (FIG. 10, 11)). Provided herein are the means and methods to detect all forms of APRIL. Further, the inventors found, that the commercially available kits have a low sensitivity, require larger sample amounts, require more washing repetitions and are relatively time intense for certain application compared to the method of the invention (FIG. 10, FIG. 17, Example 11).

In certain embodiments of the invention, a method for quantifying the concentration of non-canonical APRIL (nc-APRIL) in a sample, the method comprising the steps of: a) contacting the sample comprising nc-APRIL with a first monoclonal antibody specifically binding to a first epitope of nc-APRIL, wherein said first monoclonal antibody is an immobilized antibody; b) contacting the mixture of step (a) with a second monoclonal antibody, wherein said second monoclonal antibody specifically binds to a second epitope of nc-APRIL; c) detecting the binding of the second monoclonal antibody to immobilized nc-APRIL; and d) quantifying the concentration of nc-APRIL in the sample according to the detected binding in step (c).

The detection of the binding of the second monoclonal antibody to immobilized nc-APRIL can be achieved in various ways, e.g., using a binding moiety or a detection moiety, such as, a label. Labels include, but are not limited to, labels or moieties that are detected directly (such as fluorescent, chromophoric, electron-dense, chemiluminescent, electrochemiluminescence and radioactive labels), as well as moieties, such as enzymes or ligands, that are detected indirectly, e.g., through an enzymatic reaction or molecular interaction.

Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, ß-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, stable free radicals, and the like. In another embodiment, a label is a positron emitter. Positron emitters include but are not limited to ⁶⁸Ga, ¹⁸F, ⁶⁴Cu, ⁸⁶Y, ⁷⁶Br, ⁸⁹Zr, and ¹²⁴I.

In a certain embodiment of the invention, the detection of the binding of the second monoclonal antibody to immobilized nc-APRIL can be achieved in that the second monoclonal antibody allows binding of an additional binding entity (e.g., third antibody) that comprises a binding moiety or a detection moiety (e.g., a label).

In a certain embodiment of the invention, the method may be an immunoradiometric assay and the detection of the binding of the second monoclonal antibody to immobilized nc-APRIL comprise the use of a radioactive label.

In a certain embodiment of the invention, the method may be a flow cytometry assay and the first monoclonal antibody is immobilized on the surface of a cell, cell part, or particle (e.g., bead), whereas the second monoclonal antibody may be conjugated to a binding moiety or a detection moiety (e.g., a label).

In certain embodiments of the invention, the method is a magnetic sandwich immunofiltration assay and the second monoclonal antibody is bound to a magnetic moiety (e.g. magnetic bead) or the second monoclonal antibody allows binding of an additional binding entity (e.g. third monoclonal antibody) that comprises a magnetic moiety (e.g. magnetic bead).

The inventors developed a novel method, for example, an ELISA method, for quantifying nc-APRIL, such as using two antibodies that specifically bind to different epitopes of nc-APRIL. The invented method only requires a sample amount that can be up to 40 times smaller compared to commercially available human APRIL Kits (Invitrogen Human APRIL ELISA Kit, REF: BMS2008). Further, the invented method and/or some steps of the invented method require less time (FIG. 17, Example 11 and 12). The novel method can, therefore, save working resources compared to a commercially available human APRIL Kit. Further, the invented method can be more sensitive with a lower lowest limit of detection and/or a lower lowest limit of quantification (FIG. 17, Example 12). Accordingly, the surprising effect of the method of the invention is in part based on the surprising efficiency, sensitivity and specificity that can be achieved by using two antibodies binding to different epitopes of APRIL.

In a certain embodiment of the invention, a method for quantifying the total concentration of c-APRIL and nc-APRIL in a sample, the method comprising the steps of: a) contacting a denatured sample comprising nc-APRIL and/or c-APRIL with a first monoclonal antibody specifically binding to a first epitope of denatured nc-APRIL and c-APRIL, wherein said first monoclonal antibody is an immobilized antibody; b) contacting the mixture of step (a) with a second monoclonal antibody, wherein said second monoclonal antibody specifically binds to a second epitope of denatured nc-APRIL and c-APRIL; c) detecting the binding of the second monoclonal antibody to the immobilized forms of nc-APRIL and/or c-APRIL; and d) quantifying the total concentration of nc-APRIL and c-APRIL in the sample according to the detected binding in step (c).

Accordingly, the novel method for quantifying total APRIL is using two antibodies that specifically bind to different epitopes of denatured nc-APRIL and c-APRIL respectively. The protocols of commercially available human APRIL Kits (Invitrogen Human APRIL ELISA Kit, REF: BMS2008, Adipogen REF: AG-45B-0012-KI01) are unsuitable for measuring c-APRIL and therefore measure an incomplete total APRIL amount. The Inventors developed a method for quantifying the total amount of APRIL with two antibodies that specifically bind to different epitopes of denatured nc-APRIL and c-APRIL. The invented method can reliably detect and quantify total APRIL.

In certain embodiments, the invention relates to a method for quantifying the amount of c-APRIL in a sample, the method comprising the steps of: a) quantifying the amount of nc-APRIL in a first portion of the sample with the method according to embodiment 17; b) quantifying the total amount of nc-APRIL and c-APRIL in a second portion of the sample with the method according to embodiment 18, wherein the second portion of the sample has been denatured; and c) quantifying the concentration of c-APRIL in the sample, wherein quantifying the concentration of c-APRIL in the sample involves subtracting the concentration of nc-APRIL in the sample from the total concentration of nc-APRIL and c-APRIL in the sample.

Accordingly, in certain embodiments, the method of the invention can reliably detect and quantify c-APRIL. In a further embodiment of the invention measurements of nc-APRIL, of c-APRIL and/or of total APRIL can be used to calculate ratios thereof.

In certain embodiments, the invention relates to a sandwich enzyme-linked immunosorbent assay (ELISA) method.

In certain embodiments, the invention relates to an ELISA method, wherein the second monoclonal antibody is conjugated to a detection moiety or a binding moiety (e.g., biotin). In other embodiments of the invention, the detection moiety or binding moiety is conjugated to a third antibody that binds to the complex comprising the antigen and second monoclonal antibody.

In some embodiments, the invention relates to an antibody that comprises a variable heavy (VH) chain sequence comprising the amino acid sequence of SEQ ID NO:56 or a sequence having 90%, preferably 95% sequence identity to SEQ ID NO:56; and a variable light (VL) chain sequence comprising the amino acid sequence of SEQ ID NO:60 or a sequence having 90%, preferably 95% sequence identity to SEQ ID NO:60

In some embodiments, the invention relates to an antibody that comprises a variable heavy (VH) chain comprising CDR1 as defined in SEQ ID NO:57, CDR2 as defined in SEQ ID NO:58 and CDR3 as defined in SEQ ID NO:59 and a variable light (VL) chain comprising CDR1 as defined in SEQ ID NO:61, CDR2 as defined by the amino acid sequence: KAS and CDR3 as defined in SEQ ID NO:62.

In some embodiments, the invention relates to an antibody that comprises a variable heavy (VH) chain sequence comprising the amino acid sequence of SEQ ID NO:42, or a sequence having 90%, preferably 95% sequence identity to SEQ ID NO:42; and a variable light (VL) chain sequence comprising the amino acid sequence of SEQ ID NO:46, or a sequence having 90%, preferably 95% sequence identity to SEQ ID NO:46.

In some embodiments, the invention relates to an antibody that comprises a variable heavy (VH) chain comprising CDR1 as defined in SEQ ID NO:43, CDR2 as defined in SEQ ID NO:44 and CDR3 as defined in SEQ ID NO:45 and a variable light (VL) chain comprising CDR1 as defined in SEQ ID NO:47, CDR2 as defined by the amino acid sequence: GAS and CDR3 as defined in SEQ ID NO:48.

In some embodiments, the invention relates to an antibody that comprises a variable heavy (VH) chain sequence comprising the amino acid sequence of SEQ ID NO:49, or a sequence having 90%, preferably 95% sequence identity to SEQ ID NO:49; and a variable light (VL) chain sequence comprising the amino acid sequence of SEQ ID NO:53, or a sequence having 90%, preferably 95% sequence identity to SEQ ID NO:53.

In some embodiments, the invention relates to an antibody that comprises a variable heavy (VH) chain comprising CDR1 as defined in SEQ ID NO:50, CDR2 as defined in SEQ ID NO:51 and CDR3 as defined in SEQ ID NO:52 and a variable light (VL) chain comprising CDR1 as defined in SEQ ID NO:54, CDR2 as defined by the amino acid sequence: LVS and CDR3 as defined in SEQ ID NO:55.

In certain embodiments, the invention relates to a method for quantifying the amount of nc-APRIL, c-APRIL and/or total APRIL in a sample wherein the first monoclonal antibody comprises a variable heavy (VH) chain sequence comprising the amino acid sequence of SEQ ID NO:56 or a sequence having 90%, preferably 95% sequence identity to SEQ ID NO:56; and a variable light (VL) chain sequence comprising the amino acid sequence of SEQ ID NO:60 or a sequence having 90%, preferably 95% sequence identity to SEQ ID NO:60; and wherein the second monoclonal antibody comprises a variable heavy (VH) chain sequence comprising the amino acid sequence of SEQ ID NO:42, or a sequence having 90%, preferably 95% sequence identity to SEQ ID NO:42; and a variable light (VL) chain sequence comprising the amino acid sequence of SEQ ID NO:46, or a sequence having 90%, preferably 95% sequence identity to SEQ ID NO:46.

In certain embodiments, the invention relates to a method for quantifying the amount of nc-APRIL, c-APRIL and/or total APRIL in a sample wherein the first monoclonal antibody comprises a variable heavy (VH) chain sequence comprising the amino acid sequence of SEQ ID NO:56 or a sequence having 90%, preferably 95% sequence identity to SEQ ID NO:56; and a variable light (VL) chain sequence comprising the amino acid sequence of SEQ ID NO:60 or a sequence having 90%, preferably 95% sequence identity to SEQ ID NO:60; and wherein the second monoclonal antibody comprises a variable heavy (VH) chain sequence comprising the amino acid sequence of SEQ ID NO:49, or a sequence having 90%, preferably 95% sequence identity to SEQ ID NO:49; and a variable light (VL) chain sequence comprising the amino acid sequence of SEQ ID NO:53, or a sequence having 90%, preferably 95% sequence identity to SEQ ID NO:53.

In certain embodiments of the invention, an antibody of the invention, or antigen-binding fragment thereof, specifically binding to non-canonical APRIL (nc-APRIL), comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:42, SEQ ID NO:49, or SEQ ID NO:56. In certain embodiments, a VH sequence having at least 90%, 91%, 92%, 93%, 94%, 95%, 96%, 97%, 98%, or 99% identity to the amino acid sequence of SEQ ID NO:42, SEQ ID NO:49 or SEQ ID NO:56 contains substitutions (e.g., conservative substitutions), insertions, or deletions relative to the reference sequence, but an antibody of the invention, or antigen-binding fragment thereof, specifically binding to non-canonical APRIL (nc-APRIL), comprising that sequence retains the ability to specifically bind to non-canonical APRIL (nc-APRIL). In certain embodiments, a total of 1 to 10 amino acids have been substituted, inserted and/or deleted in SEQ ID NO:42, SEQ ID NO:49, or SEQ ID NO:56. In certain embodiments, a total of 1 to 5 amino acids have been substituted, inserted and/or deleted in SEQ ID NO:42, SEQ ID NO:49, or SEQ ID NO:56. In certain embodiments, substitutions, insertions, or deletions occur in regions outside the CDRs (i.e., in the FRs). In a preferred embodiment of the invention, a total of 10 amino acids in SEQ ID NO:42, SEQ ID NO:49, or SEQ ID NO:56 have been substituted to optimize the expression in mammalian cells. Optionally, an antibody of the invention, or antigen-binding fragment thereof, specifically binding to nc-APRIL, comprises the VH sequence of SEQ ID NO:42, SEQ ID NO:49 or SEQ ID NO:56, including post-translational modifications of that sequence.

In another aspect, an antibody of the invention, or antigen-binding fragment thereof, specifically binding to nc-APRIL, is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:46, SEQ ID NO:53 or SEQ ID NO:60. In certain embodiments of the invention, a VL sequence having at least 90%, 91%, 92%, 93%, 94%, 95%, 96%, 97%, 98%, or 99% identity to the amino acid sequence of SEQ ID NO:46, SEQ ID NO:53 or SEQ ID NO:60 contains substitutions (e.g., conservative substitutions), insertions, or deletions relative to the reference sequence, but an antibody of the invention, or antigen-binding fragment thereof, specifically binding to nc-APRIL, comprising that sequence retains the ability to specifically bind to nc-APRIL. In certain embodiments of the invention, a total of 1 to 10 amino acids have been substituted, inserted and/or deleted in SEQ ID NO:46, SEQ ID NO:53 or SEQ ID NO:60. In certain embodiments of the invention, a total of 1 to 5 amino acids have been substituted, inserted and/or deleted in SEQ ID NO:46, SEQ ID NO:53 or SEQ ID NO:60. In certain embodiments of the invention, the substitutions, insertions, or deletions occur in regions outside the CDRs (i.e., in the FRs). In a preferred embodiment of the invention, a total of 10 amino acids in SEQ ID NO:46, SEQ ID NO:53 or SEQ ID NO:60 have been substituted to optimize the expression in mammalian cells. Optionally, the antibody of the invention, or antigen-binding fragment thereof, specifically binding to nc-APRIL, comprises the VL sequence of SEQ ID NO:46, SEQ ID NO:53 or SEQ ID NO:60, including post-translational modifications of that sequence.

In another aspect, an antibody, or antigen-binding fragment thereof, specifically binding to nc-APRIL, is provided, wherein the antibody comprises a VH as in any of the embodiments provided above, and a VL as in any of the embodiments provided above. In a some embodiments of the invention, the antibody of the invention comprises the VH and VL sequences in SEQ ID NO:42 and SEQ ID NO:46 or SEQ ID NO:49 and SEQ ID NO:53 or SEQ ID NO:56 and SEQ ID NO:60, respectively, including post-translational modifications of those sequences. In some embodiments of the invention, an antibody of the invention, or antigen-binding fragment thereof, specifically binding to nc-APRIL, comprises a humanized form of an antibody comprising the VH and VL sequences in SEQ ID NO:42 and SEQ ID NO:46 or SEQ ID NO:49 and SEQ ID NO:53 or SEQ ID NO:56 and SEQ ID NO:60, respectively. In certain embodiments of the invention, the antibody of the invention comprises the VH and VL sequences in SEQ ID NO:42 and SEQ ID NO:46 or SEQ ID NO:49 and SEQ ID NO:53 or SEQ ID NO:56 and SEQ ID NO:60, respectively, including post-translational modifications of those sequences.

In certain embodiments, the invention relates to a method for quantifying the amount of nc-APRIL, c-APRIL and/or total APRIL in a sample wherein the first monoclonal antibody comprises (a) a variable heavy (VH) chain sequence comprising the amino acid sequence of SEQ ID NO:42, or a sequence having 90%, preferably 95% sequence identity to SEQ ID NO:42; and a variable light (VL) chain sequence comprising the amino acid sequence of SEQ ID NO:46, or a sequence having 90%, preferably 95% sequence identity to SEQ ID NO:46; or (b) a variable heavy (VH) chain sequence comprising the amino acid sequence of SEQ ID NO:49, or a sequence having 90%, preferably 95% sequence identity to SEQ ID NO:49; and a variable light (VL) chain sequence comprising the amino acid sequence of SEQ ID NO:53, or a sequence having 90%, preferably 95% sequence identity to SEQ ID NO:53; and wherein the second monoclonal antibody comprises a variable heavy (VH) chain sequence comprising the amino acid sequence of SEQ ID NO:56 or a sequence having 90%, preferably 95% sequence identity to SEQ ID NO:56; and a variable light (VL) chain sequence comprising the amino acid sequence of SEQ ID NO:60 or a sequence having 90%, preferably 95% sequence identity to SEQ ID NO:60.

In certain embodiments, the invention relates to a kit for determining the level of nc-APRIL in a sample, the kit comprising a first and a second monoclonal antibody, wherein both monoclonal antibodies bind to different epitopes of nc-APRIL.

The present invention also relates to kits, in particular research kits. In order to carry out the method of the invention, the kit can be prepared by collecting necessary reagents. In certain embodiments of the invention, the kit may comprise at least one washing liquid (e.g., wash buffer), standard, liquid for sample dilution (e.g., sample diluent buffer), liquids that enable or support detection (e.g. HRP-Streptavidin concentrate, TMB substrate reagent, stop solution), liquids that support sample preparation (e.g., cell lysis buffer) and/or the like aforementioned reagents, a reaction container and specifications. By the use of such a kit, it becomes possible to carry out determination of the level of nc-APRIL in a sample by merely adding a sample and some or all of the aforementioned reagents in a particular order.

The kits of the invention may be used in the diagnosis of medical conditions like diseases. Said medical conditions, like diseases, may be any condition/disease involving altered nc-APRIL levels, such as adverse events secondary to disease-related cardiovascular process. In certain embodiments of the invention, a kit may comprise reagents that allow determination of the level of nc-APRIL and at least one additional target in a sample. Such a kit may be particularly useful by the simultaneous detection of several medical conditions.

In a particularly preferred embodiment of the present invention, the kits (to be prepared in context) of this invention or the methods and uses of the invention may further comprise or be provided with (an) instruction manual(s). For example, said instruction manual(s) may guide the skilled person (how) to employ the kit of the invention in the diagnostic uses provided herein and in accordance with the present invention. Particularly, said instruction manual(s) may comprise guidance to use or apply the herein provided methods or uses.

In certain embodiments, the invention relates to a kit using the method above (e.g., embodiment 19) for determining the level of c-APRIL in a sample. The kit may facilitate splitting and/or processing (e.g., denaturation) of the sample upon addition to the kit. The calculation of the concentration of nc-APRIL in the sample from the total concentration of nc-APRIL and c-APRIL in the sample may be facilitated by a computer-implemented program.

In certain embodiments, the invention relates to a kit using the method above (e.g., embodiment 18) for determining the level of total-APRIL in a sample. The kit may facilitate processing (e.g., denaturation) of the sample upon addition to the kit.

In certain embodiments, the invention relates to a nephelometric assay for quantifying the concentration of non-canonical APRIL (nc-APRIL) in a sample, the assay comprising the steps of: a) contacting a sample comprising nc-APRIL with at least one antibody, or antibody coupled to microparticles or microbeads specifically binding to nc-APRIL; b) transmitting light to the mixture of step (a); c) measuring a change in light scattering intensity of the mixture in response to the irradiation in step (b); and d) quantifying the concentration of nc-APRIL in the sample according to the measurement in step (c).

By determining the emission spectrum employing a light source and observing the emission intensity at a particular wavelength or particular band of wavelength, one can relate this result to known standards. For monitoring of protein-antibody complex formation, a wavelength of less than about 360 nm is preferred, partly because it will enable detection of the early stages of complex formation more quickly.

In certain embodiments, the invention relates to a nephelometric assay that measures both scattered and transmitted light, and the scattered light is typically detected at a 900 angle from the incident beam. However, different detection angles may be used.

The concentration of nc-APRIL, which may be determined in accordance with the nephelometric assay of the present invention, depends in large part upon the specific fluorometer employed and the specific reagent system utilized.

Microspheres which scatter light best have a diameter typically in the range of 380-770 nm when using visible light, typically smaller microspheres (<<100 nm) when using UV light and typically about 0.5 μm microspheres when using infrared light.

In certain embodiments of the nephelometric assay of the invention, step a) results in a complex with an increased diameter compared to its ingredients (e.g., nc-APRIL and antibody coupled to microparticles). In embodiments using microparticles or microbeads, the size of the microbead is chosen such that the diameter difference upon step a) can be detected by the difference in light scattering. For example, microparticles or microbeads with a diameter of less than 0.1 μm (poor scatterers) are used, which grow to a size where they scatter light much better upon contacting in step a). Therefore, in a preferred embodiment microparticles or microbeads with a diameter 0.1 μm are measured using 340 nm light.

In certain embodiments, the invention relates to a nephelometric assay that uses a direct agglutination format. In an example of such a direct agglutination format, at least one antibody of the invention is coupled to microparticles or microbeads specifically binding to nc-APRIL and form a detectable complex upon contacting the nc-APRIL in the sample.

In certain embodiments, the invention relates to a nephelometric assay that uses a competitive inhibition of agglutination format. In an example of such a competitive inhibition of agglutination format, at least one initially uncoupled antibody of the invention specifically binding to nc-APRIL forms a detectable complex with at least one microbead- or microparticle-coupled antigen (e.g. nc-APRIL-coated microbeads) and the detectable complex formation is inhibited by nc-APRIL in the sample.

In certain embodiments, the invention relates to a nephelometric assay that uses a dual particle assay format. In an example of such a dual particle assay format, at least one antibody of the invention that is coupled to microparticles or microbeads specifically binding to nc-APRIL forms a detectable complex with at least one microbead- or microparticle-coupled antigen and the detectable complex formation is inhibited by nc-APRIL in the sample.

In certain embodiments of the invention, the antibody of the invention used in the nephelometric assay described herein is an antigen-binding fragment, preferably an F(ab′)2 fragment. The antigen-binding fragment in the nephelometric assay of the invention can be initially uncoupled or bound to microparticles or microbeads.

Other parameters (e.g., buffers and ionic species, optimal pH, solubility enhancers, temperature) may be selected as previously described (e.g., in EP 0,155,330; U.S. Pat. No. 4,401,387; Thompson, John C., et al. “Kinetics and proposed mechanism of the reaction of an immunoinhibition, particle-enhanced immunoassay.” Clinical chemistry 43.12 (1997): 2384-2389; Sun, Qiqi, et al. “A Low-Cost Micro-Volume Nephelometric System for Quantitative Immunoagglutination Assays.” Sensors 19.20 (2019): 4359; ABOVETHEREST, BEADS. “TechNote 304 Light-Scattering Assays.”) In certain embodiments, the invention relates to a nephelometric assay as described above, wherein the steps (a) to (c) are repeated with at least one dilution of the sample comprising nc-APRIL and/or the at least one antibody.

By the at least one dilution of the sample comprising nc-APRIL reference values (e.g. a standard curve) can be generated.

In certain embodiments, the invention relates to the nephelometric assay for quantifying the concentration of non-canonical APRIL (nc-APRIL) in a sample, as described herein, wherein the at least one antibody specifically binding to nc-APRIL comprises (a) comprises a variable heavy (VH) chain comprising CDR3 as defined in SEQ ID NO:45 and a variable light (VL) chain comprising CDR3 as defined in SEQ ID NO:48; or (b) comprises a variable heavy (VH) chain comprising CDR3 as defined in SEQ ID NO:52 and a variable light (VL) chain comprising CDR3 as defined in SEQ ID NO:55; or (c) comprises a variable heavy (VH) chain comprising CDR3 as defined in SEQ ID NO:59 and a variable light (VL) chain comprising CDR3 as defined in SEQ ID NO:62.

In certain embodiments, the invention relates to the nephelometric assay for quantifying the concentration of non-canonical APRIL (nc-APRIL) in a sample, as described herein, wherein the at least one antibody specifically binding to nc-APRIL comprises (a) comprises a variable heavy (VH) chain comprising CDR1 as defined in SEQ ID NO:43, CDR2 as defined in SEQ ID NO:44 and CDR3 as defined in SEQ ID NO:45 and a variable light (VL) chain comprising CDR1 as defined in SEQ ID NO:47, CDR2 as defined by the amino acid sequence GAS and CDR3 as defined in SEQ ID NO:48; or (b) comprises a variable heavy (VH) chain comprising CDR1 as defined in SEQ ID NO:50, CDR2 as defined in SEQ ID NO:51 and CDR3 as defined in SEQ ID NO:52 and a variable light (VL) chain comprising CDR1 as defined in SEQ ID NO:54, CDR2 as defined by the amino acid sequence: LVS and CDR3 as defined in SEQ ID NO:55; or (c) comprises a variable heavy (VH) chain comprising CDR1 as defined in SEQ ID NO:57, CDR2 as defined in SEQ ID NO:58 and CDR3 as defined in SEQ ID NO:59 and a variable light (VL) chain comprising CDR1 as defined in SEQ ID NO:61, CDR2 as defined by the amino acid sequence: KAS and CDR3 as defined in SEQ ID NO:62.

In certain embodiments, the invention relates to the nephelometric assay for quantifying the concentration of non-canonical APRIL (nc-APRIL) in a sample, as described herein, wherein the at least one antibody specifically binding to nc-APRIL comprises a variable heavy (VH) chain sequence comprising the amino acid sequence of SEQ ID NO:42, or a sequence having 90%, preferably 95% sequence identity to SEQ ID NO:42; and a variable light (VL) chain sequence comprising the amino acid sequence of SEQ ID NO:46, or a sequence having 90%, preferably 95% sequence identity to SEQ ID NO:46.

In certain embodiments, the invention relates to the nephelometric assay for quantifying the concentration of non-canonical APRIL (nc-APRIL) in a sample, as described herein, wherein the at least one antibody specifically binding to nc-APRIL comprises a variable heavy (VH) chain sequence comprising the amino acid sequence of SEQ ID NO:49, or a sequence having 90%, preferably 95% sequence identity to SEQ ID NO:49; and a variable light (VL) chain sequence comprising the amino acid sequence of SEQ ID NO:53, or a sequence having 90%, preferably 95% sequence identity to SEQ ID NO:53; or (c) a variable heavy (VH) chain sequence comprising the amino acid sequence of SEQ ID NO:56 or a sequence having 90%, preferably 95% sequence identity to SEQ ID NO:56; and a variable light (VL) chain sequence comprising the amino acid sequence of SEQ ID NO:60 or a sequence having 90%, preferably 95% sequence identity to SEQ ID NO:60.

In certain embodiments, the invention relates to a method for predicting and/or diagnosing adverse events secondary to disease-related cardiovascular process, such as, hypertriglyceridemia, metabolic syndrome, non-alcoholic steatohepatitis, diabetes mellitus type 2, atherogenic dyslipidemia, cardiovascular events and/or atherosclerosis in a subject, the method comprising the steps of: (a) determining the concentration of non-canonical APRIL (nc-APRIL) in a sample that has been obtained from said subject; (b) comparing the concentration of nc-APRIL that has been determined in step (a) to a reference value; and

(c) predicting and/or diagnosing hypertriglyceridemia, metabolic syndrome, non-alcoholic steatohepatitis and diabetes mellitus type 2, atherogenic dyslipidemia, cardiovascular events and/or atherosclerosis in said subject based on the comparison made in step (b).

In certain embodiments, the invention relates to a method for predicting mortality risk in subjects suffering from adverse events secondary to disease-related cardiovascular process, such as, hypertriglyceridemia, metabolic syndrome, non-alcoholic steatohepatitis, diabetes mellitus type 2, atherogenic dyslipidemia, cardiovascular events and/or atherosclerosis, the method comprising the steps of: (a) determining the concentration of nc-APRIL in a sample that has been obtained from said subject; (b) comparing the concentration of nc-APRIL that has been determined in step (a) to a reference value; and (c) determining the mortality risk of said subject based on the comparison made in step (b).

In certain embodiments, the invention relates to a method for determining whether a subject is susceptible to the treatment of adverse events secondary to disease-related cardiovascular process, such as, hypertriglyceridemia, metabolic syndrome, non-alcoholic steatohepatitis and diabetes mellitus type 2, atherogenic dyslipidemia, cardiovascular events and/or atherosclerosis, the method comprising the steps of: (a) determining the concentration of nc-APRIL in two or more samples that have been obtained from said subject at an earlier and a later time point; (b) determining that said subject is susceptible to the treatment of hypertriglyceridemia, metabolic syndrome, non-alcoholic steatohepatitis, diabetes mellitus type 2, atherogenic dyslipidemia, cardiovascular events and/or atherosclerosis, if the concentration of nc-APRIL is higher in a sample that has been obtained at a later time point compared to a sample that has been obtained at an earlier time point; or determining that said subject is not susceptible to the treatment of hypertriglyceridemia, metabolic syndrome, non-alcoholic steatohepatitis and diabetes mellitus type 2, atherogenic dyslipidemia, cardiovascular events and/or atherosclerosis, if the concentration of nc-APRIL is similar or lower in a sample that has been obtained at a later time point compared to a sample that has been obtained at an earlier time point.

Traditional biomarkers have limitations e.g., regarding specificity, cost, and/or intraindividual variability (Dhingra R, Vasan R S. Trends Cardiovasc Med. 2017; 27(2):123-133).

Previous attempts to use APRIL as a biomarker, for example, for diabetes only resulted in a slight negative correlation with blood glucose, which indicated that APRIL may be unsuitable as a reliable predictor for disease (Santos, Adriana Carvalho, et al., 2015, Plos One, v. 10, n. 10, p. 1-8).

Whereas canonical APRIL serum levels in humans do not show any correlation, levels of the newly discovered nc-APRIL in serum surprisingly correlate with cardiovascular and all-cause mortality in humans (Example 12).

Accordingly, this correlation can be used to create reference values for the use as a novel, unexpected tool for predicting and/or diagnosing, determining whether a subject is susceptible to the treatment of, predicting mortality risk in subjects suffering from adverse events secondary to disease-related cardiovascular process, such as hypertriglyceridemia, metabolic syndrome, non-alcoholic steatohepatitis and diabetes mellitus type 2, atherogenic dyslipidemia, cardiovascular events and/or atherosclerosis. Further, as a novel biomarker, nc-APRIL can be useful in combination with traditional biomarkers.

In certain embodiments, the invention relates to a method for determining whether a subject is susceptible to the treatment of adverse events secondary to disease-related cardiovascular process as described above, wherein the earlier time point is before the beginning of the treatment and the later time point is after the beginning of the treatment. Therefore, in certain embodiments of the invention, initial treatment response can be determined by having a time point before (e.g. 3, 2, 1 month(s), 3, 2, 1, week(s), 6, 5, 4, 3, 2, 1 day(s) 12, 8, 6, 4, 3, 2, 1 hour(s) or immediately before) treatment begin and a certain time (e.g. after 1, 2, 3, 4, 5, 6 day(s), after 1, 2, 3 week(s), after 1, 2, 4, 6 months or after 1 year) after treatment begin. Dose, treatment regime and/or treatment continuation may be adapted based on the results of the method for determining whether a subject is susceptible to the treatment, preferably under considerations of further factors relevant for treatment. Further factors relevant for treatment may include the particular type of adverse event secondary to disease-related cardiovascular process being treated, the particular subject being treated, the clinical condition of the subject, the progression of adverse event secondary to disease-related cardiovascular process, the site of delivery of the agent(s), the method of administration, and other factors known to medical practitioners.

In certain embodiments, the invention relates to a method for determining whether a subject is susceptible to the treatment of adverse events secondary to disease-related cardiovascular process as described above, wherein the earlier and the later time points are after the beginning of the treatment. Therefore, in certain embodiments of the invention, treatment response can be monitored over time (e.g., hourly, daily, weekly, every two weeks, monthly, every 2, 4, 6 months, annually). Dose, treatment regime and/or treatment continuation may be adapted based on the results of the method for determining whether a subject is susceptible to the treatment, preferably under considerations of further factors relevant for the treatment described above.

In certain embodiments, the invention relates to a method for determining whether a subject is susceptible to the treatment of adverse events secondary to disease-related cardiovascular process as described above, with a treatment comprising the use of the antibody, or antigen-binding fragment thereof, specifically binding to APRIL as described herein, or the comprising one of the pharmaceutical composition described herein.

In certain embodiments, the invention relates to a method for predicting and/or diagnosing adverse events secondary to disease-related cardiovascular process as described above, a method for predicting mortality risk in subjects suffering from adverse events secondary to disease-related cardiovascular process as described above and/or a method for determining whether a subject is susceptible to the treatment of adverse events secondary to disease-related cardiovascular process as described above, wherein the cardiovascular events comprise myocardial infarction, stroke, peripheral artery disease, angina pectoris and/or urgent hospitalization for angina leading to revascularization.

In certain embodiments, the invention relates to a method for predicting and/or diagnosing adverse events secondary to disease-related cardiovascular process as described above, a method for predicting mortality risk in subjects suffering from adverse events secondary to disease-related cardiovascular process as described above and/or a method for determining whether a subject is susceptible to the treatment of adverse events secondary to disease-related cardiovascular process as described above, wherein the sample is or comprises human serum or human plasma.

The method of the invention is suitable to detect nc-APRIL, c-APRIL and/or total APRIL in complex matrices, such as, human serum or human plasma (FIG. 11, FIG. 12, FIG. 17). Since human blood or human serum is easily accessible and the systemic level of nc-APRIL is associated with cardiovascular and all-cause mortality (FIG. 9, Example 16, Example 17), the method for predicting and/or diagnosing adverse events secondary to disease-related cardiovascular process as described above, the method for predicting mortality risk in subjects suffering from adverse events secondary to disease-related cardiovascular process as described above and/or the method for determining whether a subject is susceptible to the treatment of adverse events secondary to disease-related cardiovascular process as described above is surprisingly useful, if the sample comprise human serum or human plasma.

In certain embodiments, the invention relates to a method for predicting and/or diagnosing adverse events secondary to disease-related cardiovascular process as described above, a method for predicting mortality risk in subjects suffering from adverse events secondary to disease-related cardiovascular process as described above and/or a method for determining whether a subject is susceptible to the treatment of adverse events secondary to disease-related cardiovascular process as described above, wherein the concentration of nc-APRIL is determined with one or more antibodies specifically binding to nc-APRIL.

In certain embodiments, the invention relates to a method for predicting and/or diagnosing adverse events secondary to disease-related cardiovascular process as described above, a method for predicting mortality risk in subjects suffering from adverse events secondary to disease-related cardiovascular process as described above, a method for determining whether a subject is susceptible to the treatment of adverse events secondary to disease-related cardiovascular process as described above, wherein the concentration of nc-APRIL is determined with the method according to the method for quantifying the concentration of nc-APRIL as described herein, the nephelometric assay for quantifying the concentration of nc-APRIL as described herein, and/or a kit for determining the level of nc-APRIL as described herein.

In certain embodiments, the invention relates to any of the methods or kits described above, wherein at least one antibody of the invention specifically binding to nc-APRIL binds to an epitope within the amino acid sequence SEQ ID NO:64 and/or at least one antibody of the invention specifically binding to nc-APRIL binds to an epitope within the amino acid sequence SEQ ID NO:96.

In certain embodiments of the invention, at least one antibody of the invention specifically binding to nc-APRIL, as described herein, binds at least in part to an epitope within the amino acid sequence SEQ ID NO:64.

The at least in part binding to an epitope within the amino acid sequence SEQ ID NO:64 enables the antibody of the invention, or the antigen-binding fragment thereof, to bind to an epitope that is characteristic for human APRIL. The species specificity is beneficial for certain applications, e.g., this species specificity allows distinguishing between human and mouse APRIL.

In certain embodiments of the invention, at least one antibody of the invention specifically binding to nc-APRIL binds at least in part to an epitope within the amino acid sequence SEQ ID NO:96.

The at least in part binding to an epitope within the amino acid sequence SEQ ID NO:96 enables the antibody of the invention or the antigen-binding fragment thereof, to bind to an epitope that is conserved in several species (e.g., in humans and mice, FIG. 14). This trans-species applicability is of particular use, e.g., developed treatments based on this epitope may be useful in several species and/or may offer particular translational value in research.

In certain embodiments of the invention, wherein the use of at least a first and a second antibody is comprised, the first monoclonal antibody specifically binds to an epitope within the amino acid sequence SEQ ID NO:96 and/or the second monoclonal antibody specifically binds to an epitope within the amino acid sequence SEQ ID NO:64.

A method or a kit using antibodies of the invention, or antigen binding fragments thereof, binding to these two epitope is surprisingly useful in detecting nc-APRIL (FIG. 12, FIG. 17).

In certain embodiments of the invention, wherein the use of at least a first and a second antibody is comprised, the first monoclonal antibody specifically binds to an epitope within the amino acid sequence SEQ ID NO:64 and/or the second monoclonal antibody specifically binds to an epitope within the amino acid sequence SEQ ID NO:96.

A method or a kit using antibodies, or antigen binding fragments thereof, binding to these two epitope is surprisingly useful in detecting nc-APRIL (FIG. 10, FIG. 12, FIG. 17, Example 12), particularly for the detection of nc-APRIL in serum or plasma (FIG. 12, FIG. 17, Example 12).

The nephelometric assay as described herein, may comprise at least one antibody specifically binding to nc-APRIL binds to an epitope within the amino acid sequence SEQ ID NO:64 and/or at least one antibody specifically binding to nc-APRIL binds to an epitope within the amino acid sequence SEQ ID NO:96.

The method for predicting and/or diagnosing as described herein, may comprise at least one antibody specifically binding to nc-APRIL binds to an epitope within the amino acid sequence SEQ ID NO:64 and/or at least one antibody specifically binding to nc-APRIL binds to an epitope within the amino acid sequence SEQ ID NO:96.

In certain embodiments of the invention, at least one antibody of the invention specifically binding to nc-APRIL binds to an epitope within the amino acid sequence SEQ ID NO:96 and at least one antibody specifically binding to nc-APRIL binds to an epitope within the amino acid sequence SEQ ID NO:64.

In certain embodiments of the invention, at least one antibody of the invention specifically binding to nc-APRIL binds to an epitope that is at least partially surface-exposed. It may be understood by a person skilled in the art that the epitopes may be comprised in the APRIL protein, but may also be comprised in a degradation product thereof or may be a chemically synthesized peptide.

In certain embodiments, the invention relates to a method for predicting and/or diagnosing one or more adverse events secondary to disease-related cardiovascular processes as described herein may determine the concentration of nc-APRIL with one or more of the methods, kits, or nephrologic assays for measuring nc-APRIL described herein.

In certain embodiments, the invention relates to a method for predicting mortality risk in subjects suffering from one or more adverse events secondary to disease-related cardiovascular processes as described herein may determine the concentration of nc-APRIL with one or more of the methods, kits, or nephrologic assays for measuring nc-APRIL described herein.

In certain embodiments, the invention relates to a method for determining whether a subject is susceptible to the treatment of one or more adverse events secondary to disease-related cardiovascular processes as described herein may determine the concentration of nc-APRIL with one or more of the methods, kits, or nephrologic assays for measuring nc-APRIL described herein.

In certain embodiments of the invention, the invention relates to an antibody, or an antigen-binding fragment thereof, specifically binding to nc-APRIL, wherein the antibody, or the antigen-binding fragment thereof, binds to an epitope within the amino acid sequence SEQ ID NO:64 or SEQ ID NO:96.

In certain embodiments of the invention, the invention relates to an antibody, or antigen-binding fragment thereof, specifically binding to nc-APRIL that binds to an epitope within the amino acid sequence SEQ ID NO:96.

In certain embodiments of the invention, the invention relates to an antibody, or antigen-binding fragment thereof, specifically binding to nc-APRIL that binds to an epitope within the amino acid sequence SEQ ID NO:64.

In certain embodiments of the invention, at least one antibody or an antigen-binding fragment thereof, specifically binding to nc-APRIL binds at least in part to an epitope within the amino acid sequence SEQ ID NO:96 or at least one antibody or an antigen-binding fragment thereof, specifically binding to nc-APRIL binds at least in part to an epitope within the amino acid sequence SEQ ID NO:64, preferably at least one antibody or an antigen-binding fragment thereof, specifically binding to nc-APRIL binds to an epitope within the amino acid sequence SEQ ID NO:96 or at least one antibody or an antigen-binding fragment thereof, specifically binding to nc-APRIL binds to an epitope within the amino acid sequence SEQ ID NO:64.

This antibody of the invention or an antigen-binding fragment thereof, may serve as a reference to screen for new antibodies, specifically binding to the same or to a distinct epitope of c-APRIL or of nc-APRIL. New antibody pairs specifically binding to distinct epitopes of c-APRIL or nc-APRIL can be discovered by the person skilled in the art, using such a screening.

In order to test whether an antibody in question and the antibody of the present invention recognize the same epitope, the following competition study may be carried out: Vero cells infected with 3 moi (multiplicity of infection) are incubated after 20 h with varying concentrations of the antibody in question as the competitor for 1 hour. In a second incubation step, the antibody of the present invention is applied in a constant concentration of 100 nM and its binding is flow-cytometrically detected using a fluorescence-labelled antibody directed against the constant domains of the antibody of the invention. Binding that conducts anti-proportional to the concentration of the antibody in question is indicative for that both antibodies recognize the same epitope. However, many other assays are known in the art which may be used.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

While aspects of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

### BRIEF DESCRIPTION OF FIGURES

FIG. 1: APRIL binds to heparan sulfate proteoglycan 2 (HSPG2).

Representative confocal microscopy photomicrographs of human coronary artery specimens with or without atherosclerosis stained with anti-CD31 to mark the endothelium, anti-APRIL (Aprily 2) and anti-HSPG2 antibodies.

FIG. 2: Human serum contains a non-canonical form of APRIL (nc-APRIL).

(a) APRIL standard from ELISA 1 (Invitrogen REF: BMS2008) was depleted with beads coupled to a recombinant APRIL receptor (TACI-Ig) or to an irrelevant control receptor (TNFR2-Ig), or with beads coupled to an anti-APRIL monoclonal antibody (Aprily 2) or to an irrelevant isotype-matched control, in the indicated combinations. APRIL was measured in unbound fractions using ELISA kits 1 (Invitrogen REF: BMS2008, right) or 2 (Adipogen, left). 40-times more sample was used in ELISA 1 than in ELISA 2. (b) Recombinant flag-APRIL in native or unfolded states was depleted with the indicated bead combinations. Unbound fractions were analyzed by anti-flag Western blot. (c) Normal human serum was depleted with the indicated bead combination, then analyzed for APRIL content with ELISA 1 (right) or 2 (left). 8-times more sample was used in ELISA 1 compared to ELISA 2. Dashed lines indicate signals obtained with ELISA incubation buffer. (d) The human macrophage cell line U937 was knocked-out by CRISPR/Cas9 for either BAFF (B cell activating factor), APRIL or both BAFF and APRIL. BAFF is a B cell survival cytokine related to APRIL that also binds to TACI and BCMA. APRIL in U937 cell supernatants was measured with the Invitrogen (REF: BMS2008) and Adipogen ELISAs. Both forms of APRIL are produced in WT, but none is detected in APRIL-ko. Total absence of BAFF in supernatants of BAFF-ko cells was characterized elsewhere.

FIG. 3: Apry 1.1 (anti-APRIL antibody)-bound APRIL displays enhanced binding to proteoglycans compared to APRIL alone.

Flow cytometry analysis of HEK 293 wild-type cells stained with Flag-ACRP-mAPRIL A88 (amino acids are counted from Met18 in human APRIL, and from Met9 in mouse APRIL) preincubated with or without Apry 1.1 or heparin. Results show that APRIL binding to HEK 293 wt cells is abolished by heparin, as expected for binding to PGs, but enhanced by Apry 1.1. Error bars of duplicates are smaller than symbols.

FIG. 4: Triglycerides levels in plasma of Ldlr−/− mice upon anti-APRIL antibody treatment.

Bar graphs show the triglycerides levels in plasma of Ldlr−/− mice that were treated with an isotype or an anti-APRIL antibody (Apry 1.1; 5 mg/kg; administered biweekly) and fed an atherogenic diet for 8 weeks (left: male mice (n=10 mice per group); right: female mice (n=7-8 mice per group), * P<0.05, ** P<0.01.

FIG. 5: Macrophage content of atherosclerotic plaques in the aortic root of Ldlr−/− mice upon anti-APRIL antibody treatment.

Dot plot shows the macrophage content (expressed as percentage of MAC3 stained area out of total lesion area) of atherosclerotic plaques in the aortic root of Ldlr−/− mice that were treated with an isotype or an anti-APRIL antibody (Apry 1.1; 5 mg/kg; administered biweekly) and fed an atherogenic diet for 8 weeks (only male mice were investigated in this experiment (n=10 mice per group)); * P<0.05.

FIG. 6: Triglycerides levels in the liver and the body weight of Ldlr−/− mice upon anti-APRIL antibody treatment.

Bar graphs show the triglycerides levels in the liver (A) and the final body weight (B) of Ldlr−/− male mice that were treated with an isotype or an anti-APRIL antibody (Apry 1.1; 5 mg/kg; administered biweekly) and fed an atherogenic diet for 8 weeks (n=10 mice per group).

FIG. 7: Potential role of proteoglycan—APRIL interactions in the regulation of blood lipid metabolism.

(A) Chylomicrons, VLDL and LPL are all known to interact with cell-bound or extracellular matrix PGs. Binding of LDL to the subendothelial space triggers atherosclerotic plaque formation. The binding of LPL to PGs or GPIHBP1 on endothelial cells is physiologic. (B) APRIL stimulates the immune function through canonical receptors but can also bind to PGs. Anti-APRIL antibodies stimulate binding of APRIL to PGs. Our working model is that canonical and/or non-canonical APRIL (c-APRIL; nc-APRIL) compete with the binding of VLDL and LPL. Released LPL decreases plasma triglycerides (to a greater extent than endothelium-bound LPL) by digesting triglyceride-rich lipoproteins in the circulation.

FIG. 8: APRIL bound to anti-APRIL antibodies displays enhanced binding to proteoglycans compared to APRIL alone.

BEK 293 wild-type cells were stained with Flag-ACRP-mAPRIL A88 (amino acids are counted from Met18 in human APRIL, and from Met9 in mouse APRIL) that was preincubated or not with different APRIL-specific antibodies or heparin and analyzed by flow cytometry. Results show that APRIL binding to HEK 293 wt cells is inhibited by heparin, as expected for binding to PGs, and that all tested anti-APRIL antibodies increase this binding.

FIG. 9: Association of plasma nc-APRIL levels with cardiovascular and/or all-cause mortality in individuals included in the (a) ICARAS prospective clinical study (Inflammation and Carotid Artery-Risk for Atherosclerosis Study) and (b) LURIC (The Ludwigshafen Risk and Cardiovascular Health study.

(A) The prognostic value of plasma APRIL levels in ICARAS is independent of age (years), sex (male/female), history of myocardial infarction (binary), history of stroke (binary), peripheral arterial disease (binary), body mass index (kg/m²), hypertension (binary), diabetes mellitus (binary), serum creatinine (mg/dL), glycohemoglobin A1 (%), levels of high-sensitivity C-reactive protein (mg/dL) triglycerides (mg/dL), total cholesterol levels (mg/dL), low density lipoprotein cholesterol levels (mg/dL), ICAM-1 (ng/ml), VCAM-1 (ng/ml) and statin treatment (binary).**** Log-rank P<0.0001.

FIG. 10: Characterization of two ELISAs based on monoclonal antibodies for the detection of non-canonical (nc)APRIL, and demonstration that c-APRIL and nc-APRIL are coded by the same gene.

FIG. 10A: The specificity of the Aprily 5/Aprily 1 and Aprily 5/Aprily 2 APRIL ELISAs were tested with three sets of samples and compared to the c-APRIL ELISA from Adipogen and the APRIL ELISA from Invitrogen (REF: BMS2008). The first set of samples consisted of cell supernatant (SN) of the human cell line U937 cells, either wild type (WT) or deficient for BAFF (clone B9), or deficient for APRIL (clones M105 and B110) or deficient for both BAFF and APRIL (clones L301 and P302). Deficient clones were generated with the CRISPR/Cas9 technology. The second set of samples consisted of a normal human serum (ps sera) pre-depleted on immobilized TNFR2-Fc, or on immobilized TACI-Fc, or on an immobilized irrelevant mAb (5E1), or on an immobilized anti-APRIL mAb Aprily 2. The third set of samples consisted of intravenous immunoglobulins (IVIGs), or IVIGs separated in fractions that passed through a Protein A affinity column (Pr A FT), or of a fraction that was retained and eluted from a Protein A affinity column (Pr A elution). IVIGs, were also separated in a fraction that was not retained on an affinity column of Aprily 2 (Aprily 2 FT), and a fraction that was retained on and eluted form an Aprily 2 affinity column (Aprily 2 elution). Levels of c-APRIL were measured in 20 μl of 100× concentrated supernatant of U937 cells grown in RPMI+2% FCS, or in 10 μl of pre-depleted normal human serum, or in 10 μl of IVIG input or fractions. Levels of nc-APRIL were measured with three different ELISAs in 200 μl of 100-fold concentrated S/N of U937 cells, or in 50 μl of pre-depleted normal human serum, or in 100 μl of IVIG input or fractions.

FIG. 10B: The APRIL standard from the commercial c-APRIL ELISA (Adipogen, Cat: AG-45B-0012-KI01) was measured in the c-APRIL Adipogen kit, and in the Aprily 5/Aprily 1 and Aprily 5/Aprily 2 ELISAs for nc-APRIL.

FIG. 11: The non-canonical form of human APRIL in normal human serum can be depleted by several monoclonal antibodies that recognize denatured APRIL.

Monoclonal antibodies EctoD1 (anti-EDA, as negative control), Aprily 1, 2, 3, 5, 6, 8, 9 and 10 (anti-APRIL that recognize APRIL by Western blot and therefore recognize denatured APRIL), Mahya-1 and 110.6 (anti-APRIL recognizing native APRIL) and recombinant TACI-Ig were coupled at 1 mg/ml to NHS-Sepharose. These beads (12 μl) were used to deplete 120 μl of normal human serum. 50 μl and 10 μl of depleted sera were measured with the Invitrogen (REF: BMS2008) and Adipogen APRIL ELISAs, respectively. A well without serum was used as a control for background signal (horizontal dotted lines). The figure indicates that Aprily 1, 2, 5, 6 and 8 can deplete the signal in the Invitrogen ELISA (REF: BMS2008), while Mayha-1, mAb 110.6 and TACI-Ig can deplete the signal in the Adipogen ELISA.

FIG. 12: Detection of the non-canonical form of APRIL in recombinant APRIL and in normal human serum with various combinations of Aprily 1, 2 and 5 monoclonal antibodies.

Aprily 1, Aprily 2 and APRIL 5 coated at 5 mg/ml in ELISA plates were used to capture recombinant Flag-tagged APRIL at the indicated concentrations, or the indicated volumes of normal human serum or fetal calf serum (FCS), in a total volume of 100 μl incubation buffer. Control wells with incubation buffer only were included. Captured APRIL were then revealed as indicated with biotinylated Aprily 1, 2, or 5 at 5 μg/ml, as indicated. Signal was revealed with horseradish peroxidase-coupled streptavidin and o-penylenediamine (OPD) substrate.

FIG. 13: Epitope mapping of various anti-APRIL antibodies by competition ELISA reveals that April 5 has an epitope distinct from those of Aprily 1 and 2.

An ELISA plate was coated with purified recombinant Flag-APRIL at 1 μg/ml, then incubated for 1 h with unlabelled Aprily 1, 2, 3, 5, 6, 9, or 10, mAb 104 or mAb EctoD1 at 5 μg/ml (unlabelled competitors). After a washing step, biotinylated anti-APRIL antibodies were added (at 100 ng/ml), followed by revelation with HRP-coupled streptavidin. Short white arrows indicate reduce signal when APRIL was preincubated with the non-biotinylated form of the same antibody. Signal from the negative control (EctoD1-biiot) was subtracted, and data were normalized to the average of signals obtained with incubation buffer or EctoD1 as competitor. Medium grey arrows indicate cross-competition between Aprily 1, 2 and 6, with Aprily 2 as a competitor giving the best inhibitions. Log black arrows indicate cross-competition between Aprily 3, 9 and 10, with Aprily 3 as a competitor giving the best inhibitions. Note that binding of biotinylated Aprily 5 was not inhibited by any of the competitors except unlabelled Aprily 5, suggesting that it recognizes a distinct epitope in APRIL.

FIG. 14: The anti-APRIL mAb Aprily 5 cross-reacts with human and mouse APRIL.

50 ng of Flag-mouse APRIL “m” or Flag-human APRIL “h” were analyzed by SDS-PAGE on a 12% acrylamide gel, followed by Western blotting with the indicated anti-APRIL antibodies at 1 μg/ml, followed by HRP-coupled anti-mouse secondary reagent, or with a biotinylated anti-Flag antibody (M2-biot) followed by HRP-coupled streptavidin. The micration of molecular weight markers (in kDa) are indicated on the left-hand side. This experiment indicates that Aprily 5 is the only antibody with significant cross-reaction to mouse APRIL among those tested.

FIG. 15A: Position in the crystal structure of APRIL of minimal epitopes recognized by Aprily 1, 2 and 5.

Crystal structure of mouse APRIL bound to human TACI drawn with the Software PyMol from pdb accession number 1XU1, showing one of three TACI molecules (grey, space filling), one APRIL monomer as cartoon representation (light grey) and the other two APRIL monomers as ribbon representation (light grey). The minimal epitope determined for Aprily 5 with the amino acid sequence SEQ ID NO:96 is shown in black and indicated with black arrows, while the minimal epitope common for Aprily 1 and Aprily 2 with the amino acid sequence SEQ ID NO:64 is shown in mid-intensity grey and indicated with the grey arrows. Note that the minimal sequence of epitope 5 contains an asparagine residue (N) that is part of a consensus N-glycosylation site. Sequences indicated are for the human protein and can differ in the mouse protein. Both sequences are at least partially surface-exposed.

FIG. 15B: Schematic representation of APRIL truncations used for epitope mapping of anti-APRIL antibodies.

Fc-hAPRIL is schematized as a black rectangle (Fc) followed by a white rectangle (APRIL amino acids 98-233) (amino acids are counted from Met18 in human APRIL, and from Met9 in mouse APRIL). Deletion mutants were all fused to the Fc portion and contained the amino acid sequence indicated on the right. Minimal epitopes were defined as sequences present in all constructs recognized by an antibody and not present in constructs not recognized by the antibody. Minimal epitopes for Aprily 5, Aprily 1 and 2, and Aprily 3 and 10 are shown at the top of the figure. The minimal epitopes for Aprily 1, 2 and 5 are also shown on the left, with the amino acid sequence, and the corresponding amino acid sequence in mouse APRIL. The arrow pointing down indicates an Asn residue in a N-glycosylation consensus site. The arrow pointing up indicates a difference in the mouse sequence of the minimal epitope of Aprily 1 and 2, which may explain the species specificity of these antibodies. (Amino acids are counted from Met18 in human APRIL, and from Met9 in mouse APRIL).

FIG. 15C-15E: Epitope mapping of anti-APRIL antibodies by Western blot.

Plasmids for Fc-hAPRIL constructs containing the indicated sequence of APRIL fused to an Fc portion of hIgG1 were transfected in 293T cells. Five days later, cells were harvested, washed, lysed by sonication in the presence of SDS plus DTT, size fractionated on 12% SDS PAGE and detected by Western blot with the indicated anti-APRIL monoclonal antibody, or an antibody recognizing the Fc portion of Fc-APRIL (anti-human Ig). Blots were revealed with appropriate secondary reagents coupled to HRP, and ECL reagents.

FIG. 15C: Results are shown for Aprily 1 (top panel) and Aprily 3 (bottom panel).

FIG. 15D: Results are shown for Aprily 2 (top panel) and Aprily 10 (bottom panel).

FIG. 15E: Results are shown for Aprily 5 (top panel) and an anti-human Ig antibody (bottom panel). (Amino acids are counted from Met18 in human APRIL, and from Met9 in mouse APRIL).

FIG. 16: Apolipoprotein E deficient (Apoe−/−) mice were treated biweekly with a mixture of either mouse anti-APRIL antibody (Apry1-1) and Ctrl-Ig (α-APRIL group), or TACI-Ig and isotype IgG2b (TACI-Ig group), or isotype IgG2b and Ctrl-Ig (Ctrl group) and fed an atherogenic diet for 8 weeks.
(a) Representative photomicrographs of H&E-stained aortic root lesions (50×) and dot plot of the average lesion size in the aortic origin expressed as m2/section.
(b) Representative photomicrographs of Sudan VI stained aortas and dot plot of the quantification of enface atherosclerotic lesion size in thoracic aorta expressed as percentage of total aortic area. *P<0.05, **P<0.01

FIG. 17: Investigation of hAPRIL Invitrogen (REF: BMS2008), Adipogen Aprily 5/Aprily 1 and Aprily 5/Aprily 2 ELISA kits sensitivity.

According to method described in Example 12. Recombinant APRIL standard from the Adipogen ELISA kit, or a fixed volume of 50 μl of normal human serum and plasma were measured in the Invitrogen (REF: BMS2008), Aprily 5/Aprily 1-biot and Aprily 5/Aprily 2-biot ELISA using TMB substrate for the readout. This substrate first generates a blue color that can be monitored at 620 nm as the reaction proceeds. After termination of the reaction with acid, the color turns yellow and is monitored at 450 nm. The top row of graphs shows coloration monitored at 620 nm for the APRIIL standard curve in the three different ELISAs after the indicated time points. The middle row of panels shows the coloration after acid addition at the indicated time point. The bottom row of graphs shows the signal obtained for normal human serum and plasma monitored at 620 nm at the indicated time points. A higher intensity of signal and a higher signal to noise ratio is apparent in the Aprily-based ELISAs.

FIG. 18: Expression of Fc-hAPRIL WT and with C-terminal truncations that are differentially recognized by c-APRIL and nc-APRIL-specific ELISAs.

(A) Western Blot analysis (anti-Fc detection) of Fc-hAPRIL WT (ps1307: aa 98-233 SEQ ID NO:81; full Fc-APRIL-ps1307: SEQ ID NO: 86 and SEQ ID NO:91) and Fc-hAPRIL with C-terminal truncations (ps4185: aa 98-232 SEQ ID NO:82; full Fc-APRIL-ps4185: SEQ ID NO: 87 and SEQ ID NO:92, ps4186: aa 98-231 SEQ ID NO:83; full Fc-APRIL-ps4186: SEQ ID NO:88 and SEQ ID NO:93, ps4187: aa 98-230 SEQ ID NO:84; full Fc-APRIL-ps4187: SEQ ID NO:89 and SEQ ID NO:94, ps4188: aa 98-229 SEQ ID NO:85; full Fc-APRIL-ps4188: SEQ ID NO:90 and SEQ ID NO:95) (amino acids are counted from Met18 in human APRIL, and from Met9 in mouse APRIL). (B) ELISA analysis of the above mentioned truncated proteins using Adipogen hAPRIL ELISA kit (Cat: AG-45B-0012-KI01) to detect c-APRIL or Aprily5/Aprily 1-biot or Aprily5/Aprily2-biot ELISA kits to detect nc-APRIL. ps: plasmid; S/N: supernatant; CE: cell extract.

Result: C-terminal truncations of Fc-APRIL are mostly insoluble. The small secreted fraction only reacts in nc-APRIL ELISA.

FIG. 19: Function of APRIL in abdominal aortic aneurysm
(A) Representative ultrasound images of the abdominal aorta of APRIL deficient and control mice before and after the induction of AAA and (B) quantification of the diameter growth on day 4 and day 13 post AAA induction

FIG. 20: Nc-APRIL and c-APRIL bind heparin.

Quantitative surface plasmon resonance (Biacore) analysis of the affinity of soluble human Fc-APRIL (total), human canonical Fc-APRIL (human Fc-c-APRIL), human non-canonical Fc-APRIL (human Fc-nc-APRIL), mouse canonical Fc-APRIL (mouse Fc-c-APRIL), mouse non-canonical Fc-APRIL (mouse Fc-nc-APRIL) and negative controls EDAR-Fc and human Fas-Fc to biotinylated heparin coupled to streptavidin Sensor Chip A (n=3 independent experiments).

FIG. 21: Native canonical and non-canonical APRIL differ in size

Flag-human APRIL (from c-APRIL ELISA 1 standards) was depleted on TACI-Fc (or TNFR2-Fc as control) and/or on Aprily2 (or mIgG1 as control). APRIL was then detected by c-APRIL-specific (A) or nc-APRIL-specific (B) ELISA. C, D, Flag-human APRIL (from APRIL ELISA 1 standards) was depleted on immobilized TACI-Fc or on Aprily2, and the flow-through was then size-fractionated by size exclusion chromatography (SEC) and detected in fractions by c-APRIL-specific (C; ELISA 1) or nc-APRIL-specific (D; ELISA 2) ELISA. TACI-Fc and Aprily2 beads used for depletion were then acid-eluted. E, F, The neutralized eluate was size-fractionated, and APRIL in fractions was detected with c-APRIL-specific (E) or nc-APRIL-specific (F) ELISA. These results indicate that Flag-c-APRIL has the size of a 3-mer, whereas nc-APRIL is much larger.

FIG. 22/23: LC-MS-based parallel reaction monitoring (PRM) analysis of tryptic digest of purified human canonical or non-canonical Fc-APRIL.

A)i), A)iii), B)i), B)iii) C)i), C)iii) canonical Fc-hAPRIL, A-C, Raw data were analysed using Skyline software and extracted product ion chromatograms (XICs) are shown either in the form of peaks (A)i), A)ii), B)i), B)ii), C)i), and C) ii)) or total sum of integrated product ion areas A)iii), A)iv), B)iii), B)iv), C)iii), and C)iv) for the three selected peptides EEQYNSTYR (SEQ ID NO: 99) (Fc part) (A), LNLSPHGTFLGFVK (tryptic C terminus APRIL) (SEQ ID NO: 100) (B) and LNLSPHGTFLGFVKL (miscleaved tryptic C terminus APRIL) (SEQ ID NO: 101) (C). MS2 fragment ion spectra for the selected peptide precursor ions are illustrated at bottom right. Although the peptide shown in A is representative for comparable injection amounts of canonical versus non-canonical Fc-APRIL, the C-terminal miscleaved full tryptic peptide shown in C is undetectable in non-canonical APRIL. Relative abundances are given in arbitrary units. Right, FASTA sequence of Fc-APRIL with selected tryptic peptide sequences underlined. Note the different scales in B (10⁹) and C (10⁶).

### DETAILED DESCRIPTION

The terms “APRIL” or “A Proliferation Inducing Ligand”, as used herein, refer to any form of APRIL. APRIL is a tumor necrosis family ligand, i.e., a TNF family ligand. The term may include APRIL from any vertebrate source, including mammals such as primates (e.g., humans and rhesus macaques) and rodents (e.g., mice and rats), unless otherwise indicated. In some embodiments the term also includes naturally occurring variants of APRIL, e.g., splice variants, truncated variants or allelic variants. In preferred embodiments APRIL refers to the secretable form of APRIL, more preferably the secretable form of human APRIL such as APRIL with the amino acid sequence as defined in SEQ ID NO:1. This secretable from of APRIL (SEQ ID NO:1) can originate from the cleavage of the maternal form of APRIL with the amino acid sequence as defined in SEQ ID NO:2. The term “APRIL” or “A Proliferation Inducing Ligand” includes but is not limited to “non-canonical APRIL” or “nc-APRIL” and “canonical APRIL” or “c-APRIL”.

The terms “nc-APRIL” or “non-canonical APRIL”, as used herein, refer to newly discovered forms of APRIL that cannot bind to cognate immune receptors. Nc-APRIL has a different spatial structure than c-APRIL. Flag-tagged c-APRIL and nc-APRIL showed markedly different sizes upon gel filtration under native conditions. (FIG. 21) and nc-APRIL binds with high affinity to heparin (but lower compared to c-APRIL) when measured by surface plasmon resonance (FIG. 20). In some embodiments, nc-APRIL is a form of APRIL that binds has a K_{D} of at least 1.1 times, at least 1.2 times, at least 1.3 times, at least 1.4 times, at least 1.5 times, at least 1.6 times, at least 1.7 times, at least 1.8 times, at least 1.9 times, at least 2 times the K_{D} of the known c-APRIL, preferably under the conditions of the experiment presented in FIG. 20. In some embodiments, nc-APRIL is a form of APRIL that binds has a K_{D} of about 1.1 to 2.5 times, about 1.2 to 2.2 times, about 1.5 to 2 times the K_{D} of the known c-APRIL, preferably under the conditions of the experiment presented in FIG. 20.

In some embodiments, the nc-APRIL refers to a form of APRIL forms a more than 3-mer multimer, preferably a multimer with a size of at least a 4-mer, at least a 5-mer, at least a 6-mer at least a 7-mer, at least a 8-mer, at least a 9-mer, at least a 10-mer, in particular under the conditions of the experiment presented in FIG. 21.

Furthermore, nc-APRIL is characterized by mass spectrometry as described herein and there is at least one form of nc-APRIL that lacks the C-terminal leucine residue compared to c-APRIL (FIG. 22, 23). In some embodiments, nc-APRIL refers to a form of APRIL, wherein the epitopes defined by the amino acid sequences SEQ ID NO:64 and SEQ ID NO:96 are accessible for antibody binding.

Nc-APRIL, wherein the epitopes defined by the amino acid sequences SEQ ID NO:64 and SEQ ID NO:96 are accessible for antibody binding can be obtained, e.g., by C-terminal truncations of 1 to 3 amino acids from the amino acid sequence as defined by SEQ ID NO: 1 (FIG. 18).

In some embodiments, the amino acid sequence of human nc-APRIL is shown in the SEQ ID NO:3, SEQ ID NO:4, SEQ ID NO:5 and/or SEQ ID NO:6. In some embodiments, the amino acid sequence of human nc-APRIL is the sequence shown in the SEQ ID NO:6. In some embodiments, the amino acid sequence of human nc-APRIL is shown in the SEQ ID NO: 5. In some embodiments, the amino acid sequence of human nc-APRIL is the sequence shown in the SEQ ID NO:4. In some embodiments, the amino acid sequence of human nc-APRIL is the sequence shown in the SEQ ID NO:3 and the spatial structure differs in the secondary, tertiary, and/or quaternary structure from the structure of c-APRIL. In some embodiments, the amino acid sequence of human nc-APRIL is the sequence shown in the SEQ ID NO:3 and the spatial structure differs in the secondary structure from the structure of c-APRIL. In some embodiments, the amino acid sequence of human nc-APRIL is the sequence shown in the SEQ ID NO:3 and the spatial structure differs in the tertiary structure from the structure of c-APRIL.

In some embodiments, the amino acid sequence of human nc-APRIL is the sequence shown in the SEQ ID NO:3 and the spatial structure differs in the quaternary structure from the structure of c-APRIL.

The terms “c-APRIL” or “canonical APRIL”, as used herein, refer to the form of APRIL than can bind to cognate immune receptors. The crystal structure of c-APRIL was previously described (Wallweber H J, et al. 2004, J Mol Biol. 343(2), 283-290; Hymowitz, Sarah G., et al., 2005, Journal of Biological Chemistry 280.8: 7218-7227). The amino acid sequence of an exemplary human c-APRIL protein is shown in SEQ ID NO:1.

The term “total APRIL”, as used herein, refers to the combined amount or concentration of nc-APRIL and canonical APRIL.

The term “antibody”, as used herein, refers to a protein of the immunoglobulin family or a polypeptide comprising fragments of an immunoglobulin that is capable of specifically binding a corresponding antigen. In general, the term “antibody” is used herein in the broadest sense and encompasses various antibody structures, including but not limited to monoclonal antibodies, polyclonal antibodies, multispecific antibodies (e.g., bispecific antibodies), mouse antibodies, chimeric antibodies, fully-human antibodies and antibody fragments so long as they exhibit the desired antigen-binding activity. Antibodies within the present invention may also be chimeric antibodies, recombinant antibodies, antigen-binding fragments of recombinant antibodies, humanized antibodies or antibodies displayed upon the surface of a phage or displayed upon the surface of a chimeric antigen receptor (CAR) T cell. Methods for producing antibodies are well known in the art (see, for example, Harlow and Lane (1988) Antibodies: A Laboratory Manual, Cold Spring Harbor Laboratory, Cold Spring Harbor, N.Y.; and U.S. Pat. No. 4,196,265).

The term “binding to” as used in the context of the present invention defines a binding (interaction) of at least two “antigen-interaction-sites” with each other. The term “antigen-interaction-site” defines, in accordance with the present invention, a motif of a polypeptide, i.e., a part of the antibody or antigen-binding fragment of the present invention, which shows the capacity of specific interaction with a specific antigen or a specific group of antigens of APRIL. Said binding/interaction is also understood to define a “specific recognition”. The term “specifically recognizing” means in accordance with this invention that the antibody is capable of specifically interacting with and/or binding to at least two amino acids of APRIL as defined herein.

The terms “specifically binding” or “specific interaction” as used in accordance with the present invention means that the antibody or antigen-binding fragment thereof of the invention does not or does not essentially cross-react with (poly) peptides of similar structures. Accordingly, the phrase “specifically binding to APRIL”, as used herein, refers to the capability of binding to APRIL with sufficient affinity and specificity such that the binding is useful as a diagnostic and/or therapeutic agent and/or analytical method in targeting APRIL.

In certain embodiments of the invention, the extent of binding of an antibody specifically binding to APRIL to an unrelated, non-APRIL protein is less than about 10% of the binding of the antibody specifically binding to APRIL as measured, e.g., by a radioimmunoassay (RIA). In certain embodiments of the invention, an antibody that binds to APRIL has a dissociation constant (Kd) of ≤1 μM, ≤100 nM, ≤10 nM, ≤5 nm, ≤4 nM, ≤3 nM, ≤2 nM, ≤1 nM, ≤0.1 nM, ≤0.01 nM, or ≤0.001 nM (e.g., 10-8 M or less, e.g. from 10-8 M to 10-13 M, e.g., from 10-9 M to 10-13 M).

Thus, under designated assay conditions, the specified antibodies and the corresponding epitope of APRIL bind to one another and do not bind in a significant amount to other components present in a sample. Specific binding to a target analyte under such conditions may require a binding moiety that is selected for its specificity for a particular target analyte. A variety of immunoassay formats may be used to select antibodies specifically reactive with a particular antigen. For example, solid-phase ELISA immunoassays are routinely used to select monoclonal antibodies specifically immunoreactive with an analyte. See Shepherd and Dean (2000), Monoclonal Antibodies: A Practical Approach, Oxford University Press and/or Howard and Bethell (2000) Basic Methods in Antibody Production and Characterization, Crc. Pr. Inc. for a description of immunoassay formats and conditions that can be used to determine specific immunoreactivity. Typically, a specific or selective reaction will be at least twice background signal to noise and more typically more than 10 to 100 times greater than the background. The person skilled in the art is in a position to provide for and generate specific binding molecules directed against the novel polypeptides. For specific binding-assays, it can be readily employed to avoid undesired cross-reactivity, for example, polyclonal antibodies can easily be purified and selected by known methods.

An antibody, specifically binding to APRIL is a molecule that binds to the APRIL antigen, such as an antibody or fragment thereof, specifically binding to APRIL at a specific recognition site or epitope as detailed further above.

The specificity of the antibody or antigen-binding fragment of the present invention may not only be expressed by the nature of the amino acid sequence of the antibody or the antigen-binding fragment as defined above but also by the epitope to which the antibody is capable of binding to. Thus, the present invention relates, in one embodiment, to an antibody or an antigen-binding fragment thereof, specifically binding to APRIL, which recognizes the same epitope as an antibody of the invention, preferably antibody Aprily 1, Aprily 2 or Aprily 5. As shown in the Examples section, the epitope is a linear epitope located within the amino acid sequences SEQ ID NO:64 or SEQ ID NO:96. In a preferred embodiment of the invention, the epitope bound by the antibodies of the invention is within the amino acid sequence SEQ ID NO:96. The amino acid positions are only indicated to demonstrate the position of the corresponding amino acid sequence in the sequence of the APRIL protein. The invention encompasses all peptides comprising the epitope defined by the amino acid sequence SEQ ID NO:64 and/or SEQ ID NO:96. In some embodiments of the invention, the peptide may be a part of a polypeptide of more than 100 amino acids in length or may be a small peptide of less than 100, preferably less than 50, more preferably less than 25 amino acids, even more preferably less than 16 amino acids. The amino acids of such peptide may be natural amino acids or non-natural amino acids (e.g., beta-amino acids, gamma-amino acids, D-amino acids) or a combination thereof. Further, the present invention may encompass the respective retro-inverso peptides of the epitopes. The peptide may be unbound or bound. It may be bound, e.g., to a small molecule (e.g., a drug or a fluorophor), to a high-molecular weight polymer (e.g., polyethylene glycol (PEG), polyethylene imine (PEI), hydroxypropylmethacrylate (HPMA), etc.) or to a protein, a fatty acid, a sugar moiety or may be inserted in a membrane.

An “antigen-binding fragment” of an antibody refers to a molecule other than an intact antibody that comprises a portion of an intact antibody and that binds the antigen to which the intact antibody binds. Examples of antibody fragments include but are not limited to Fv, Fab, Fab′, F(ab′), Fab′-SH, F(ab′)2; diabodies; linear antibodies; single-chain antibody molecules (e.g., scFv); and multispecific antibodies formed from antibody fragments.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modified “monoclonal” indicates the character of the antibody as being amongst a substantially homogeneous population of antibodies and is not to be construed as requiring production of the antibody by any particular method. As mentioned above, the monoclonal antibodies to be used in accordance with the present invention may be made by the hybridoma method described by Kohler, Nature 256 (1975), 495.

The term “Fab fragment”, as used herein, is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}2 and C_{H}3 domains of an antibody. The two heavy chain fragments are held together by two or more disulfide bonds and by hydrophobic interactions of the C_{H}3 domains.

A “F(ab′) fragment” contains one light chain and a portion of one heavy chain that contains the VH domain and the C_{H}1 domain and also the region between the C_{H}1 and C_{H}2 domains, such that an interchain disulfide bond can be formed between the two heavy chains of two Fab′ fragments to form an F(ab′)₂ molecule.

An “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two F(ab′) fragments that are held together by a disulfide bond between the two heavy chains.

An “Fv fragment” comprises the variable regions from both the heavy and light chains but lacks the constant regions.

“single-chain Fv” or “scFv” antibody fragments have, in the context of the invention, the VH and VL domains of an antibody, wherein these domains are present in a single polypeptide chain. Generally, the scFv polypeptide further comprises a polypeptide linker between the VH and VL domains, which enables the scFv to form the desired structure for antigen binding. Techniques described for the production of single chain antibodies are described, e.g., in Plückthun in The Pharmacology of Monoclonal Antibodies, Rosenburg and Moore eds. Springer-Verlag, N.Y (1994), 269-315.

Antibodies, antibody constructs, antibody fragments, antibody derivatives (all being Ig-derived) to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook (1989), loc. cit. The term “Ig-derived domain” particularly relates to (poly) peptide constructs comprising at least one CDR. Fragments or derivatives of the recited Ig-derived domains define (poly) peptides, which are parts of the above antibody molecules and/or which are modified by chemical/biochemical or molecular biological methods.

Corresponding methods are known in the art and described inter alia in laboratory manuals (see Sambrook et al., Molecular Cloning: A Laboratory Manual; Cold Spring Harbor Laboratory Press, 2nd edition (1989) and 3rd edition (2001); Gerhardt et al., Methods for General and Molecular Bacteriology ASM Press (1994); Lefkovits, Immunology Methods Manual: The Comprehensive Sourcebook of Techniques; Academic Press (1997); Golemis, Protein-Protein Interactions: A Molecular Cloning Manual Cold Spring Harbor Laboratory Press (2002)).

The term “CDR”, as used herein, refers to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in Kabat “Sequences of Proteins of Immunological Interest”, 5th edit. NIH Publication no. 91-3242 U.S. Department of Health and Human Services (1991); Chothia J. Mol. Biol. 196 (1987), 901-917 or Chothia Nature 342 (1989), 877-883.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

Accordingly, in the context of the present invention, the antibody molecule described hereinabove is selected from the group consisting of a full antibody (immunoglobulin, like an IgG1, an IgG2, an IgG2a, an IgG2b, an IgA1, an IgGA2, an IgG3, an IgG4, an IgA, an IgM, an IgD or an IgE), F(ab)-, Fab′-SH-, Fv-, F(ab′)-, Fab′-, F(ab′)2-fragment, a chimeric antibody, a CDR-grafted antibody, a humanized antibody, a fully human antibody, a bivalent antibody-construct, an antibody-fusion protein, a synthetic antibody, bivalent single chain antibody, a trivalent single chain antibody and a multivalent single chain antibody.

“Humanized” forms of non-human (e.g., murine or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Often, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Furthermore, humanized antibody may comprise residues, which are found neither in the recipient antibody nor in the imported CDR or framework sequences. These modifications are made to further refine and optimize antibody performance. In general, the humanized antibody will comprise substantially all of at least one, and typically two variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. For further details, see: JonesNature 321 (1986), 522-525; Reichmann Nature 332 (1998), 323-327 and Presta Curr Op Struct Biol 2 (1992), 593-596.

A popular method for humanization of antibodies involves CDR grafting, where a functional antigen-binding site from a non-human ‘donor’ antibody is grafted onto a human ‘acceptor’ antibody. CDR grafting methods are known in the art and described, for example, in U.S. Pat. Nos. 5,225,539, 5,693,761 and 6,407,213. Another related method is the production of humanized antibodies from transgenic animals that are genetically engineered to contain one or more humanized immunoglobulin loci, which are capable of undergoing gene rearrangement and gene conversion (see, for example, U.S. Pat. No. 7,129,084). Accordingly, in the context of the present invention, the term “antibody” relates to full immunoglobulin molecules as well as to parts of such immunoglobulin molecules (i.e., “antigen-binding fragment thereof”). Furthermore, the term relates, as discussed above, to modified and/or altered antibody molecules. The term also relates to recombinantly or synthetically generated/synthesized antibodies.

The term “polynucleotide”, as used herein, refers to a molecule such as a biopolymer composed of 13 or more nucleotide monomers bonded in a chain. Polynucleotides include but are not limited to DNA, RNA, cDNA.

“Percent (%) amino acid sequence identity” with respect to a reference polypeptide sequence refers to the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term “pharmaceutical composition”, as used herein, refers to a preparation which is in such form as to permit the biological activity of one or more active ingredients contained therein to be effective, and which contains no additional components which are unacceptably toxic to a subject to which the composition would be administered.

The term “pharmaceutically acceptable carrier”, as used herein, refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments of the invention, antibodies of the invention are used to delay development of a disease or to slow the progression of a disease.

The term “therapeutic agent”, as used herein, refers to a treatment, a treatment composition, antibodies, an antigen-binding fragment, a “pharmaceutical composition” or a combination thereof.

The terms “individual” or “subject”, as used herein, refer to an animal or a human. Animals include, but are not limited to, domesticated animals (e.g., cows, sheep, cats, dogs, and horses), primates (e.g., humans and non-human primates such as macaques), rabbits, and rodents (e.g., mice and rats). In certain embodiments of the invention, the individual or subject is a human.

The term “adverse events secondary to disease-related cardiovascular process”, as used herein, refers to any condition or event in which triglyceride levels, cholesterol levels and/or altered state of blood vessels play a role. Therefore, the term “adverse events secondary to disease-related cardiovascular process” refers in particular to, but is not limited to, hypertriglyceridemia, metabolic syndrome, non-alcoholic steatohepatitis, NAFLD, diabetes mellitus type 1, diabetes mellitus type 2, atherogenic dyslipidemia, cardiovascular events and/or atherosclerosis.

The term “hypertriglyceridemia”, as used herein, refers to or describes the physiological condition in a subject that is typically characterized by mammals having increased triglyceride levels, for example, humans having >150 mg/dl of systemic triglyceride levels. “Hypertriglyceridemia” further includes but is not limited to a physiological condition having increased triglyceride levels and being associated with overeating, obesity, diabetes mellitus and/or insulin resistance and/or metabolic syndrome, substance use (including but not limited to use of alcohol, propofol, isotretinoin, hydrochlorothiazide diuretics, beta-blockers, protease inhibitors and/or HIV medications), kidney failure, nephrotic syndrome, genetic predisposition, familial hyperlipidemia (including but not limited to type II hyperlipidemia), lipoprotein lipase deficiency, lysosomal acid lipase deficiency or cholesteryl ester storage disease, hypothyroidism, systemic lupus erythematosus, associated autoimmune responses, glycogen storage disease type 1, lipemia retinalis, hepatosplenomegaly, and/or neurological symptoms.

The term, “metabolic syndrome”, as used herein, refers to the clustering of a number of symptoms that relate to the consequences of disturbances in energy metabolism, that is the metabolism of lipids, carbohydrates and proteins. Obesity, insulin resistance, diabetes, hypertension and hyperlipidemia are the components of the syndrome. At least three of the following five criteria should be fulfilled: Blood pressure >130/85 mmHg or antihypertensive treatment, fasting plasma glucose >6.1 mmol/l, serum triglycerides >1.7 mmol/l, waist circumference >102 cm in men and >88 cm in women, HDL-cholesterol <1.0 mmol/l in men and <1.3 in women.

The term “NAFLD”, as used herein, relates a group of conditions having in common the accumulation of fat in the hepatocytes. NAFLD ranges from simple fatty liver (steatosis), to non-alcoholic steatohepatitis (NASH), to cirrhosis (irreversible, advanced scarring of the liver).

The terms “non-alcoholic steatohepatitis” or “NASH”, as used herein, collectively refer to the state where the liver develops a hepatic disorder (e.g., inflammation, ballooning, fibrosis, cirrhosis, or cancer), or the state where the liver may induce such a pathological condition, and “NASH” is distinguished from “simple steatosis”; i.e., a condition in which fat is simply accumulated in the liver, and which does not progress to another hepatic-disorder-developing condition.

The term “diabetes”, as used herein, refers to any disease characterized by a high concentration of blood glucose (hyperglycemia). Diabetes is diagnosed by demonstrating any one of the following: a fasting plasma glucose level at or above 126 mg/dL (7.0 mmol/l) or plasma glucose at or above 200 mg/dL (11.1 mmol/l) two hours after a 75 g oral glucose load as in a glucose tolerance test or symptoms of hyperglycemia and casual plasma glucose at or above 200 mg/dL (11.1 mmol/l). As used herein, diabetes includes but is not limited to “type 1 diabetes” also known as childhood-onset diabetes, juvenile diabetes, and insulin-dependent diabetes (IDDM) or “diabetes mellitus type 2”.

The term “diabetes mellitus type 2”, as used herein, refers to a form of diabetes that is characterized by hyperglycemia resulting from impaired insulin utilization coupled with the body's inability to compensate with increased insulin production. Diabetes mellitus type 2 includes but is not limited to adult-onset diabetes, obesity-related diabetes, non-insulin-dependent diabetes (NIDDM), gestational diabetes, insulin-resistant type 1 diabetes (or “double diabetes”), diabetic dyslipidemia, latent autoimmune diabetes of adults (or LADA), maturity-onset diabetes of the young (MODY).

The terms “atherogenic dyslipidemia” or “AD”, as used herein, refer to a condition characterized by elevated levels of triglycerides and small-dense low-density lipoprotein and low levels of high-density lipoprotein cholesterol as described by the EAC/ESC guidelines (Mach, François, et al. “2019 ESC/EAS Guidelines for the management of dyslipidaemias: lipid modification to reduce cardiovascular risk: The Task Force for the management of dyslipidaemias of the European Society of Cardiology (ESC) and European Atherosclerosis Society (EAS).” European heart journal41.1 (2020): 111-188). In addition, elevated levels of large TG rich very low-density lipoproteins, apolipoprotein B and oxidized low-density lipoprotein, and reduced levels of small high-density lipoproteins can be part of AD.

The term “cardiovascular event”, as used herein, refers to a failure or malfunction of any part of the circulatory system. Included in the term cardiovascular event are, inter alia, myocardial infarction, stroke, urgent hospitalization for angina leading to revascularization, peripheral artery disease, angina pectoris peripheral revascularization or amputation (in the context of peripheral artery disease).

The terms “proteoglycan” or “PG”, as used herein, refer to a class of proteins that are characterized by pronounced glycosylation. In some embodiments of the invention, the carbohydrate content of the proteoglycan is larger than the protein content, preferably the carbohydrate content is responsible for more than about 60%, more preferably more than about 70%, more preferably more than about 80%, more preferably more than about 90%, more preferably more than about 95% of the molecular weight of the PG.

“proteoglycan” or “PG” include, inter alia, “heparan sulphate”, “HS” and/or “HSPG”.

The terms “extracellular matrix”, “ECM” or “extracellular space”, as used herein, refer to a three-dimensional network of extracellular molecules, such as macromolecules, collagen, enzymes, glycoproteins, and/or molecules that provide structural and/or biochemical support to surrounding cells.

The terms “subendothelial”, “subendothelial space”, “subendothelial layer” or “subendothelial extracellular matrix”, as used herein, refer to a tissue and/or a three-dimensional adjacent to the endothelial, such as the endothelial of arteries.

The term “receptor”, as used herein, refers to chemical structures, such as proteins, that receive and/or transduce signals that may be integrated into biological systems.

The term “endogenous receptor”, as used herein, refers to a receptor naturally produced by the body of a subject, including but not limited to receptors of the tumor necrosis factor receptor superfamily.

The term “BCMA”, as used herein, refers to B-cell maturation antigen also known as tumor necrosis factor receptor superfamily member 17.

The term “CAML”, as used herein, refers to a signaling protein recognized by the TNF receptor TACI such as Calcium modulating ligand or calcium-modulating cyclophilin ligand.

The term “TACI”, as used herein, refers to “Transmembrane activator and CAML interactor” and/or tumor necrosis factor receptor superfamily member 13B.

The term “fibrates”, as used herein, refers to a class of amphipathic carboxylic acids than can be used as a therapeutic agent, including but not limiting to gemfibrozil and/or fenofibrate.

The term “statins”, as used herein, refers to HMG-CoA reductase inhibitors including but not limited to atorvastatin, fluvastatin, lovastatin, pravastatin, rosuvastatin, simvastatin and/or pitavastatin.

The term “triglyceride”, as used herein, refers to an organic molecule comprising an ester derived from glycerol and three fatty acids.

The terms, “Angiopoietin-like protein 3” or “ANGPTL3”, as used herein, refer to a protein that is encoded by the ANGPTL3 gene in humans. However, the term “Angiopoietin-like protein 3” or “ANGPTL3” can refer to any homolog, paralog or paralog of human Angiopoietin-like protein 3.

The terms “Apolipoprotein C-III” or “apoC-III”, as used herein, refer to a protein with a molecular weight of about 9 kDa and can inhibit lipoprotein lipase and hepatic lipase. In humans Apolipoprotein C-III can be encoded by the APOC3 gene. However, the term “Apolipoprotein C-III” or “apoC-III” can refer to any homolog, paralog or paralog of human Apolipoprotein C-III.

The term “auto-antibody”, as used herein, refers to an antibody produced by the body of a subject that is directed against one or more of the subject's own proteins.

The term “Glycosylphosphatidylinositol anchored high density lipoprotein binding protein 1” or “GPIHBP1”, as used herein, refers to a protein that in humans can be encoded by the GPIHBP1 gene. However, the term “Apolipoprotein C-III” or “apoC-III” can refer to any homolog, paralog or paralog of human glycosylphosphatidylinositol anchored high density lipoprotein binding protein 1.

The term “LDL” or “low-density lipoprotein”, as used herein, refers to a group of lipoproteins with a particular low density. A single LDL particle typically is about 220-275 angstroms in diameter, typically transporting about 3,000 to about 6,000 fat molecules per particle, and varying in size according to the number and mix of fat molecules contained within. The lipids carried include but are not limited to all fat molecules with cholesterol, phospholipids, and triglycerides dominant; amounts of each varying considerably. The term “LDL” or “low-density lipoprotein” can also refer to VLDL

The term “LDL-C”, as used herein, refers to the amount of cholesterol which is estimated to be contained with LDL particles. LDL-C can be directly measured or estimated using a formula, such as the Friedewald equation.

The term “VLDL” or “low-density lipoprotein”, as used herein, refers to a group of lipoproteins with a particular low density. VLDL particles typically have a diameter of 30-80 nm. The lipids by VLDL particles carried include but are not limited to all fat molecules with cholesterol, phospholipids, and triglycerides dominant; amounts of each varying considerably.

The terms “HDL” or “high-density lipoprotein”, as used herein, refer to a group of lipoproteins with a particular high density. These lipoproteins are typically composed of about 80-100 proteins per particle and transporting up to hundreds of fat molecules per particle.

The term “HDL-C”, as used herein, refers to cholesterol associated HDL. HDL-C can be directly measured or estimated using a formula.

The terms “ELISA” or “enzyme-linked immunosorbent assay”, as used herein, refer to analytical biochemistry assay. An ELISA comprises the use of antigens from the sample to which a matching antibody is applied so it can bind to the antigen. This antibody can be linked to an enzyme, and a substance containing the enzyme's substrate is added. The subsequent reaction produces a detectable signal, such as a color change. An ELISA can be used, inter alia, as a diagnostic tool in medicine, plant pathology, and biotechnology, as well as a quality control check in various industries.

The term “first monoclonal antibody”, as used herein, refers to a monoclonal antibody which binds specifically to a target protein antigen in a sample and is the first antibody used in an assay.

The term “second monoclonal antibody”, as used herein, refers to a monoclonal antibody, which is added to an assay after the addition of the first monoclonal antibody to impart detection. In certain embodiments of the invention, the second monoclonal antibody may impart detection in that it can be detected by at least one method known in the art, e.g., via a detection moiety or a binding moiety. In other embodiments of the invention, the second monoclonal antibody allows the binding of a third antibody that can be detected by at least one method known in the art, e.g., via a detection moiety or a binding moiety. The detection may be imparted by horseradish peroxidase, Alkaline phosphatase, fluorescein isothiocyanate (FITC), rhodamine, Texas Red, phycoerythrin, or biotin.

The term “nephelometric assay”, as used herein, refers to a technique that is performed by detecting the scattered light at an angle from the sample, such as blood sample, being measured.

A nephelometric assay includes, but is not limited to endpoint nephelometry and/or kinetic nephelometry.

The term “end point nephelometry”, as used herein, refers to a nephelometric technique that detects the maximum scattered light after a fixed reaction time and/or after an antigen-antibody reaction has reached equilibrium.

The term “kinetic nephelometry”, as used herein, refers to a nephelometric technique that in which the peak rate of immune-complex formation is detected.

The term “Aprily 1”, as used herein, refers to an antibody that comprises a variable heavy (VH) chain sequence comprising the amino acid sequence of SEQ ID NO:42 comprising a CDR1 as defined in SEQ ID NO:43, a CDR2 as defined in SEQ ID NO:44 and a CDR3 as defined in SEQ ID NO:45, and a variable light (VL) chain sequence comprising the amino acid sequence of SEQ ID NO:46 comprising a CDR1 as defined in SEQ ID NO:47, a CDR2 as defined by the amino acid sequence: GAS and a CDR3 as defined in SEQ ID NO:48.

The term “Aprily 2”, as used herein, refers to an antibody that comprises a variable heavy (VH) chain sequence comprising the amino acid sequence of SEQ ID NO:49 comprising a CDR1 as defined in SEQ ID NO:50, a CDR2 as defined in SEQ ID NO:51 and a CDR3 as defined in SEQ ID NO:52 and a variable light (VL) chain sequence comprising the amino acid sequence of SEQ ID NO:53 comprising a CDR1 as defined in SEQ ID NO:54, a CDR2 as defined by the amino acid sequence: LSV and a CDR3 as defined in SEQ ID NO:55.

The term “Aprily 5”, as used herein, refers to an antibody that comprises a variable heavy (VH) chain sequence comprising the amino acid sequence of SEQ ID NO:56 comprising a CDR1 as defined in SEQ ID NO:57, a CDR2 as defined in SEQ ID NO: 58 and a CDR3 as defined in SEQ ID NO: 59 and a variable light (VL) chain sequence comprising the amino acid sequence of SEQ ID NO:60 comprising a CDR1 as defined in SEQ ID NO: 61, a CDR2 as defined by the amino acid sequence: KAS and a CDR3 as defined in SEQ ID NO:62.

The term “Apry 1.1”, as used herein, refers to a single chain human anti-mouse APRIL antibody fused to the Fc portion of a mouse IgG1 that comprises variable heavy (VH) chain sequence and is commercially available from AdipoGen (Product name: anti-APRIL (mouse), mAb (rec.) (blocking) (Apry-1-1); Product code: AG-27B-0001PF).

The term “104”, as used herein, refers to an antibody that comprises a variable heavy (VH) chain sequence comprising the amino acid sequence of SEQ ID NO:7 comprising a CDR1 as defined in SEQ ID NO:8, a CDR2 as defined in SEQ ID NO:9 and a CDR3 as defined in SEQ ID NO:10 and a variable light (VL) chain sequence comprising the amino acid sequence of SEQ ID NO:11 comprising a CDR1 as defined in SEQ ID NO:12, a CDR2 as defined by the amino acid sequence: YAS and a CDR3 as defined in SEQ ID NO: 13.

The term “108”, as used herein, refers to an antibody that comprises a variable heavy (VH) chain sequence comprising the amino acid sequence of SEQ ID NO:14 comprising a CDR1 as defined in SEQ ID NO:15, a CDR2 as defined in SEQ ID NO:16 and a CDR3 as defined in SEQ ID NO:17 and a variable light (VL) chain sequence comprising the amino acid sequence of SEQ ID NO:18 comprising a CDR1 as defined in SEQ ID NO:19, a CDR2 as defined by the amino acid sequence: AAS and a CDR3 as defined in SEQ ID NO:20.

The term “110”, as used herein, refers to an antibody that comprises a variable heavy (VH) chain sequence comprising the amino acid sequence of SEQ ID NO:21 comprising a CDR1 as defined in SEQ ID NO:22, a CDR2 as defined in SEQ ID NO:23 and a CDR3 as defined in SEQ ID NO:24 and a variable light (VL) chain sequence comprising the amino acid sequence of SEQ ID NO:25 comprising a CDR1 as defined in SEQ ID NO:26, a CDR2 as defined by the amino acid sequence: GTN and a CDR3 as defined in SEQ ID NO:27.

The term “115”, as used herein, refers to an antibody that comprises a variable heavy (VH) chain sequence comprising the amino acid sequence of SEQ ID NO:28 comprising a CDR1 as defined in SEQ ID NO:29, a CDR2 as defined in SEQ ID NO:30 and a CDR3 as defined in SEQ ID NO:31 and a variable light (VL) chain sequence comprising the amino acid sequence of SEQ ID NO:32 comprising a CDR1 as defined in SEQ ID NO:33, a CDR2 as defined by the amino acid sequence GTS and a CDR3 as defined in SEQ ID NO:34.

The term “2C8”, as used herein, refers to an antibody that comprises a variable heavy (VH) chain sequence comprising the amino acid sequence of SEQ ID NO:35 comprising a CDR1 as defined in SEQ ID NO:36, a CDR2 as defined in SEQ ID NO:37 and a CDR3 as defined in SEQ ID NO:38 and a variable light (VL) chain sequence comprising the amino acid sequence of SEQ ID NO:39 comprising a CDR1 as defined in SEQ ID NO:40, a CDR2 as defined by the amino acid sequence LVS and a CDR3 as defined in SEQ ID NO41.

All publications, patent applications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document is authoritative.

Furthermore, in the claims, the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

### EXAMPLES

### Example 1

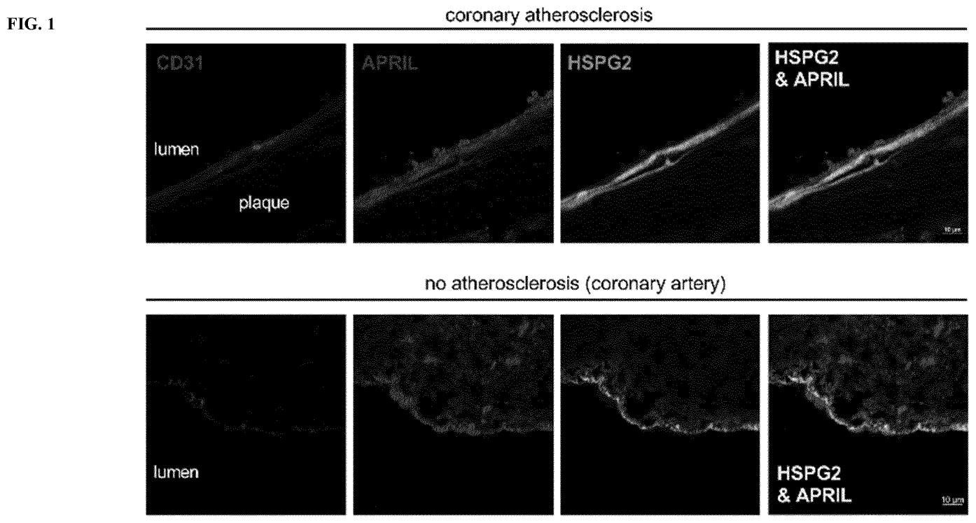

The inventors have found that APRIL is present in healthy human carotid and coronary arteries as well as in arteries with atherosclerotic plaques and directly binds to heparan sulfate proteoglycan 2 (HSPG2 or Perlecan) (FIG. 1). These data were generated by performing confocal microscopy in human arteries using the Aprily 2 antibody (which is described above) and an anti-HSPG2 specific antibody (clone A7L6; Merck).

### Example 2

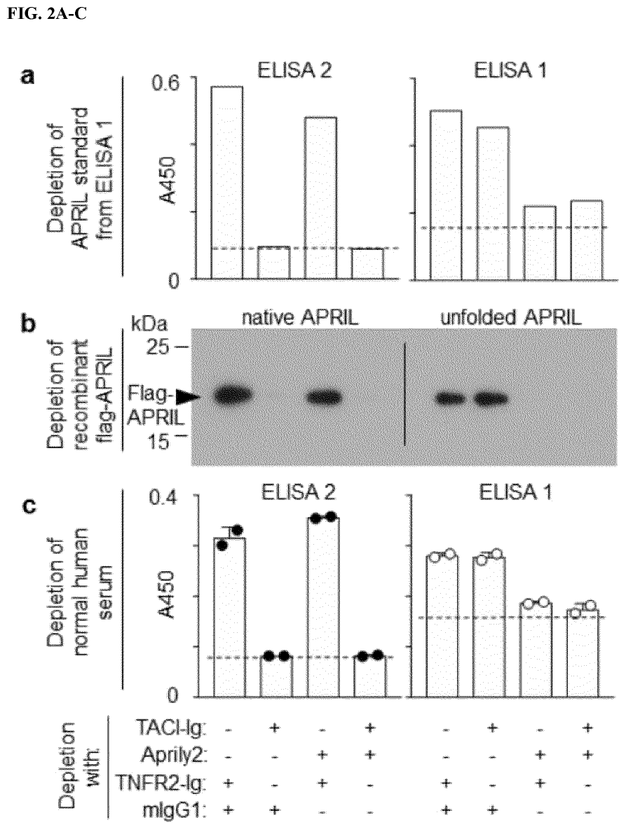

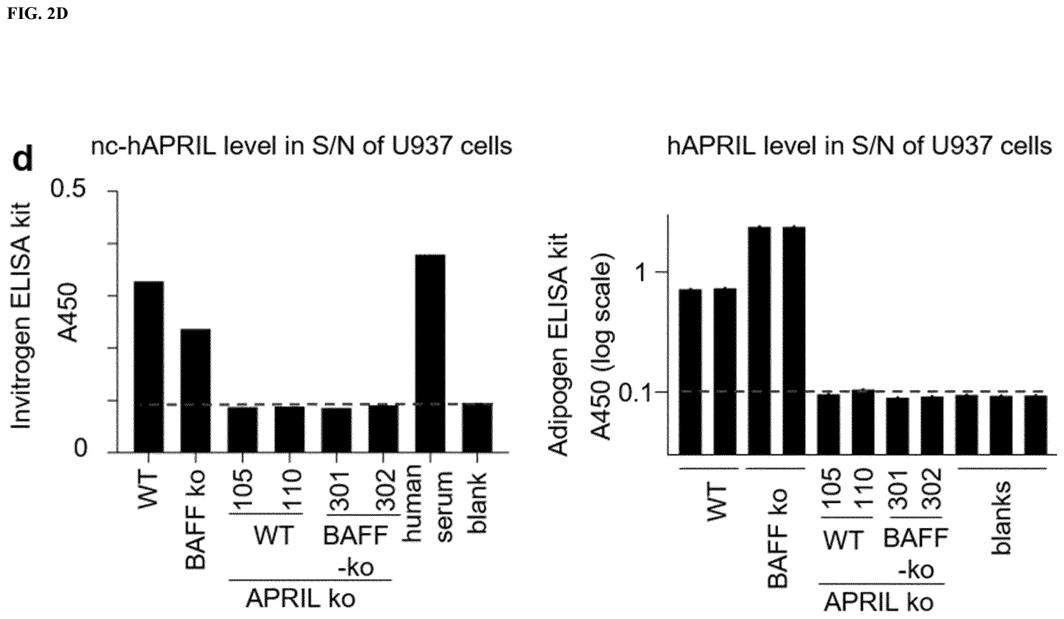

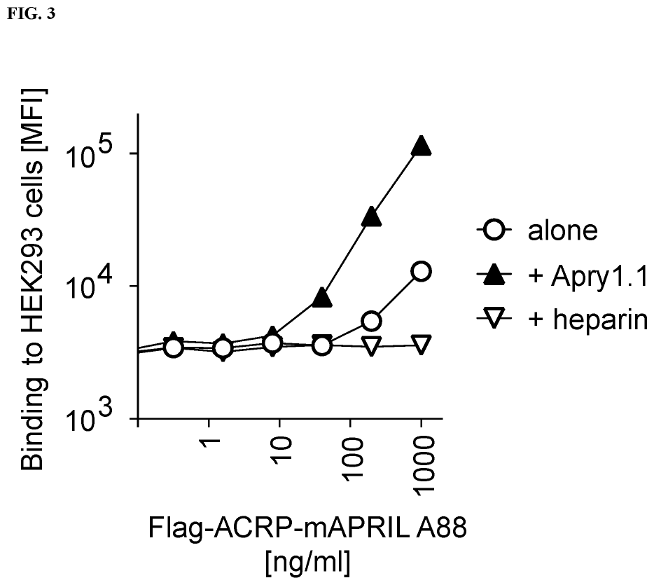

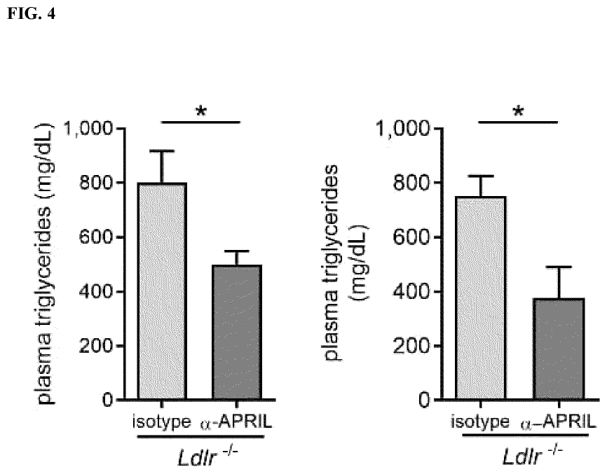

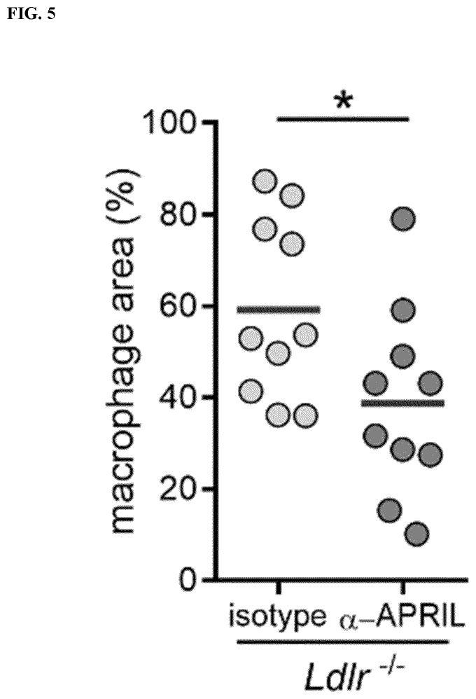

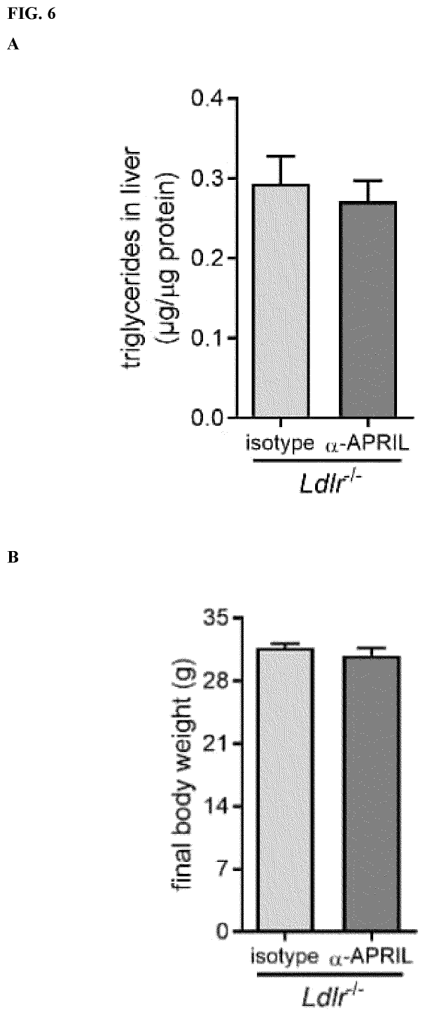

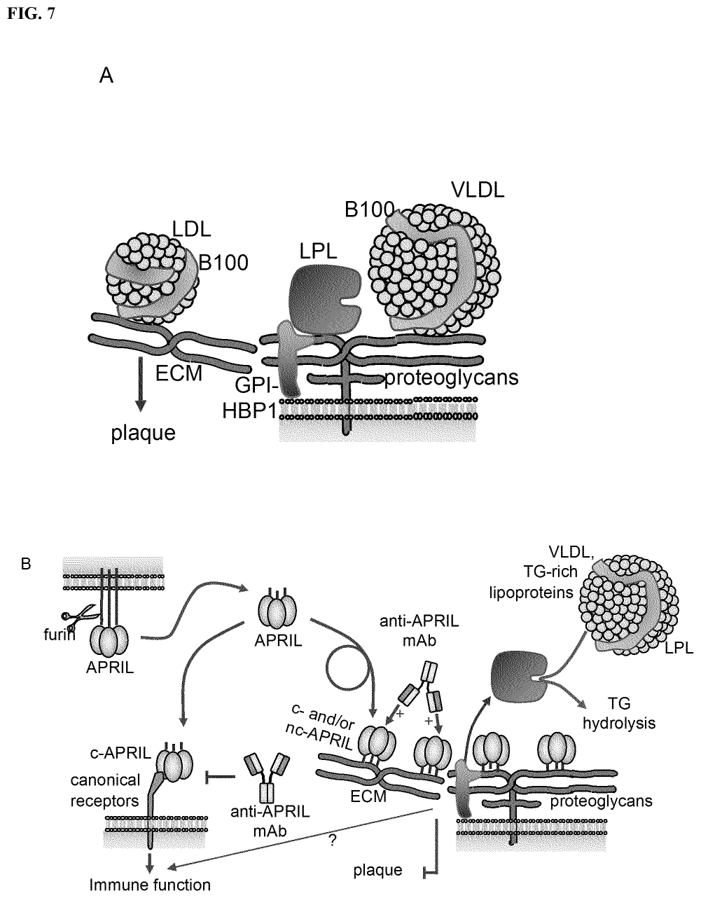

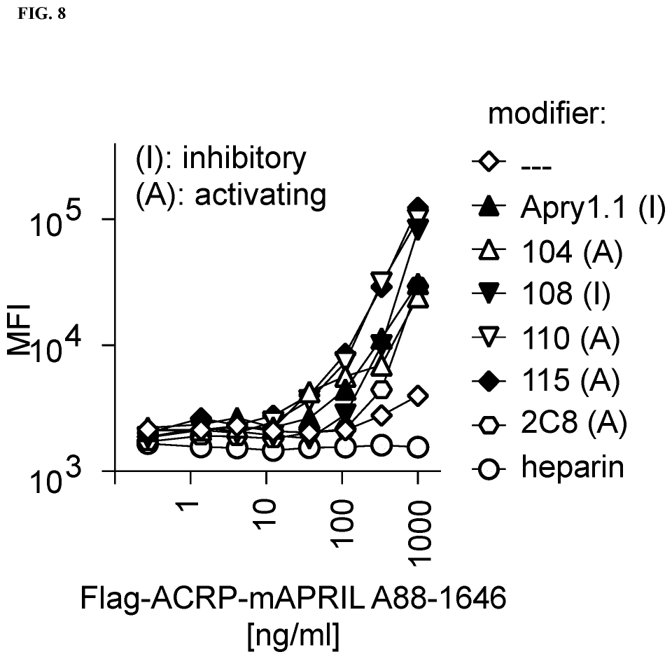

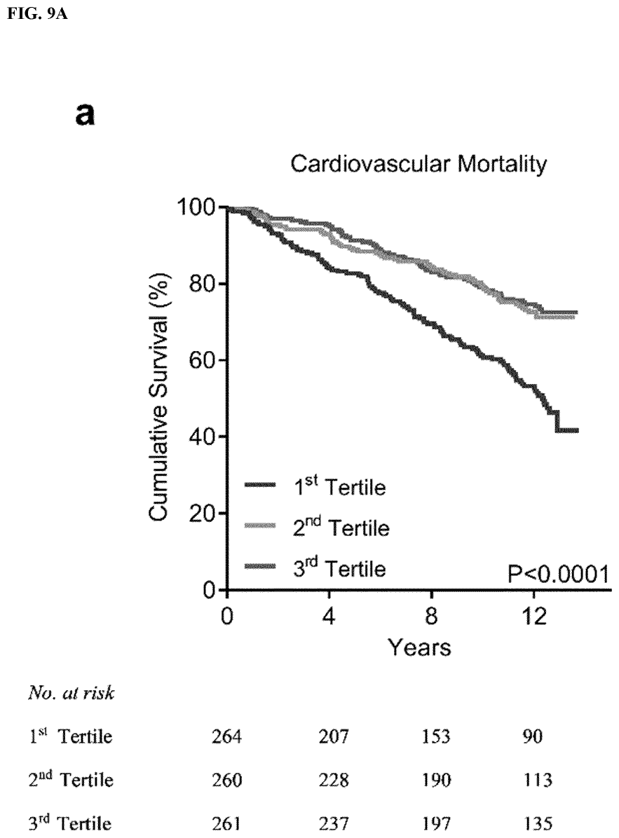

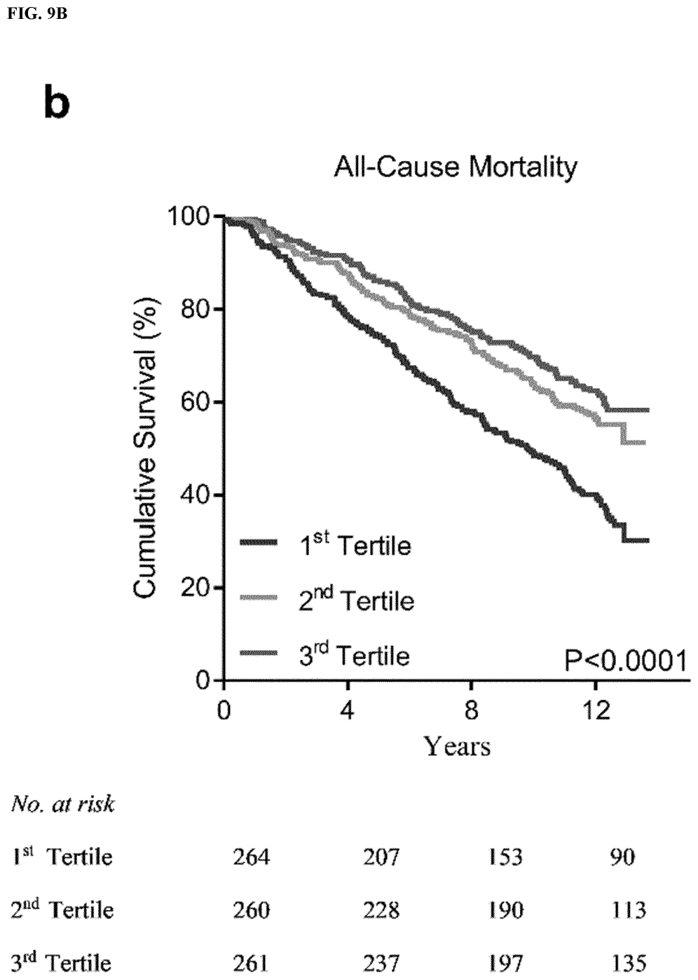

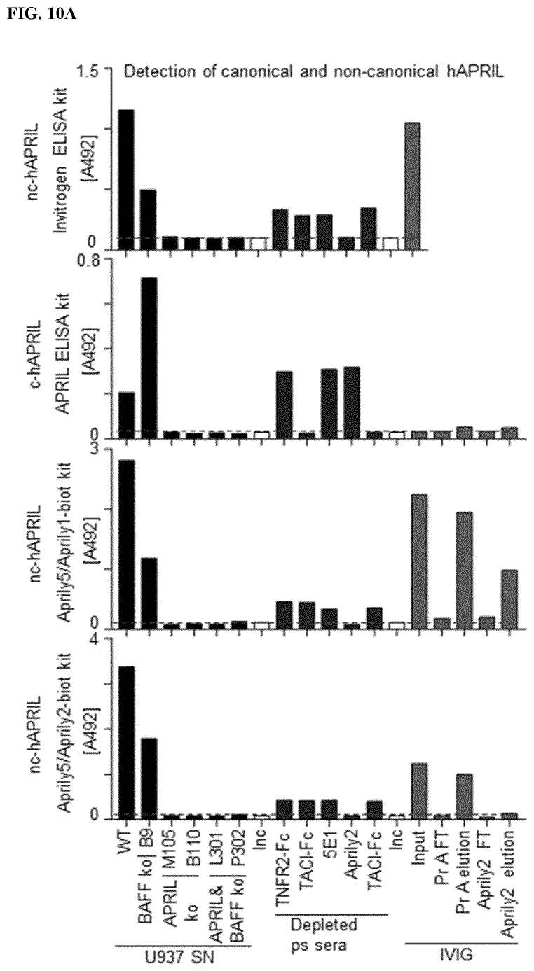

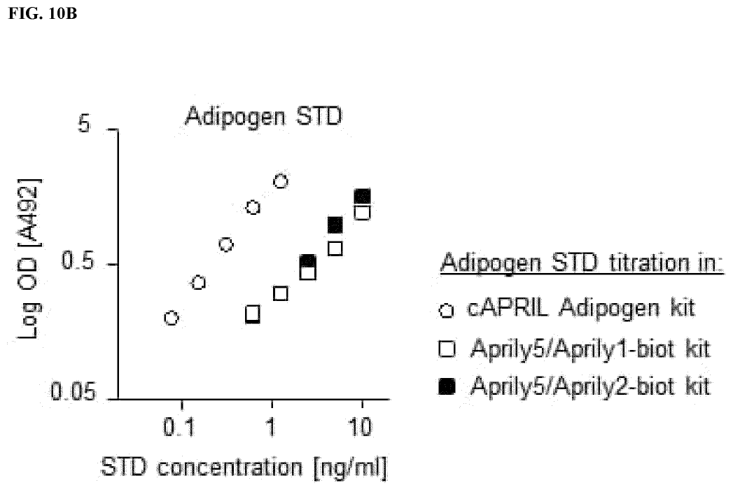

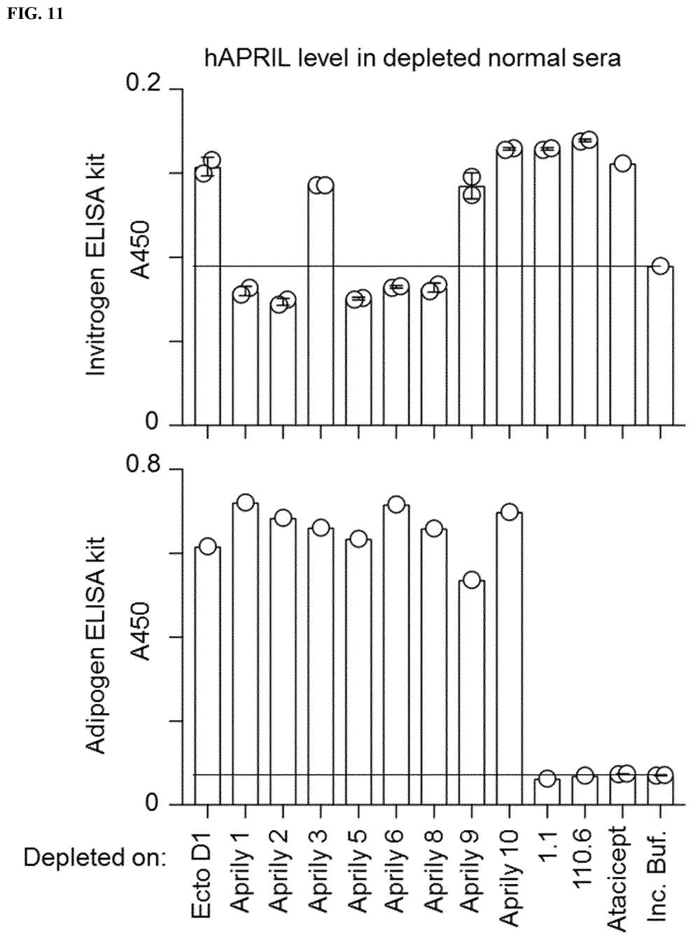

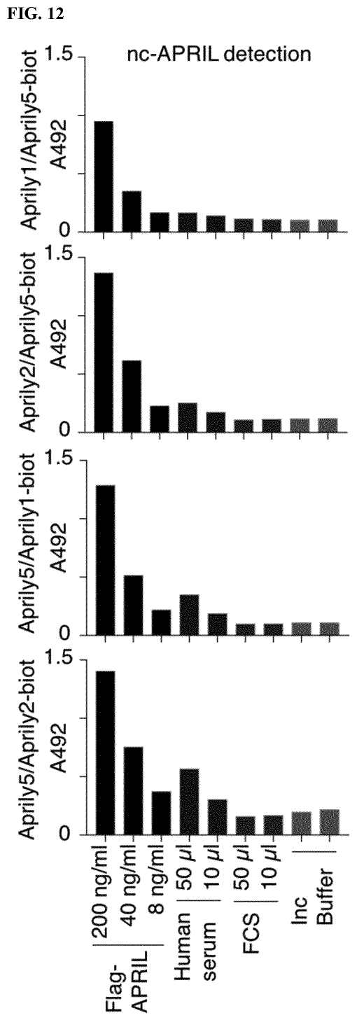

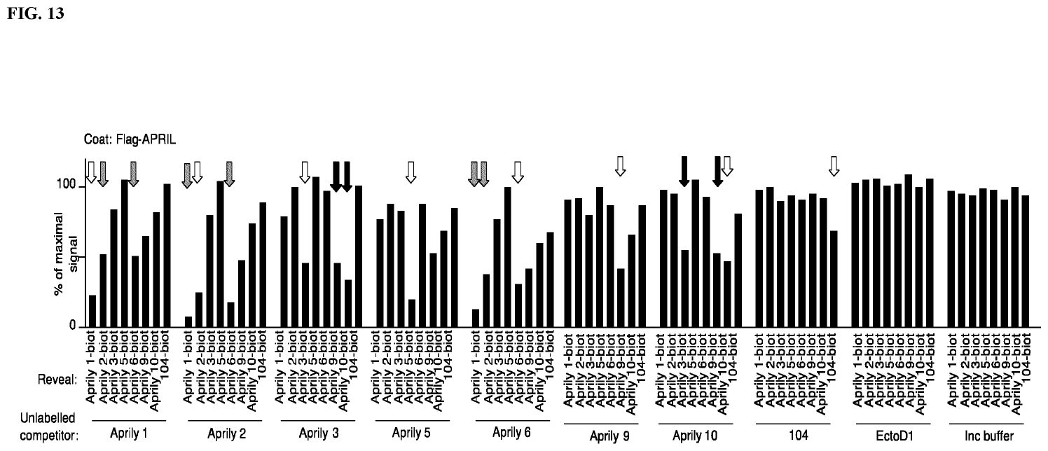

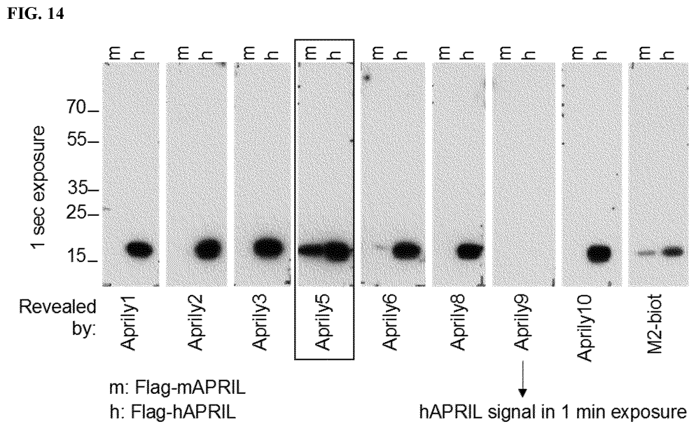

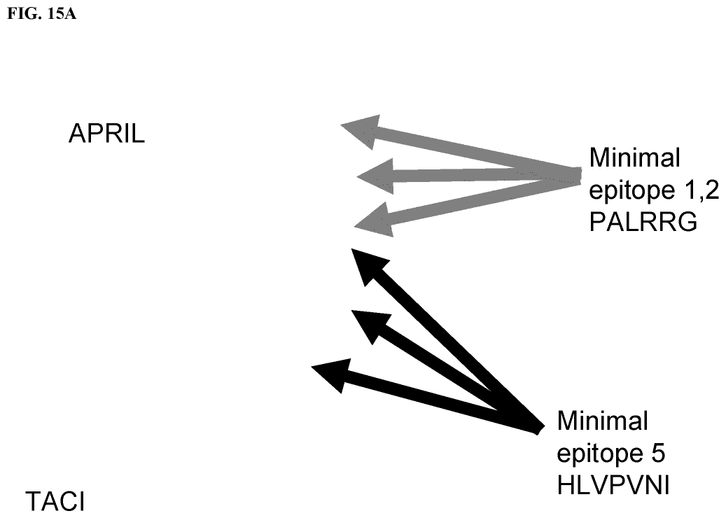

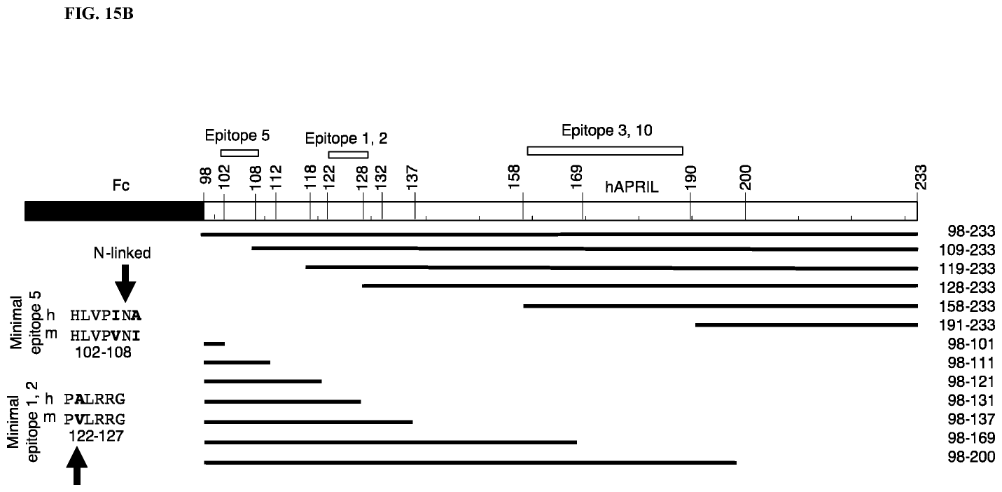

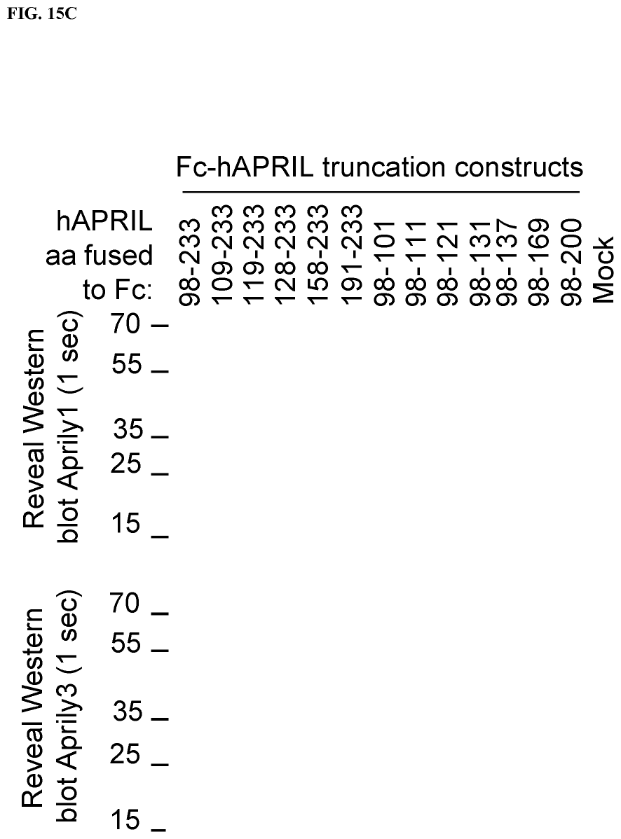

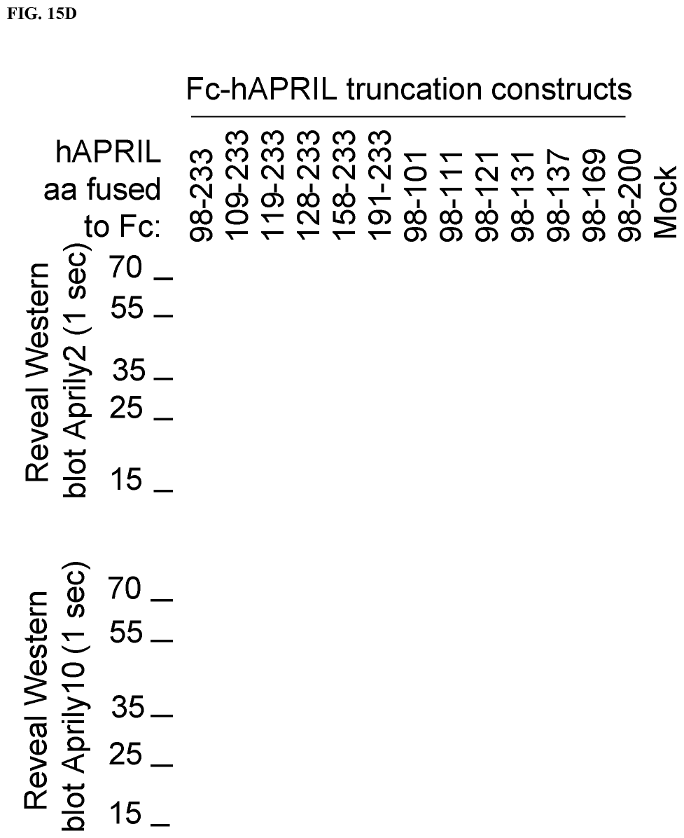

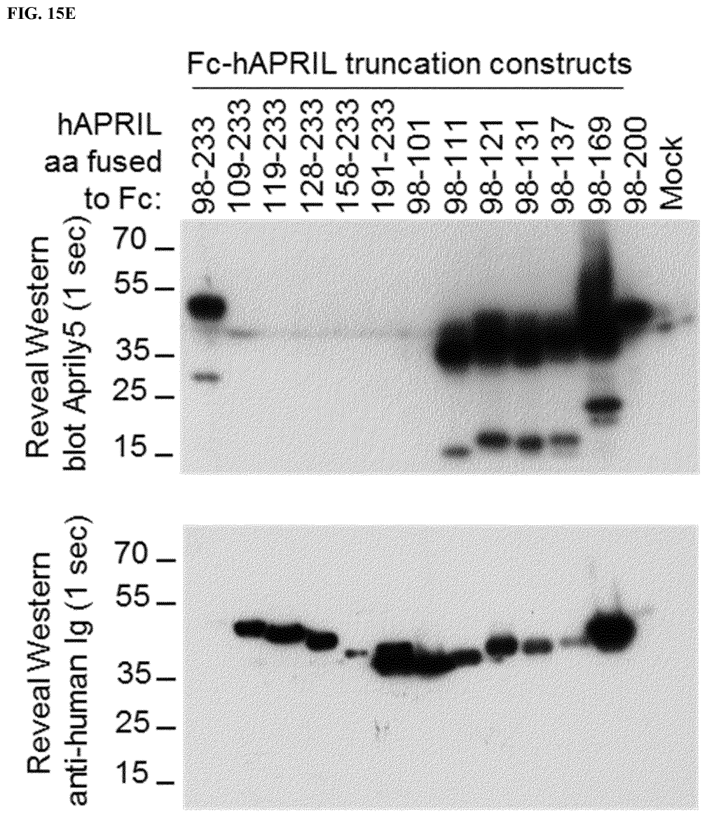

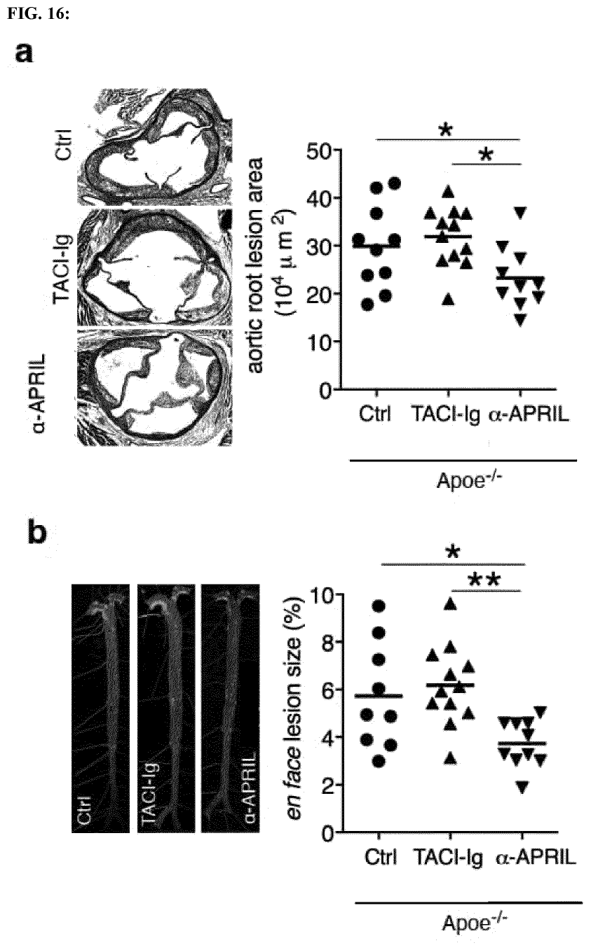

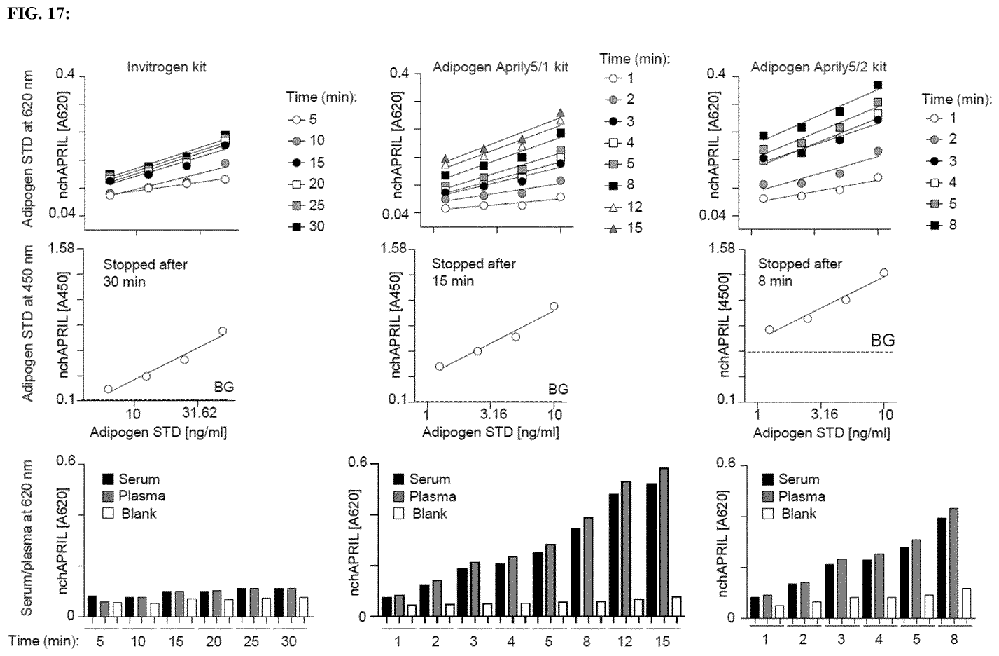

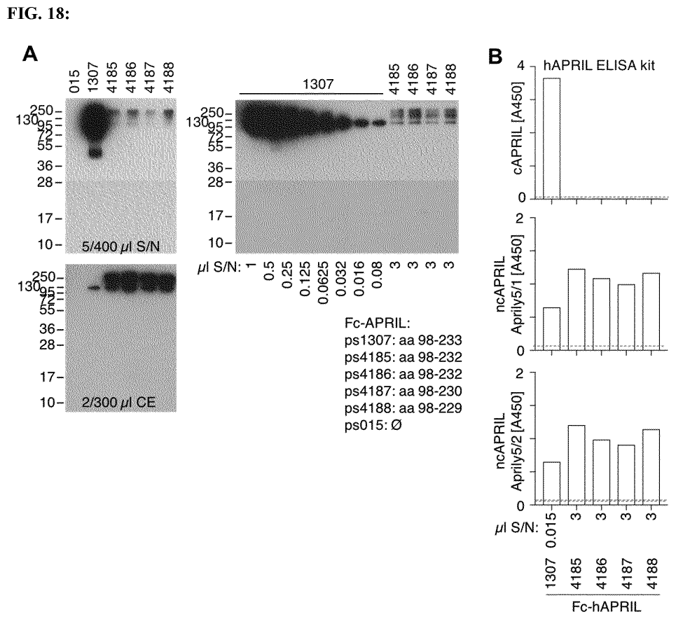

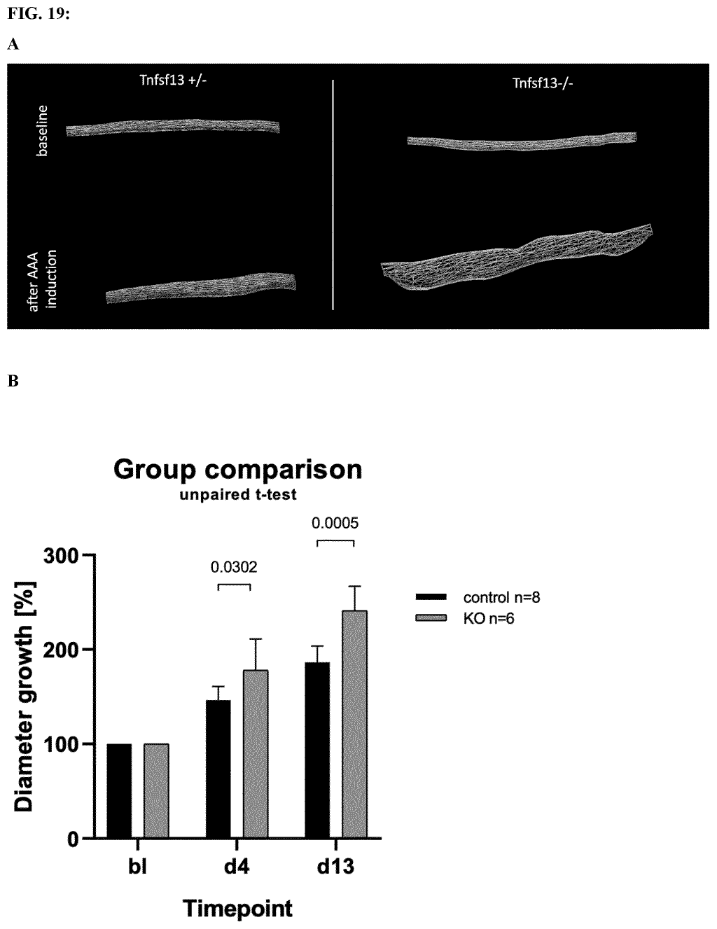

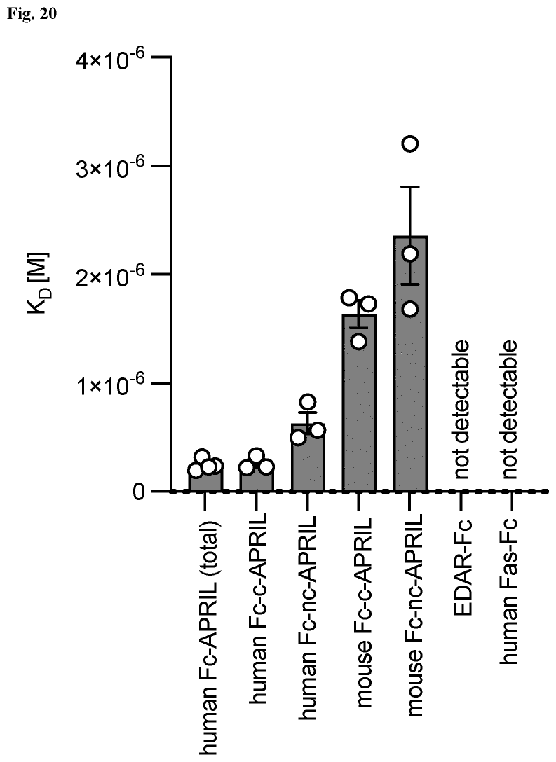

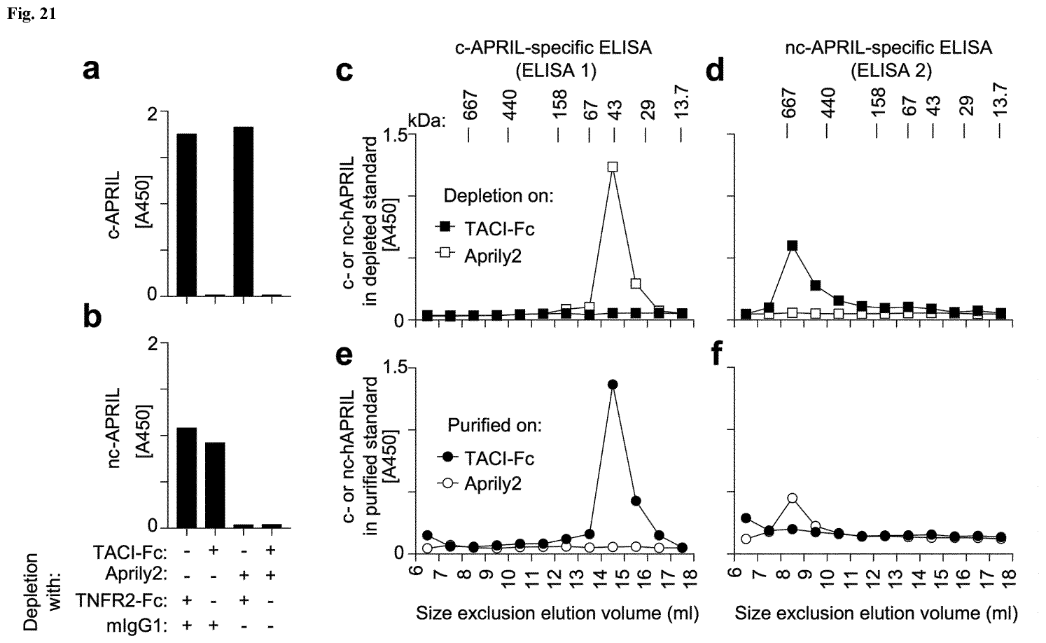

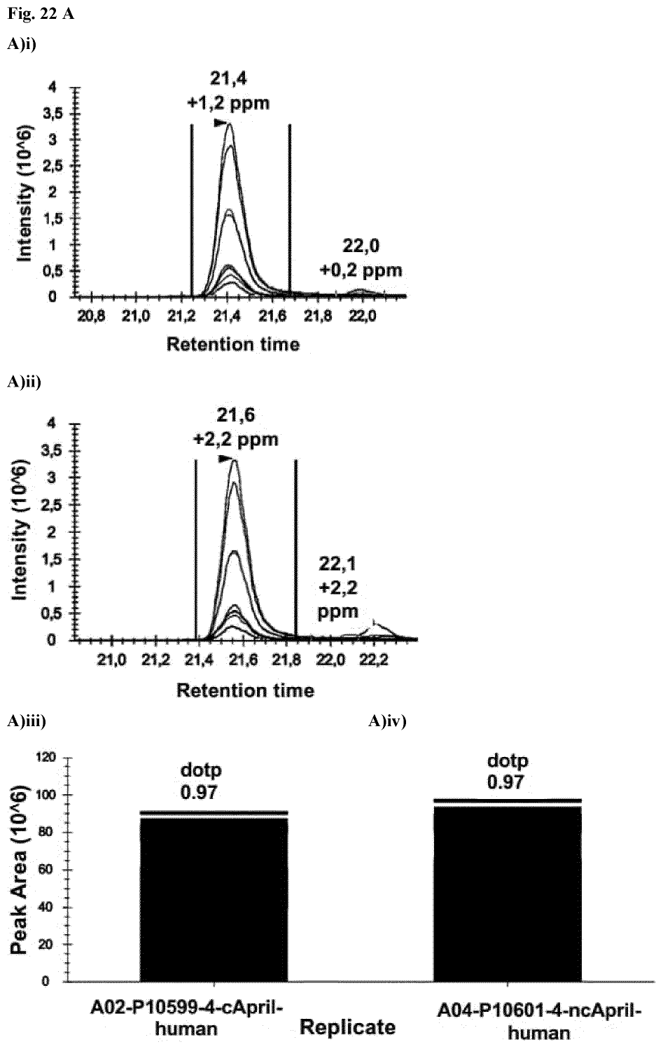

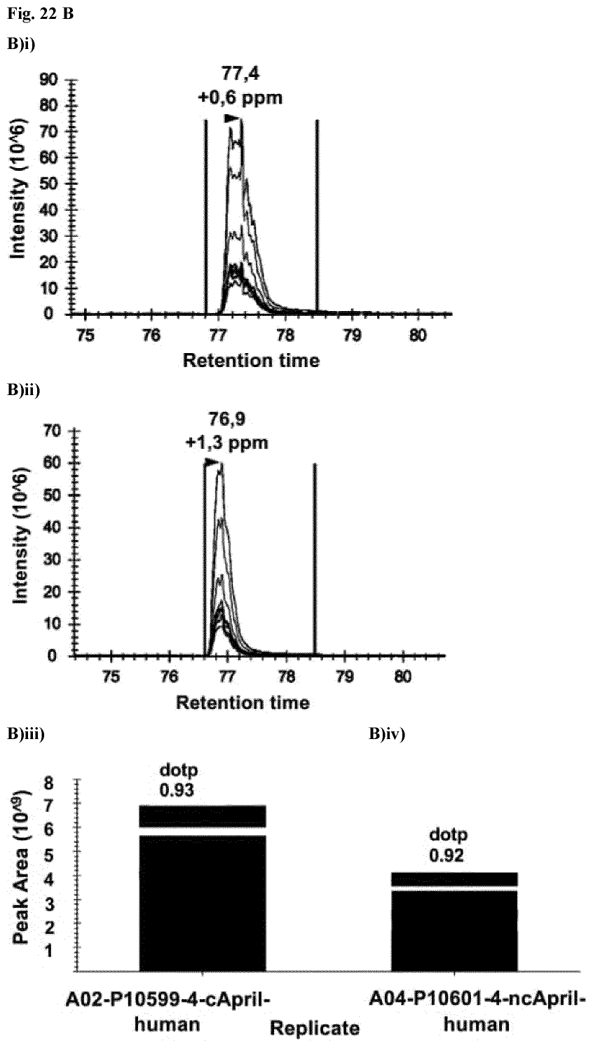

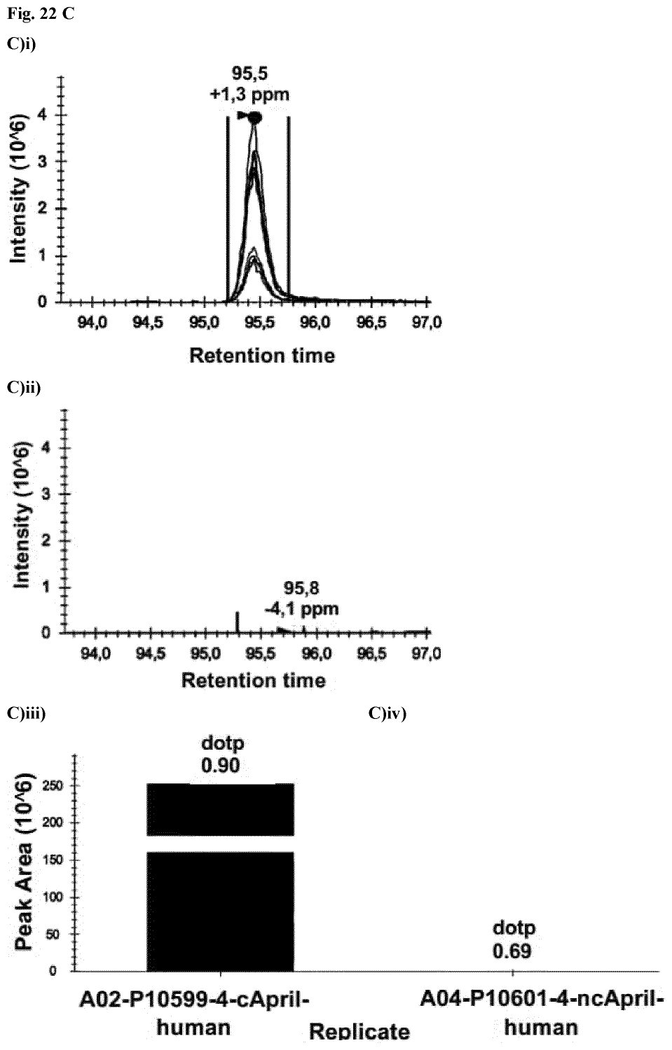

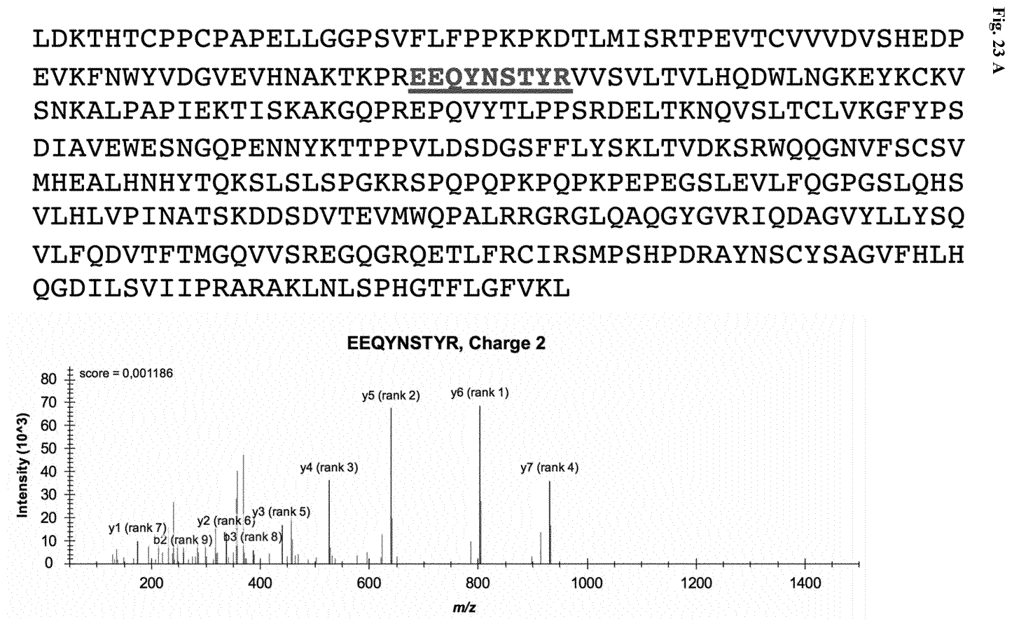

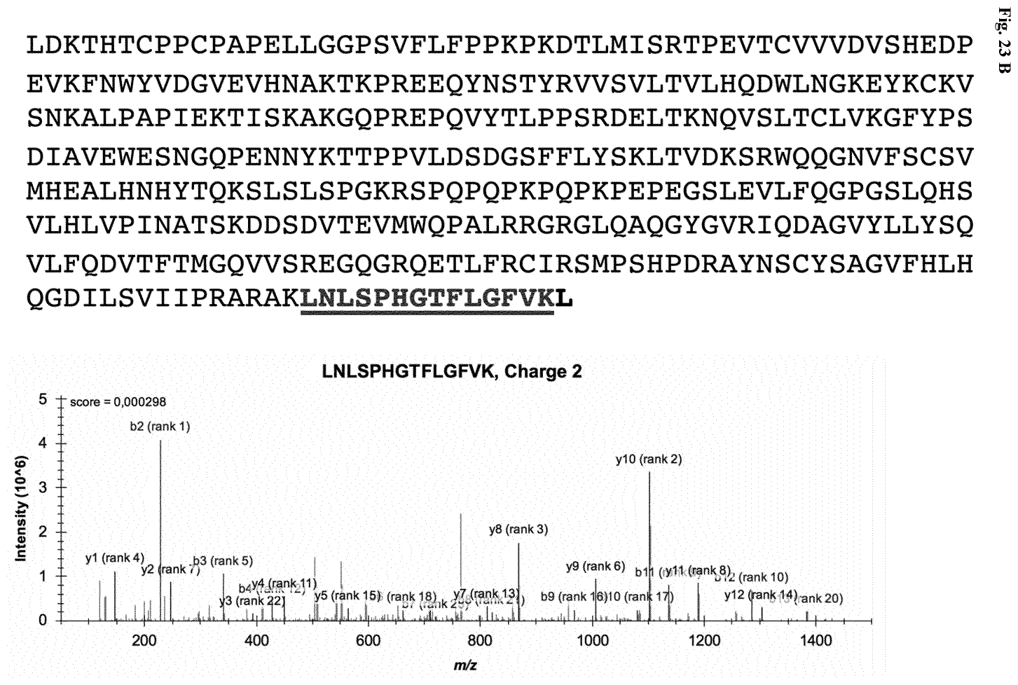

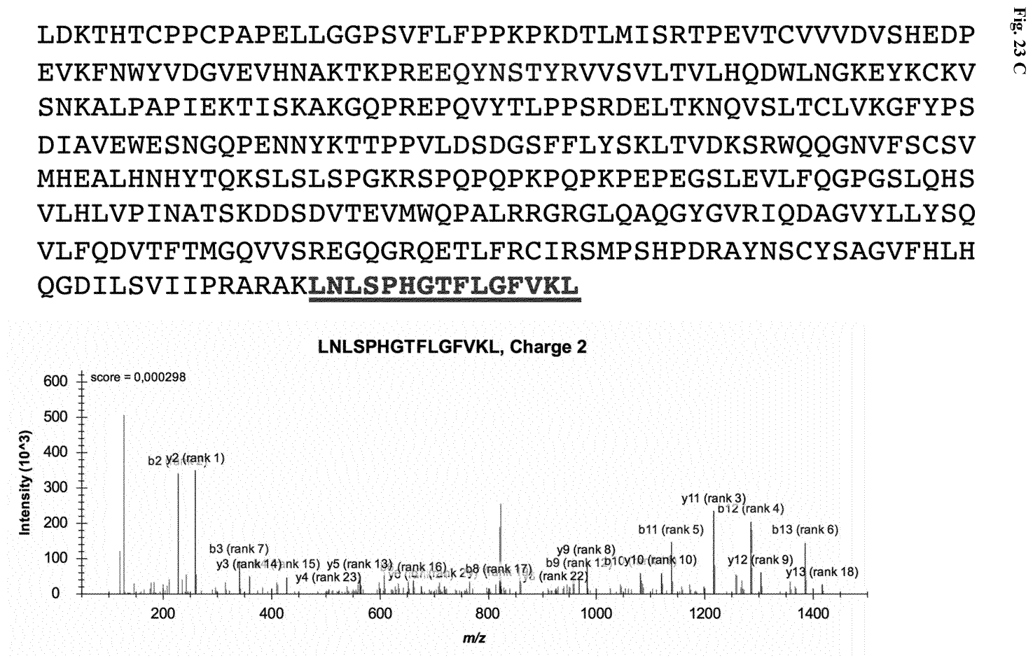

In addition, the inventors have recently identified a hitherto unknown form of APRIL that cannot bind to cognate immune receptors, which was termed non-canonical APRIL (nc-APRIL) (FIG. 2). Both the canonical form (c-APRIL, that can bind to immune receptors such as TACI) and nc-APRIL are present in recombinant Flag-APRIL produced from a cDNA and purified by affinity chromatography on anti-Flag, and also in normal human serum, and at low concentration in supernatants of the U937 human cell line. c-APRIL in recombinant Flag-APRIL or normal human serum is recognized by the Adipogen ELI SA and can be pre-depleted on immobilized TACI-Fc, but not on the irrelevant receptor TNFR2-Fc, whereas nc-APRIL is recognized by the Invitrogen ELISA (REF: BMS2008), and can be pre-depleted on the anti-APRIL monoclonal antibody Aprily 2, but not on the irrelevant isotype-matched antibody EctoD1. Depletion on Aprily 2 does not abolish signal in the Adipogen ELISA, and depletion on TACI-Fc does not abolish signal in the Invitrogen ELISA (REF: BMS2008), but depletion on both TACI-Fc and Aprily 2 abolishes signal in both ELISAs. Denatured Flag-APRIL behaved like nc-APRIL, suggesting that nc-APRIL exposes at least one epitope not normally accessible in c-APRIL. c-APRIL and nc-APRIL are coded by the same gene. Indeed, APRIL in conditioned supernatants of U937 cells contained both c-APRIL and nc-APRIL, but both forms became undetectable in several clones of APRIL-ko, or APRIL and BAFF double-ko U937 generated by CRISPR/Cas9 technology (FIG. 2).

### Example 3

The inventors have also identified and extensively characterized two ELISA assays that allow the specific detection of each form and thus provide an essential tool to investigate the biological role of nc-APRIL. Notably, nc-APRIL is detected by the same anti-APRIL antibody clone (Aprily 2) that detects APRIL in human arteries (FIG. 1) raising the possibility that a primary function of nc-APRIL is to interact with PGs.

As mentioned above, heparan sulfate PGs provide the platform both for the binding of LPL and for arresting TG-rich lipoproteins, thereby promoting TG degradation. Our aim is to investigate the effect of the interaction of APRIL with PGs with respect to triglyceride metabolism in a mouse model of atherogenic dyslipidemia. To investigate this, we study the well-established mouse model of atherogenic dyslipidemia that lack the LDL receptor (Ldlr−/−). These mice under chow diet feeding have moderately increased circulating levels of VLDL and LDL compared to wild-type mice. But when Ldlr−/− mice are fed an atherogenic diet (that contains 0.2% cholesterol and 21% fat), they develop markedly increased total cholesterol (>1,300 mg/dL) and TG (>700 mg/dL) levels in plasma and over time they develop atherosclerotic plaques.

### Example 4

To study the effect of APRIL in modulating TG metabolism via its capacity to bind to PGs, we treated Ldlr−/− mice with an anti-APRIL antibody that targets selectively the BCMA/TACI-binding site of APRIL and simultaneously strongly increases the binding of APRIL to HSPGs (FIG. 3).

### Example 5

The inventors found that Ldlr−/− mice that were treated with an isotype or an anti-mouse APRIL antibody (Apry 1.1; 5 mg/kg; administered biweekly) and fed an atherogenic diet for 8 weeks (which causes severe hypertriglyceridemia) had a 30 to 50% decrease in triglyceride levels in fasting plasma, in both female and male mice (FIG. 4). Moreover, there was a trend for reduced total plasma cholesterol levels in anti-APRIL antibody-treated mice, although this did not reach statistical significance (isotype: 1108±421 mg/dL vs anti-APRIL Ab: 866±206 mg/dL; n=10 mice per group). Notably, there was no difference in plasma triglyceride levels in Ldlr−/− April+/+ and Ldlr−/−April−/− littermate mice fed an atherogenic diet for 10 weeks (Ldlr−/− April+/+: 665±54 mg/dL vs Ldlr−/−April−/−: 597±43 mg/dL). These data suggest that the endogenous APRIL may not be sufficient to affect triglyceride metabolism (likely due to high occupancy by the immune receptors) and interventions that affect the capacity of APRIL to interact with HSPGs can be beneficial against hypertriglyceridemia in atherogenic dyslipidemia.

### Example 6

In line with reducing TG levels, anti-APRIL antibody treatment (Apry 1.1; see Example 5) also conferred an atheroprotective effect by reducing macrophage content in early atherosclerotic lesions (FIG. 5).

### Example 7

Notably, the effect of anti-APRIL antibody in lowering TG in plasma was not mediated via altering TG production by the liver nor via impacting body weight (FIG. 6), suggesting that an enhanced interaction between APRIL and PGs alters the mechanisms involved in enzymatic clearance of triglycerides.

### Example 8

Similar to LPL, APRIL binding to HSPGs can be completely competed by heparin. Thus, it is possible that APRIL competes with LPL for binding to PGs e.g. on the surface of the endothelium, and that antibody-mediated multimerization of endogenous APRIL promotes the release of LPL into the circulation, thereby promoting a more efficient hydrolysis of circulating triglyceride-rich lipoproteins (FIG. 7).

### Example 9

To obtain more insights into how antibody-mediated targeting of APRIL results in TG lowering, we will test in vivo additional monoclonal anti-mouse APRIL antibodies that have been generated and extensively validated in our laboratory. Like Apry 1.1, antibody 108 targets the TACI-binding site of APRIL, while others enhance the activity of APRIL through immune receptors (e.g. 104). But all of them, like Apry 1.1 (FIG. 3), strengthen the binding of APRIL to PGs (FIG. 8).

### Example 10

The inventors have developed a home-made ELISA assay to be able to detect nc-APRIL (FIG. 10) by using a combination of the anti-human APRIL Abs; Aprily 5, Aprily 2 and Aprily 1 as described in the validation data shown in FIG. 10.

### Example 11

In one embodiment of the invention, the ELISA assay according to the invention involves the following steps:
1. Coat the ELISA plate with 100 μl of Aprily 5 at 5 μg/ml. Incubate over-night at Room Temperature.
2. Block the plate using 100 μl of blocking buffer (PBS Tween 4% milk). Incubate for 1 hour at 37° C.
3. Wash 3 times with 300 μl of PBS Tween for each well 4 dry well.
4. Add 10 μl of serum samples plus 90 μl of incubation buffer (1:10 of blocking buffer). Incubate for 1 hour at 37° C.
5. Wash 3 times with 300 μl of PBS Tween for each well 4 dry well.
6. Add 100 μl Aprily 1-biot or Aprily 2-biot at 5 μg/ml in incubation buffer. Incubate for 1 hour at 37° C.
7. Wash 3 times with 300 μl of PBS Tween for each well 4 dry well.
8. Add 100 μl of streptavidin-HRP (1:4000 in incubation buffer). Incubate for 1 hour at 37° C.
9. Wash 3 times with 300 μl of PBS Tween for each well 4 dry well.
10. Reveal with 100 μl OPD. Wait for color development.
11. Stop with 50 μl of HCl 1N. Read at 492 nm in an ELISA reader.

### Example 12

Embodiments of the invention were compared to a commercially available ELISA Kit:

The hAPRIL Invitrogen (REF: BMS2008), Aprily 5/Aprily 1 and Aprily 5/Aprily 2 ELISA kits sensitivity was determined by the following method:
1. Check the levels of recombinant APRIL in hAPRIL ELISA kit from Adipogen (Cat: AG-45B-0012-KI01) in hAPRIL Invitrogen ELISA kit (REF: BMS2008) starting at 50 ng/ml in two fold dilutions or in Aprily 5/Aprily 1 or Aprily 5/Aprily 2 ELISA kits starting from 10 ng/ml in 2 fold dilutions or the levels of endogenous APRIL in 50 μl of normal human serum or plasma from PS (#1911-22) in these kits.
2. Add 3,3′,5,5′-tetramethylbenzidine (TMB).
3. Read the absorption at different time points at 620 nm.
4. Stop and read the absorption at 450 nm.

### Example 13

To examine a potential protective function of APRIL in human atherosclerotic CVD, we quantified serum nc-APRIL levels (using the Invitrogen ELISA REF: BMS2008) in 785 individuals with neurologically asymptomatic carotid atherosclerosis that were enrolled in the prospective clinical ICARAS study (Inflammation and Carotid Artery-Risk for Atherosclerosis Study). Kaplan-Meier analyses demonstrated a significant increase in cardiovascular and all-cause mortality with decreasing serum APRIL levels. The cumulative 12-year survival rates for cardiovascular mortality were 35.2%, 55.4% and 59.8% in the first, second and third tertile (log-rank P<0.0001; FIG. 9a). In addition, the cumulative 12-year survival rates for all-cause mortality were 55.3%, 75.0% and 75.5% in the first, the second and third tertile (log-rank P<0.0001; FIG. 9b). After adjustment for well-established cardiovascular risk factors including LDL-cholesterol and high-sensitive CRP in the cox proportional hazard model, patients within the first tertile displayed a significantly increased risk of all-cause mortality (adjusted HR 1.95, 95% CI 1.48 to 2.56, p<0.01; Table 1) and cardiovascular mortality (adjusted HR 2.20, 95% CI 1.56 to 3.12, p<0.01; Table 1) compared with patients within the third tertile.

We also quantified the serum canonical APRIL levels (using the Adipogen ELISA; described in FIG. 2) and we found that there is no correlation between cardiovascular or all-cause mortality with APRIL levels (Table 2).

TABLE 1
Results of univariate and multivariate Cox Regression analyses for nc-APRIL serum
levels of ICARAS study. 1ₛₜ Tertile includes patients with APRIL levels lower than
4.22 ng/ml, 2_{nd} tertile patients with April levels between 4.23 to 6.47 ng/ml, the
3_{rd} tertile patients with APRIL levels above 6.47 ng/ml. Adjusted for age, sex,
body mass index, smoking, hypertension, low-density lipoprotein cholesterol
levels, triglyceride levels, statin treatment, glycohemoglobin A1 level, diabetes
mellitus, history of myocardial infarction, history of peripheral artery disease,
history of stroke, and serum creatinine, intercellular adhesion molecule-1,
vascular cell adhesion molecule-1, and high-sensitivity C-reactive protein.
The third tertile serves as the reference category.

| | Univartiate | | | Multivariate | | |
|---|---|---|---|---|---|---|
| Variable | Hazard ratio | CI | P-value | Hazard ratio | CI | P-value |

All-Cause Mortality

| | | | | | | |
|---|---|---|---|---|---|---|
| 3_{rd} Tertile* | — | — | — | — | — | — |
| (>6.47 ng/ml) | | | | | | |
| 2_{nd} Tertile | 1.17 | 0.90-1.52 | 0.25 | 1.31 | 0.97-1.77 | 0.07 |
| (4.23 to | | | | | | |
| 6.47 ng/ml) | | | | | | |
| 1ₛₜ Tertile | 1.99 | 1.56-2.54 | <0.01 | 1.95 | 1.48-2.56 | <0.01 |
| (<4.22 ng/ml) | | | | | | |

Cardiovascular Mortality

| | | | | | | |
|---|---|---|---|---|---|---|
| 3_{rd} Tertile* | — | — | — | — | — | — |
| (>6.47 ng/ml) | | | | | | |
| 2_{nd} Tertile | 1.08 | 0.76-1.52 | 0.68 | 1.28 | 0.86-1.91 | 0.22 |
| (4.23 to | | | | | | |
| 6.47 ng/ml) | | | | | | |
| 1ₛₜ Tertile | 2.27 | 1.67-3.07 | <0.01 | 2.20 | 1.56-3.12 | <0.01 |
| (<4.22 ng/ml) | | | | | | |

*Reference Category
CI = confidence interval; HR = hazard ratio.

TABLE 2
Results of univariate and multivariate Cox Regression analyses for
APRIL serum levels in ICARAS study. 1st Tertile includes patients
with APRIL levels lower than 1.67 ng/ml, 2nd tertile patients with
April levels between 1.67 to 2.54 ng/ml, the 3rd tertile patients
with APRIL levels above 2.54 ng/ml. Adjusted for age, sex, body mass
index, smoking, hypertension, low-density lipoprotein cholesterol
levels, triglyceride levels, statin treatment, glycohemoglobin A1
level, diabetes mellitus, history of myocardial infarction, history
of peripheral artery disease, history of stroke, and serum creatinine,
intercellular adhesion molecule-1, vascular cell adhesion molecule-1,
and high-sensitivity C-reactive protein. The third tertile serves
as the reference category. CI = confidence interval; HR = hazard ratio.

| | Univariate | | | |
|---|---|---|---|---|
| | Hazard | | Hazard | |
| Variable | ratio | P-value | ratio | P-value |

All-Cause Mortality

| | | | | |
|---|---|---|---|---|
| 3rd Tertile* (>2.54 ng/dl) | — | — | — | — |
| | | | | — |
| | | | | — |
| 2nd Tertile (1.67 to 2.54 ng/dl) | 1.03 | 0.82 | 0.94 | 0.67 |
| 1st Tertile (<1.67 ng/dl) | 1.07 | 0.63 | 1.13 | 0.41 |

Cardiovascular Mortality

| | | | | |
|---|---|---|---|---|
| 3rd Tertile* (>2.54 ng/dl) | — | — | — | — |
| 2nd Tertile (1.67 to 2.54 ng/dl) | 0.89 | 0.38 | 0.78 | 0.21 |
| 1st Tertile (<1.67 ng/dl) | 1.0 | 0.89 | 1.19 | 0.35 |

*Reference Category Adjusted for age, sex, body mass index, smoking, hypertension, low-density lipoprotein cholesterol levels, triglyceride levels, statin treatment, glycohemoglobin A1 level, diabetes mellitus, history of myocardial infarction, history of peripheral artery disease, history of stroke, and serum creatinine, intercellular adhesion molecule-1, vascular cell adhesion molecule-1, and high-sensitivity C-reactive protein.

### Example 14

Abdominal aortic aneurysm (AAA) is a vascular disease that is often associated with advanced atherosclerosis and manifests with enlargement of the aorta due to artery wall weakening that leads progressively to artery rupture. AAA is an important cause of death worldwide. Considering the protective role of APRIL in atherosclerosis and its presence (in large amounts) in the arteries, we investigate the role of APRIL in a mouse model of AAA.

Methods: Briefly, topical, peri-adventitial application of elastase to the infrarenal abdominal aorta was performed in male C57BL/6 mice. Mice were anesthetized with 1.8-2% isoflurane. After median laparotomy, the intestines are gently exteriorized and soaked with saline to prevent necrosis. After dissecting the top layer of fat of the infrarenal aorta, the fat and connective tissue is used to create a unilateral periaortic pouch. Porcine pancreatic elastase is then applied into this pouch and left for 5 minutes before absorbing the elastase and flushing the abdomen with saline three times. Ultrasound was employed to monitor changes in anatomic parameters such as the maximum aortic diameter and vessel volume. In addition, ECG-gated kilohertz visualization (EKV) images will be taken for measurement of the maximum aortic diameter at maximal blood flow. The Vevo software (VEVO Lab 3.1.1) on the Vevo 3100 Imaging System (Visualsonics®) will be used for high-resolution measurements. The baseline ultrasound was performed one day before aneurysm surgery and then on day 4 and 13.

Results: We found that APRIL deficient mice develop larger aortic diameter upon AAA induction (see FIG. 19) and thus are more susceptible to AAA. These data indicate that APRIL plays a protective role in AAA and therapeutic strategies (like the ones described herein) that promote the accumulation of APRIL in the arteries can be considered for the treatment of AAA.

### Example 15

Mass Spectrometry Data Analysis (FIG. 22, 23)

HUVECs

Raw files were searched against a human database (containing 42,265 entries, downloaded from SwissProt (https://www.uniprot.org/) on 30 Dec. 2016) using Mascot version 2.3.02 (Matrix Science, London, UK) and Phenyx (GeneBio, Geneva, Switzerland) as search engines. Common contaminating proteins, such as porcine trypsin, were appended to the database. Mass tolerances were set to 4 ppm and 0.025 Da for precursor and fragment ions, respectively. Cleavage specificity was set to tryptic, however, one missed cleavage was allowed. Carbamidomethylation of cysteines was set as a static modification and oxidation of methionines was considered as a dynamic modification. A target-decoy search strategy was used to ensure an FDR of 1% on the protein level.

Human APRIL

Acquired raw data files were processed using Proteome Discoverer 2.4.1.15 SP1 for DDA experimental data or Skyline version 20.1.0.155 for PRM experimental data. A database search within PD 2.4 was done using the Sequest HT algorithm and Percolator validation software node (V3.04) to remove false positives with strict filtering at an FDR of 1% on PSM, peptide and protein levels. Searches were performed with full tryptic digestion against the human SwissProt database V2017.06 including a common contamination list with up to two miscleavage sites. Oxidation (+15.9949 Da) of methionine was set as variable modification, while carbamidomethylation (+57.0214 Da) of cysteine residues was set as fixed modification. Data were searched with mass tolerances of ±10 ppm and 0.025 Da on the precursor and fragment ions, respectively. Results were filtered to include peptide spectrum matches (PSMs) with Sequest HT cross-correlation factor (Xcorr) scores of >1 and high peptide confidence. For relative quantitative comparison of 26 selected APRIL tryptic peptide sequences, Skyline analysis was performed for canonical and non-canonical Fc-APRIL. The PD result file was used to build up a reference spectral library for Skyline analysis. Product ion chromatograms were extracted using the following Skyline settings: spectrum library ion match tolerance of 0.1 m/z; method match tolerance of 0.025 m/z; MS/MS filtering using targeted acquisition method at resolving power of 15,000 at m/z of 200. High-selectivity extraction was used for all matching scans. Integrated peak abundance values for selected peptides were exported.

### Example 16: Multivariate Cox Regression Analyses for Cardiovascular Mortality in the LURIC Study (nc-APRIL)

Adjusted for age (years), sex (male/female), C-reactive protein (mg dl⁻¹), triglycerides (mg dl⁻¹), total cholesterol levels (mg dl⁻¹), history of myocardial infarction (binary), history of stroke (binary), peripheral arterial disease (binary), body mass index (kg/m²), hypertension (binary), diabetes mellitus (binary), serum creatinine (mg dl⁻¹), haemoglobin 1AC (percent) (n=1,514).

TABLE 3
Overview of the LURIC study
LURIC (nc-APRIL)

| | Hazard | | | |
|---|---|---|---|---|
| Parameter | ratio | Lower .95 | Upper .95 | P values |
| Log nc-APRIL | 1.14 | 1.02 | 1.27 | 0.022 |
| age | 1.06 | 1.04 | 1.07 | <0.001 |
| sex | 0.70 | 0.53 | 0.92 | 0.011 |
| CRP | 1.38 | 0.95 | 2.01 | 0.087 |
| Triglycerides (log) | 0.84 | 0.63 | 1.12 | 0.230 |
| Total cholesterol (log) | 1.26 | 0.86 | 1.85 | 0.240 |
| Myocardial infarction | 1.06 | 0.82 | 1.37 | 0.666 |
| (no vs one) | | | | |
| Myocardial infarction | 1.93 | 1.33 | 2.81 | 0.001 |
| (no vs >one) | | | | |
| Stroke | 1.61 | 1.17 | 2.22 | 0.003 |
| Periph. Vasc. Disease | 1.83 | 1.33 | 2.50 | <0.001 |
| BMI | 0.98 | 0.95 | 1.02 | 0.331 |
| Isolated systolic | 0.83 | 0.65 | 1.06 | 0.140 |
| hypertension (>=140/<90) | | | | |
| Type II diabetes | 1.25 | 0.89 | 1.76 | 0.189 |
| Creatinine (log) | 1.86 | 1.19 | 2.90 | 0.007 |
| Hba1c (%) | 1.25 | 1.13 | 1.39 | <0.001 |

### Example 17: FAST-MI (French Registry of Acute ST-Elevation or Non-ST-Elevation Myocardial Infarction Clinical Study)

Multivariate Cox regression analyses for the FAST MI study. Circulating levels of nc-APRIL in patients at admission for acute myocardial infarction are associated with cardiovascular outcomes at follow-up. The probability of death during 2 years of follow-up as a function of baseline circulating plasma nc-APRIL levels (n=974). Results are expressed as hazard ratios (HR) with 95% CI.

TABLE 4
overview of the FAST MI study
FAST-MI (nc-APRIL)

| | Multivariate | | |
|---|---|---|---|
| Variable | Hazard ratio | CI | P-value |

All-cause death

| | | | |
|---|---|---|---|
| 3^{rd} Tertile | 1.91 | 1.25-2.91 | 0.0006 |
| 2^{nd} Tertile | 0.77 | 0.45-1.33 | ns |
| 1^{st} Tertile * | — | — | — |

* Reference Category

1. An antibody, or an antigen-binding fragment thereof, specifically binding to APRIL for use in the prevention and/or treatment of hypertriglyceridemia, metabolic syndrome, non-alcoholic steatohepatitis, diabetes mellitus type 2, atherogenic dyslipidemia, abdominal aortic aneurysm, cardiovascular events and/or atherosclerosis.

2. The antibody, or antigen-binding fragment thereof, according to claim 1, for use in the treatment and or prevention of hypertriglyceridemia.

3. The antibody, or antigen-binding fragment thereof, for use of claim 2, wherein the hypertriglyceridemia is at least one selected from the group of hypertriglyceridemia in metabolic syndrome, hypertriglyceridemia in non-alcoholic steatohepatitis-related, hypertriglyceridemia in diabetes mellitus type 2, hypertriglyceridemia in atherogenic, cardiovascular events with a history of hypertriglyceridemia and atherosclerosis with hypertriglyceridemia.

4. The antibody, or antigen-binding fragment thereof, according to claim 1, for use in the treatment and or prevention of abdominal aortic aneurysm.

5. The antibody, or antigen-binding fragment thereof, for use according to claim 1, wherein cardiovascular events comprise myocardial infarction, stroke, peripheral artery disease, angina pectoris and/or urgent hospitalization for angina leading to revascularization.

6. The antibody, or antigen-binding fragment thereof, for use according to claim 1 to 5, wherein the binding of the antibody or antigen-binding fragment thereof to APRIL results in an increased interaction of APRIL with proteoglycans.

7. The antibody, or antigen-binding fragment thereof, for use according to claim 6, wherein the proteoglycans are arterial and vascular proteoglycans.

8. The antibody, or antigen-binding fragment thereof, for use according to claim 6 or 7, wherein the proteoglycans are comprised in an extracellular matrix.

9. The antibody, or antigen-binding fragment thereof, according to any one of claims 1 to 8, wherein the binding of the antibody or antigen-binding fragment thereof to APRIL modulates the interaction of APRIL with at least one of its endogenous receptors.

10. The antibody, or antigen-binding fragment thereof, according to claim 9, wherein the binding of the antibody or antigen-binding fragment thereof to APRIL blocks the interaction of APRIL with the receptors TACI and BCMA.

11. An antibody, or antigen-binding fragment thereof or the antibody, or antigen-binding fragment thereof, for use according to any one of claims 1 to 10, wherein the antibody or antigen-binding fragment thereof
(a) comprises a variable heavy (VH) chain comprising CDR3 as defined in SEQ ID NO:10 and a variable light (VL) chain comprising CDR3 as defined in SEQ ID NO:13;
(b) comprises a variable heavy (VH) chain comprising CDR3 as defined in SEQ ID NO:17 and a variable light (VL) chain comprising CDR3 as defined in SEQ ID NO:20;
(c) comprises a variable heavy (VH) chain comprising CDR3 as defined in SEQ ID NO:24 and a variable light (VL) chain comprising CDR3 as defined in SEQ ID NO:27;
(d) comprises a variable heavy (VH) chain comprising CDR3 as defined in SEQ ID NO:31 and a variable light (VL) chain comprising CDR3 as defined in SEQ ID NO:34; or
(e) comprises a variable heavy (VH) chain comprising CDR3 as defined in SEQ ID NO:38 and a variable light (VL) chain comprising CDR3 as defined in SEQ ID NO:41.

12. The antibody, or antigen-binding fragment thereof, according to claim 11 or for use according to any one of claims 1 to 11, wherein the antibody or antigen-binding fragment thereof
(a) comprises a variable heavy (VH) chain comprising CDR1 as defined in SEQ ID NO:8, CDR2 as defined in SEQ ID NO:9 and CDR3 as defined in SEQ ID NO:10 and a variable light (VL) chain comprising CDR1 as defined in SEQ ID NO:12, CDR2 as defined in the sequence: YAS and CDR3 as defined in SEQ ID NO:13;
(b) comprises a variable heavy (VH) chain comprising CDR1 as defined in SEQ ID NO:15, CDR2 as defined in SEQ ID NO:16 and CDR3 as defined in SEQ ID NO:17 and a variable light (VL) chain comprising CDR1 as defined in SEQ ID NO:19, CDR2 as defined by the amino acid sequence: AAS and CDR3 as defined in SEQ ID NO:20;
(c) comprises a variable heavy (VH) chain comprising CDR1 as defined in SEQ ID NO:22, CDR2 as defined in SEQ ID NO:23 and CDR3 as defined in SEQ ID NO:24 and a variable light (VL) chain comprising CDR1 as defined in SEQ ID NO:26, CDR2 as defined by the amino acid sequence: GTN and CDR3 as defined in SEQ ID NO:27;
(d) comprises a variable heavy (VH) chain comprising CDR1 as defined in SEQ ID NO:29, CDR2 as defined in SEQ ID NO:30 and CDR3 as defined in SEQ ID NO:31 and a variable light (VL) chain comprising CDR1 as defined in SEQ ID NO:33, CDR2 as defined by the amino acid sequence: GTS and CDR3 as defined in SEQ ID NO:34; or
e) comprises a variable heavy (VH) chain comprising CDR1 as defined in SEQ ID NO:36, CDR2 as defined in SEQ ID NO:37 and CDR3 as defined in SEQ ID NO:38 and a variable light (VL) chain comprising CDR1 as defined in SEQ ID NO:40, CDR2 as defined by the amino acid sequence: LVS and CDR3 as defined in SEQ ID NO:41.

13. The antibody, or antigen-binding fragment thereof, according to claim 11 or 12 or for use according to any one of claims 1 to 12, wherein the antibody or antigen-binding fragment thereof
(a) comprises a variable heavy (VH) chain sequence comprising the amino acid sequence of SEQ ID NO:7 or a sequence having 90%, preferably 95% sequence identity to SEQ ID NO: 7; and
a variable light (VL) chain sequence comprising the amino acid sequence of SEQ ID NO:11 or a sequence having 90%, preferably 95% sequence identity to SEQ ID NO:11;
(b) comprises a variable heavy (VH) chain sequence comprising the amino acid sequence of SEQ ID NO:14 or a sequence having 90%, preferably 95% sequence identity to SEQ ID NO:14; and
a variable light (VL) chain sequence comprising the amino acid sequence of SEQ ID NO:18 or a sequence having 90%, preferably 95% sequence identity to SEQ ID NO:18;
(c) comprises a variable heavy (VH) chain sequence comprising the amino acid sequence of SEQ ID NO:21 or a sequence having 90%, preferably 95% sequence identity to SEQ ID NO:21; and
a variable light (VL) chain sequence comprising the amino acid sequence of SEQ ID NO:25 or a sequence having 90%, preferably 95% sequence identity to SEQ ID NO:25;
(d) comprises a variable heavy (VH) chain sequence comprising the amino acid sequence of SEQ ID NO:28 or a sequence having 90%, preferably 95% sequence identity to SEQ ID NO:28; and
a variable light (VL) chain sequence comprising the amino acid sequence of SEQ ID NO:32 or a sequence having 90%, preferably 95% sequence identity to SEQ ID NO:32; or
(e) comprises a variable heavy (VH) chain sequence comprising the amino acid sequence of SEQ ID NO:35 or a sequence having 90%, preferably 95% sequence identity to SEQ ID NO:35; and
a variable light (VL) chain sequence comprising the amino acid sequence of SEQ ID NO:39 or a sequence having 90%, preferably 95% sequence identity to SEQ ID NO:39.

14. The antibody, or antigen-binding fragment thereof, according to any one of claims 11 to 13 or for use according to any one of claims 1 to 13, wherein the antibody is an IgM, IgG1, IgG2a or IgG2b, IgG3, IgG4, IgA or IgE antibody.

15. The antibody, or antigen-binding fragment thereof, according to any one of claims 11 to 14 or for use according to any one of claims 1 to 14, wherein the antigen-binding fragment is a Fab fragment, an F(ab′) fragment, an Fv fragment or an scFv fragment.

16. A polynucleotide that encodes the antibody, or an antigen-binding fragment thereof, according to any one of claims 11 to 15.

17. The polynucleotide according to claim 16, wherein the polynucleotide encodes the antibody, or an antigen-binding fragment thereof, according to claim 13.

18. A pharmaceutical composition comprising the antibody, or antigen-binding fragment thereof, according to any one of claims 11 to 15 and a pharmaceutically acceptable carrier.

19. The pharmaceutical composition according to claim 18, comprising a further therapeutic agent.

20. The pharmaceutical composition according to claim 19, wherein the further therapeutic agent is selected from a group consisting of: fibrates, statins, agents that inhibit the expression of Angiopoietin-like protein 3 (ANGPTL3) or Apolipoprotein C-III (apoC-III), and agents that prevent the binding of auto-antibodies to GPIHBP1.

21. A method for quantifying the concentration of non-canonical APRIL (nc-APRIL) in a sample, the method comprising the steps of:
a) contacting the sample comprising nc-APRIL with a first monoclonal antibody specifically binding to a first epitope of nc-APRIL, wherein said first monoclonal antibody is an immobilized antibody;
b) contacting the mixture of step (a) with a second monoclonal antibody, wherein said second monoclonal antibody specifically binds to a second epitope of nc-APRIL;
c) detecting the binding of the second monoclonal antibody to immobilized nc-APRIL; and
d) quantifying the concentration of nc-APRIL in the sample according to the detected binding in step (c).

22. A method for quantifying the total concentration of c-APRIL and nc-APRIL in a sample, the method comprising the steps of:
a) contacting a denatured sample comprising nc-APRIL and/or c-APRIL with a first monoclonal antibody specifically binding to a first epitope of denatured nc-APRIL and c-APRIL, wherein said first monoclonal antibody is an immobilized antibody;
b) contacting the mixture of step (a) with a second monoclonal antibody, wherein said second monoclonal antibody specifically binds to a second epitope of denatured nc-APRIL and c-APRIL;
c) detecting the binding of the second monoclonal antibody to the immobilized forms of nc-APRIL and/or c-APRIL; and
d) quantifying the total concentration of nc-APRIL and c-APRIL in the sample according to the detected binding in step (c).

23. A method for quantifying the amount of c-APRIL in a sample, the method comprising the steps of:
a) quantifying the amount of nc-APRIL in a first portion of the sample with the method according to claim 21;
b) quantifying the total amount of nc-APRIL and c-APRIL in a second portion of the sample with the method according to claim 22, wherein the second portion of the sample has been denatured; and
c) quantifying the concentration of c-APRIL in the sample, wherein quantifying the concentration of c-APRIL in the sample involves subtracting the concentration of nc-APRIL in the sample from the total concentration of nc-APRIL and c-APRIL in the sample.

24. The method according to any one of claims 21 to 23, wherein the method is a sandwich enzyme-linked immunosorbent assay (ELISA) method.

25. The method according to any one of claims 21 to 24, wherein the second monoclonal antibody is conjugated to a detection moiety or a binding moiety.

26. The method according to claim 25, wherein the binding moiety is biotin.

27. The method according to any one of claims 21 to 26, wherein the first monoclonal antibody comprises a variable heavy (VH) chain sequence comprising the amino acid sequence of SEQ ID NO:56 or a sequence having 90%, preferably 95% sequence identity to SEQ ID NO:56; and a variable light (VL) chain sequence comprising the amino acid sequence of SEQ ID NO:60 or a sequence having 90%, preferably 95% sequence identity to SEQ ID NO:60; and wherein the second monoclonal antibody comprises
(a) a variable heavy (VH) chain sequence comprising the amino acid sequence of SEQ ID NO:42, or a sequence having 90%, preferably 95% sequence identity to SEQ ID NO:42; and a variable light (VL) chain sequence comprising the amino acid sequence of SEQ ID NO:46, or a sequence having 90%, preferably 95% sequence identity to SEQ ID NO:46; or
(b) a variable heavy (VH) chain sequence comprising the amino acid sequence of SEQ ID NO:49, or a sequence having 90%, preferably 95% sequence identity to SEQ ID NO:49; and a variable light (VL) chain sequence comprising the amino acid sequence of SEQ ID NO:53, or a sequence having 90%, preferably 95% sequence identity to SEQ ID NO:53.

28. The method according to any one of claims 21 to 26, wherein the first monoclonal antibody comprises
(a) a variable heavy (VH) chain sequence comprising the amino acid sequence of SEQ ID NO:42, or a sequence having 90%, preferably 95% sequence identity to SEQ ID NO:42; and a variable light (VL) chain sequence comprising the amino acid sequence of SEQ ID NO:46, or a sequence having 90%, preferably 95% sequence identity to SEQ ID NO:46; or
(b) a variable heavy (VH) chain sequence comprising the amino acid sequence of SEQ ID NO:49, or a sequence having 90%, preferably 95% sequence identity to SEQ ID NO:49; and a variable light (VL) chain sequence comprising the amino acid sequence of SEQ ID NO:53, or a sequence having 90%, preferably 95% sequence identity to SEQ ID NO:53; and
wherein the second monoclonal antibody comprises a variable heavy (VH) chain sequence comprising the amino acid sequence of SEQ ID NO:56 or a sequence having 90%, preferably 95% sequence identity to SEQ ID NO:56; and a variable light (VL) chain sequence comprising the amino acid sequence of SEQ ID NO:60 or a sequence having 90%, preferably 95% sequence identity to SEQ ID NO:60

29. A kit for determining the level of nc-APRIL in a sample, the kit comprising a first and a second monoclonal antibody, wherein both monoclonal antibodies bind to different epitopes of nc-APRIL.

30. A nephelometric assay for quantifying the concentration of non-canonical APRIL (nc-APRIL) in a sample, the assay comprising the steps of:
a) contacting a sample comprising nc-APRIL with at least one antibody, or antibody coupled to microparticles or microbeads specifically binding to nc-APRIL;
b) transmitting light to the mixture of step (a);
c) measuring a change in light scattering intensity of the mixture in response to the irradiation in step (b); and
d) quantifying the concentration of nc-APRIL in the sample according to the measurement in step (c).

31. The method according to claim 30, wherein steps (a) to (c) are repeated with at least one dilution of the sample comprising nc-APRIL and/or the at least one antibody.

32. The method according to any one of claims 30 to 31, wherein the at least one antibody specifically binding to nc-APRIL comprises
(a) a variable heavy (VH) chain sequence comprising the amino acid sequence of SEQ ID NO:42, or a sequence having 90%, preferably 95% sequence identity to SEQ ID NO:42; and a variable light (VL) chain sequence comprising the amino acid sequence of SEQ ID NO:46, or a sequence having 90%, preferably 95% sequence identity to SEQ ID NO:46; or
(b) a variable heavy (VH) chain sequence comprising the amino acid sequence of SEQ ID NO:49, or a sequence having 90%, preferably 95% sequence identity to SEQ ID NO:49; and a variable light (VL) chain sequence comprising the amino acid sequence of SEQ ID NO:53, or a sequence having 90%, preferably 95% sequence identity to SEQ ID NO:53; or
(c) a variable heavy (VH) chain sequence comprising the amino acid sequence of SEQ ID NO:56 or a sequence having 90%, preferably 95% sequence identity to SEQ ID NO:56; and a variable light (VL) chain sequence comprising the amino acid sequence of SEQ ID NO:60 or a sequence having 90%, preferably 95% sequence identity to SEQ ID NO:60

33. A method for predicting and/or diagnosing hypertriglyceridemia, metabolic syndrome, non-alcoholic steatohepatitis, diabetes mellitus type 2, atherogenic dyslipidemia, cardiovascular events and/or atherosclerosis, the method comprising the steps of:
(a) determining the concentration of non-canonical APRIL (nc-APRIL) in a sample that has been obtained from said subject;
(b) comparing the concentration of nc-APRIL that has been determined in step (a) to a reference value; and
(c) predicting and/or diagnosing hypertriglyceridemia, metabolic syndrome, non-alcoholic steatohepatitis and diabetes mellitus type 2, atherogenic dyslipidemia, cardiovascular events and/or atherosclerosis in said subject based on the comparison made in step (b).

34. A method for predicting mortality risk in subjects suffering from hypertriglyceridemia, metabolic syndrome, non-alcoholic steatohepatitis, diabetes mellitus type 2, atherogenic dyslipidemia, cardiovascular events and/or atherosclerosis, the method comprising the steps of:
(a) determining the concentration of nc-APRIL in a sample that has been obtained from said subject;
(b) comparing the concentration of nc-APRIL that has been determined in step (a) to a reference value; and
(c) determining the mortality risk of said subject based on the comparison made in step (b).

35. A method for determining whether a subject is susceptible to the treatment of hypertriglyceridemia, metabolic syndrome, non-alcoholic steatohepatitis, diabetes mellitus type 2, atherogenic dyslipidemia, cardiovascular events and/or atherosclerosis, the method comprising the steps of:
(a) determining the concentration of nc-APRIL in two or more samples that have been obtained from said subject at an earlier and a later time point;
(b) determining that said subject is susceptible to the treatment of hypertriglyceridemia, metabolic syndrome, non-alcoholic steatohepatitis and diabetes mellitus type 2, atherogenic dyslipidemia, cardiovascular events and/or atherosclerosis, if the concentration of nc-APRIL is higher in a sample that has been obtained at a later time point compared to a sample that has been obtained at an earlier time point; or determining that said subject is not susceptible to the treatment of hypertriglyceridemia, metabolic syndrome, non-alcoholic steatohepatitis, diabetes mellitus type 2, atherogenic dyslipidemia, cardiovascular events and/or atherosclerosis, if the concentration of nc-APRIL is similar or lower in a sample that has been obtained at a later time point compared to a sample that has been obtained at an earlier time point.

36. The method according to claim 35, wherein the earlier time point is before the beginning of the treatment and the later time point is after the beginning of the treatment; or wherein the earlier and the later time points are after the beginning of the treatment.

37. The method according to claim 35 or 36, wherein the treatment comprises the use of the antibody, or antigen-binding fragment thereof, of claims 11 to 15 or the pharmaceutical composition of claims 18 to 20.

38. The method according to any one of claims 33 to 37, wherein the cardiovascular events comprise myocardial infarction, stroke, peripheral artery disease, angina pectoris and/or urgent hospitalization for angina leading to revascularization.

39. The method according to any one of claims 33 to 38, wherein the sample is or comprises human serum or human plasma.

40. The method according to any one of claims 32 to 39, wherein the concentration of nc-APRIL is determined with at least one antibody specifically binding to nc-APRIL.

41. The kit according to claim 29 or the method for quantifying according to one of claims 21 to 27, wherein the first monoclonal antibody specifically binds to an epitope within the amino acid sequence SEQ ID NO:96 and/or the second monoclonal antibody specifically binds to an epitope within the amino acid sequence SEQ ID NO:64.

42. The kit according to claim 29 or the method for quantifying according to one of claims 21 to 26 or 28, wherein the first monoclonal antibody specifically binds to an epitope within the amino acid sequence SEQ ID NO:64 and/or the second monoclonal antibody specifically binds to an epitope within the amino acid sequence SEQ ID NO:96.

43. The nephelometric assay according to one of claims 30 to 32, wherein at least one antibody specifically binding to nc-APRIL binds to an epitope within the amino acid sequence SEQ ID NO:64 and/or at least one antibody specifically binding to nc-APRIL binds to an epitope within the amino acid sequence SEQ ID NO:96.

44. The method for predicting and/or diagnosing according to claim 40, wherein at least one antibody specifically binding to nc-APRIL binds to an epitope within the amino acid sequence SEQ ID NO:64 and/or at least one antibody specifically binding to nc-APRIL binds to an epitope within the amino acid sequence SEQ ID NO:96.

45. The method according to any one of claims 33 to 40, wherein the concentration of nc-APRIL is determined with the method according to any one of claim 21 or 41 to 42, the nephelometric assay according to one of claims 30 to 32 or 43, or the kit according to claim 29.

46. An antibody, or an antigen-binding fragment thereof, specifically binding to APRIL, wherein the antibody, or the antigen-binding fragment thereof, binds to an epitope within the amino acid sequence SEQ ID NO:64 or SEQ ID NO:96.

47. The antibody, or antigen-binding fragment thereof, according to claim 46 wherein the antibody, or the antigen-binding fragment thereof, binds to an epitope within the amino acid sequence SEQ ID NO:96.

48. The antibody, or antigen-binding fragment thereof, according to claim 46 wherein the antibody, or the antigen-binding fragment thereof, binds to an epitope within the amino acid sequence SEQ ID NO:64.
